(19) United States
(12) Patent Application Publication
(10) Pub. No.: US 2004/0009613 A1
(43) Pub. Date: Jan. 15, 2004

(54) Methods of identifying modulators of bromodomains

(76) Inventors: Ming-Ming Zhou, Greenwich, CT (US); Aneel K. Aggarwal, Edgewater, NJ (US); Eric Verdin, San Francisco, CA (US); Melanie Ott, San Francisco, CA (US)

Correspondence Address: KLAUBER & JACKSON, 411 HACKENSACK AVENUE, HACKENSACK, NJ, 07601

(21) Appl. No.: 10/209,201

(22) Filed: Jul. 31, 2002

Related U.S. Application Data
(62) Division of application No. 09/784,553, filed on Feb. 16, 2001.

(51) Int. Cl. G06F 19/00; G01N 33/48; G01N 33/50; G01N 33/543

(52) U.S. Cl. 436/518; 702/19

(57) ABSTRACT

The present invention provides the structural determination of a bromodomain determined by NMR spectroscopy. The present invention also provides binding partners for the bromodomain. The present invention further provides the structural determination of the Tat-P/CAF binding complex determined by NMR spectroscopy. In addition, the present invention provides methodology for related drug discovery using high throughput drug screening or structure based rational drug design using the three-dimensional data.

### CROSS-REFERENCE TO RELATED APPLICATIONS

The present application is a divisional of U.S. Ser. No. 09/784,553 filed Feb. 16, 2001, which is a continuation-in-part application copending U.S. Ser. No. 09/510,314 filed Feb. 22, 2000, the disclosures of which are hereby specifically incorporated by reference in their entirety. Applicants claim the benefits of this application under 35 U.S.C. §120.

### FIELD OF THE INVENTION

The present invention provides the three-dimensional structure of a histone acetyltransferase bromodomain. The three-dimensional structural information is included in the invention. The present invention also identifies for the first time, that bromodomains can bind to binding partners that comprise an acetylated lysine. The interaction between bromodomains and their binding partners play a crucial role in various cellular functions, including in the regulation/modulation of DNA transcription. Therefore, the present invention provides procedures for identifying agents that can modulate the interaction of bromodomains and their binding partners by high throughput drug screening and/or through the use of rational drug design based on the three-dimensional data provided herein.

### BACKGROUND OF THE INVENTION

In recent years great strides have been made in the elucidation of the steps involved in intercellular and intracellular signaling. Indeed, the individual steps of the cascade of events involved in a number of cellular signal transduction processes have been determined. For example, intercellular signal transduction generally begins with an intercellular ligand binding the extracellular portion of a receptor of the plasma membrane. The bound receptor then either directly or indirectly initiates the activation of one or more cellular factors. An activated cellular factor may act as transcription factor by entering the nucleus to interact with its corresponding genomic response element, or alternatively, it may interact with other cellular factors depending on the complexity of the process. In either case, one or more transcription factors ultimately bind to one or more specific genomic response elements. This binding plays a crucial role in the up and/or down regulation of the transcription of the specific genes that are under the control of these genomic response elements. However, the process of re-organizing the chromatin of eukaryotic cells, which is a prerequisite for the binding of the transcription factor to the genomic response elements, has remained a mystery.

Chromatin contains several highly conserved histone proteins including: H3, H4, H2A, H2B, and H1. These histone proteins package eukaryotic DNA into repeating nucleosomal units that are folded into higher-order chromatin fibers [Luger and Richmond, Curr. Opin. Genet. Dev. 8:140-146 (1998)]. A portion of the histone that comprises roughly a quarter of the protein protrudes from the chromatin surface, and is thereby sensitive to proteolytic enzymes [van Holde, in Chromatin (Rich, A,. ed., Springer, New York) pages111-148 (1988); Hect et al., Cell 80:583-592 (1995)]. This portion of the histone is known as the “histone tail”. Histone tails tend to be free for protein-protein interaction, and are also the portion of the histone most prone to post-translational modification. Such post-translational modification includes acetylation, phosphorylation, methylation, ubiquitination, and ADP-ribosylation [van Holde, in Chromatin (Rich, A,. ed., Springer, New York) pages111-148 (1988)].

Of all classes of proteins, histones are amongst the most susceptible to post-translational modification. Perhaps the best studied post-translational modification of histones is the acetylation of specific lysine residues [Grunstin, M., Nature, 389:349-352 (1997)]. Indeed, acetylation of histone lysine residues has been suggested to play a pivotal role in chromatin remodeling and gene activation. Consistently, distinct classes of enzymes, namely histone acetyltransferases (HATs) and histone deacetylases (HDACs), acetylate or de-acetylate specific histone lysine residues [Struhl, Genes Dev. 12:599-606 (1998)].

Nearly all known nuclear HATs contain an approximately 110 amino acid sequence known as the bromodomain [Jeanmougin et al., Trends in Biochemical Sciences, 22:151-153 (1997)], a protein motif that was initially discovered in Drosophila brahma protein. Bromodomains are found in a large number of chromatin-associated proteins and have now been identified in approximately 70 human proteins, often adjacent to other protein motifs [Jeanmougin et al., Trends in Biochemical Sciences, 22:151-153 (1997); Tamkun et al., Cell, 68:561-572 (1992): Hanes et al., Nucleic Acids Research, 20:2603 (1992)]. Proteins that contain a bromodomain often contain a second bromodomain. However, despite the wide occurrence of bromodomains and their likely role in chromatin regulation, their three-dimensional structure and binding partners heretofore have remained unknown.

Therefore, there is a need to identify a binding partner for a bromodomain. In addition, there is a need to identify agonists or antagonists to the bromodomain-binding partner complex. Since a preferred method of drug-screening relies on structure based drug design, there is also a need to determine the three-dimensional structure of a bromodomain. In this case, once the three dimensional structure of bromodomain is determined, potential agonists and/or potential antagonists can be designed with the aid of computer modeling [Bugg et al., Scientific American, December:92-98 (1993); West et al., TIPS, 16:67-74 (1995); Dunbrack et al., Folding & Design, 2:27-42 (1997)]. However, heretofore the three-dimensional structure of the bromodomain has remained unknown. Therefore, there is a need for obtaining a form of the bromodomain that is amenable for NMR analysis and/or X-ray crystallographic analysis. Furthermore, there is a need for the determination of the three-dimensional structure of the bromodomain. Finally, there is a need for procedures for related structural based drug design predicated on such structural data.

### SUMMARY OF THE INVENTION

The present invention provides, for the first time, that bromodomains bind to acetyl-lysine residues of proteins. The present invention also provides the three-dimensional structure of a bromodomain as well as the three-dimensional structure of a bromodomain-acetyl-histamine complex. The structural information provided can be employed in methods of identifying drugs that can modulate the cellular processes that involve bromodomain-acetyl-lysine interactions. These interactions include chromatin remodeling, which is a required step in eukaryotic transcription. In a particular embodiment, the three-dimensional structural information is used in the identification and/design of an inhibitor of leukemia. In another embodiment, the three-dimensional structural information is used in the identification and/design of an inhibitor of HIV-1 infection and/or AIDS.

The present invention provides an isolated nucleic acid that encodes a peptide consisting of about 21 to 40 amino acids that comprises a ZA loop of a bromodomain. In a preferred embodiment the peptide comprises about 23 to 34 amino acids. The isolated nucleic acid can further comprise a heterologous nucleotide sequence. In a preferred embodiment the peptide comprises the amino acid sequence of SEQ ID NO:3. In another embodiment the peptide comprises the amino acid sequence of SEQ ID NO:43. In particular embodiments the ZA loop is obtained from the bromodomain having the amino acid sequence of SEQ ID NO:7, or SEQ ID NO:8, or SEQ ID NO:9, or SEQ ID NO:10, or SEQ ID NO:11, or SEQ ID NO:12, or SEQ ID NO:13, or SEQ ID NO:14, or SEQ ID NO:15, or SEQ ID NO:16, or SEQ ID NO:17, or SEQ ID NO:18, or SEQ ID NO:19, or SEQ ID NO:20, or SEQ ID NO:21,or SEQ ID NO:22, or SEQ ID NO:23, or SEQ ID NO:24, or SEQ ID NO:25, or SEQ ID NO:26, or SEQ ID NO:27, or SEQ ID NO:28, or SEQ ID NO:29, or SEQ ID NO:30, or SEQ ID NO: or SEQ ID NO:31, or SEQ ID NO:32, or SEQ ID NO:33, or SEQ ID NO:34, or SEQ ID NO:35, or SEQ ID NO:36, or SEQ ID NO:37, or SEQ ID NO:38, or SEQ ID NO: or SEQ ID NO:39, or SEQ ID NO:40, or SEQ ID NO:41, or SEQ ID NO:42.

The present invention further provides a recombinant DNA molecule that comprises an isolated nucleic acid of the present invention, as described above, with or without a heterologous nucleotide sequence. Such a recombinant DNA molecule can be operatively linked to an expression control sequence and can be part of an expression vector. The present invention further provides a cell that comprises such an expression vector. The cell can be either a eukaryotic or a prokaryotic cell. The present invention further provides a method of expressing the peptides of the present invention or fragments thereof in this cell. One such method comprises culturing the cell in an appropriate cell culture medium under conditions that provide for expression of the peptide by the cell.

The present invention further provides a peptide consisting of about 21 to 40 amino acids that comprises a ZA loop of a bromodomain. In a preferred embodiment the peptide comprises about 23 to 34 amino acids. The present invention also provides fusion proteins or peptides comprising these peptides. In a preferred embodiment the peptide comprises the amino acid sequence of SEQ ID NO:3. In another embodiment the peptide comprises the amino acid sequence of SEQ ID NO:43. In yet another preferred embodiment the peptide comprises the amino acid sequence of SEQ ID NO:48. In particular embodiments, the ZA loop is obtained from the bromodomain having the amino acid sequence of SEQ ID NO:7, or SEQ ID NO:8, or SEQ ID NO:9, or SEQ ID NO:10, or SEQ ID NO:11, or SEQ ID NO:12, or SEQ ID NO:13, or SEQ ID NO:14, or SEQ ID NO:15, or SEQ ID NO:16, or SEQ ID NO:17, or SEQ ID NO:18, or SEQ ID NO:19, or SEQ ID NO:20, or SEQ ID NO:21, or SEQ ID NO:22, or SEQ ID NO:23, or SEQ ID NO:24, or SEQ ID NO:25, or SEQ ID NO:26, or SEQ ID NO:27, or SEQ ID NO:28, or SEQ ID NO:29, or SEQ ID NO:30, or SEQ ID NO: or SEQ ID NO:31, or SEQ ID NO:32,or SEQ ID NO:33, or SEQ ID NO:34, or SEQ ID NO:35, or SEQ ID NO:36, or SEQ ID NO:37, or SEQ ID NO:38, or SEQ ID NO: or SEQ ID NO:39, or SEQ ID NO:40, or SEQ ID NO:41, or SEQ ID NO:42.

The present invention also provides antibodies raised against the peptides/proteins of the present invention, or raised against an antigenic fragment of these proteins/fragments. In a particular embodiment an antibody is raised against a fragment of the ZA loop of a bromodomain. In another embodiment an antibody is raised against a fragment of a protein or peptide that comprises an acetyl-lysine, wherein the protein or peptide can bind to a bromodomain. Such fragments can be conjugated to a carrier protein or be part of a fusion protein. In one embodiment the antibody is a polyclonal antibody. In another embodiment, the antibody is a monoclonal antibody. A hybridoma that makes the monoclonal antibody is also part of the present invention. In a particular embodiment the antibody is a chimeric antibody. Antibodies that can specifically recognize acetyl-lysine residues involved bromodomain binding are also part of the present invention.

In another aspect of the present invention a method is provided for identifying a compound that modulates the affinity of a bromodomain for a ligand (and/or protein) that comprises an acetylated lysine or an analog of an acetylated lysine (see FIG. 12). One such embodiment comprises contacting the bromodomain and the ligand in the presence of a compound under conditions that, the bromodomain and the ligand bind in the absence of the compound. The affinity of the bromodomain for the ligand is then determined (e.g., measured). A compound is identified as a compound that modulates the affinty of the bromodomain for the ligand when there is a change in the affinity of the bromodomain for the ligand in the presence of the compound. When the affinity of the bromodomain for the ligand increases in the presence of the compound, the compound is identified as a promoting agent for the bromodomain-ligand complex. When the affinity of the bromodomain for the ligand decreases in the presence of the compound, the compound is identified as an inhibitor of the bromodomain-ligand complex. In a preferred embodiment, the compound to be tested is pre-selected by performing rational drug design with the set of atomic coordinates obtained from one or more of Tables 1-6. More preferably the selecting is performed in conjunction with computer modeling. In a particular embodiment, the compound is selected by performing rational drug design with the set of atomic coordinates obtained from a set of atomic coordinates defining the three-dimensional structure of a bromodomain consisting of the amino acid sequence of SEQ ID NO:7 alone or with acetyl-histamine.

The present invention also provides a method of identifying a compound that modulates the stability of a bromodomain-ligand binding complex. Preferably the ligand comprises either an acetyl-lysine or an analog of acetyl-lysine. One such embodiment comprises contacting the bromodomain-ligand binding complex in the presence of the compound under conditions in which the bromodomain-ligand binding complex forms in the absence of the compound. The stability of the bromodomain-ligand binding complex is then determined (e.g., measured). A compound is identified as a compound that modulates the stability of the bromodomain-ligand binding complex when there is a change in the stability of the bromodomain-ligand binding complex in the presence of that compound. When the stability of the bromodomain-ligand binding complex increases in the presence of the compound, the compound is identified as a stabilizing agent. When the stability of the bromodomain-ligand binding complex decreases in the presence of the compound, the compound is identified as an inhibitor. In a preferred embodiment, the compound to be tested is pre-selected by performing rational drug design with the set of atomic coordinates obtained from one or more of Tables 1-6. More preferably the selecting is performed in conjunction with computer modeling. In a particular embodiment, the compound is selected by performing rational drug design with the set of atomic coordinates obtained from a set of atomic coordinates defining the three-dimensional structure of a bromodomain consisting of the amino acid sequence of SEQ ID NO:7 alone or with acetyl-histamine.

As one of skill in the art of drug development would readily understand, the potential drugs selected by the above methodologies can be refined by re-testing in appropriate drug assays, including those disclosed herein. Chemical analogs of such potential drugs can be obtained (either through chemical synthesis or drug libraries) and be analogously tested. Therefore, methods comprising successive iterations of the steps of the individual drug assays, as exemplified herein, using either repetitive or different binding studies, or transcription activation studies or other such studies are envisioned in the present invention. In addition, potential drugs may be identified first by rapid throughput drug screening, as described below, prior to performing computer modeling on a potential drug using the three-dimensional structure of the bromodomain.

The present invention further comprises the potential, selected, and putative compounds (drugs) identified by the methods of the present invention, as well as the final drugs themselves identified with the methods of the present invention.

The present invention further provides a method for identifying a potential binding partner for a protein (e.g., a histone) comprising an acetyl-lysine. One such embodiment comprises contacting the protein with a polypeptide comprising a bromodomain. In a preferred embodiment the bromodomain comprises the amino acid sequence of SEQ ID NO:3. In particular embodiments the bromodomain has the amino acid sequence of SEQ ID NO:7, or SEQ ID NO:8, or SEQ ID NO:9, or SEQ ID NO:10, or SEQ ID NO:11, or SEQ ID NO:12, or SEQ ID NO: 13, or SEQ ID NO:14, or SEQ ID NO:15, or SEQ ID NO:16, or SEQ ID NO:17, or SEQ ID NO: 18, or SEQ ID NO:19, or SEQ ID NO:20, or SEQ ID NO:21, or SEQ ID NO:22, or SEQ ID NO: 23, or SEQ ID NO:24, or SEQ ID NO:25, or SEQ ID NO:26, or SEQ ID NO:27, or SEQ ID NO: 28, or SEQ ID NO:29, or SEQ ID NO:30, or SEQ ID NO: or SEQ ID NO:31, or SEQ ID NO: 32, or SEQ ID NO:33, or SEQ ID NO:34, or SEQ ID NO:35, or SEQ ID NO:36, or SEQ ID NO: 37, or SEQ ID NO:38, or SEQ ID NO: or SEQ ID NO:39, or SEQ ID NO:40, or SEQ ID NO: 41, or SEQ ID NO:42.

The present invention further provides a method for identifying a protein having a bromodomain. One such embodiment comprises contacting a cellular extract with a peptide comprising an acetyl-lysine and/or an acetyl-lysine analog.

The present invention further provides agents that can inhibit the binding of a bromodomain with a protein comprising an acetyl-lysine. In one embodiment the agent is and 13). One particular analog of acetyl-lysine is acetyl-histamine. In still another embodiment the agent is an antibody that recognizes an acetyl-lysine of a protein binding partner of a bromodomain. In a preferred embodiment the agent is an antibody raised against a ZA loop of a bromodomain. These agents can be used as pharmaceuticals in compositions that contain a pharmaceutically acceptable carrier for example, or in the various drug assays of the present invention, serving as controls to demonstrate specificity.

The present invention further provides an apparatus that comprises a representation of a bromodomain or a bromodomain-ligand complex (e.g., the Tat-P/CAF complex). One such apparatus is a computer that comprises the representation of the bromodomain or a bromodomain-ligand complex in computer memory. In one embodiment, the computer comprises a machine-readable data storage medium which contains data storage material that is encoded with machine-readable data which comprises the atomic coordinates from a bromodomain or a bromodomain-ligand complex. Preferably the computer comprises a machine-readable data storage medium which contains data storage material that is encoded with machine-readable data which comprises a portion or all of the structural coordinates contained in Tables 1-6 and 10-14. In one embodiment, the computer comprises a machine-readable data storage medium which contains data storage material that is encoded with machine-readable data which comprises the structural coordinates for the Tat-P/CAF complex. More preferably the computer further comprises a working memory for storing instructions for processing the machine-readable data, a central processing unit coupled to both the working memory and to the machine-readable data storage medium for processing the machine readable data into a three-dimensional representation of the Tat-P/CAF complex, for example. In a preferred embodiment, the computer also comprises a display that is coupled to the central-processing unit for displaying the three-dimensional representation.

In addition, the present invention provides methods of identifying compounds that modulate the affinity of P/CAF for Tat that is acetylated at the lysine residue at position 50 of SEQ ID NO:45. In one such embodiment the method comprises contacting the bromodomain of P/CAF or a fragment thereof with a binding partner in the presence of the compound under conditions in which the bromodomain of P/CAF and the binding partner bind in the absence of the compound. The affinity of the bromodomain of P/CAF and the binding partner is then determined (e.g., measured). When there is a change in the affinity of the bromodomain of P/CAF for the binding partner in the presence of the compound, the compound is identified as a modulator. In one embodiment of this type the binding partner is Tat that is acetylated at the lysine residue at position 50 of SEQ ID NO:45. In a preferred embodiment the binding partner is a fragment of Tat comprising an acetyl-lysine at position 50. In still another embodiment the binding partner is an analog of the fragment of Tat comprising an acetyl-lysine at position 50. When the affinity of the bromodomain of P/CAF for the binding partner increases in the presence of the compound, the compound is identified as a Tat-P/CAF complex promoting agent, whereas when the affinity of the bromodomain of P/CAF for the binding partner decreases in the presence of the compound, the compound is identified as an inhibitor of the Tat-P/CAF complex.

In a preferred embodiment the compound is selected by performing rational drug design with the set of atomic coordinates obtained from one or more of Tables 1-5 and 10-14. More preferably the selection is performed in conjunction with computer modeling. Compounds selected by these methods are also part of the present invention. Preferably the compound is a small organic molecule. More preferably the compound is an analog of acetyl-lysine. Even more preferably, the compound is not included in FIG. 13.

The present invention also provides methods of identifying a compound that modulates the stability of the binding complex formed between P/CAF and Tat that is acetylated at the lysine residue at position 50 of SEQ ID NO:45. In one such embodiment the method comprises contacting the bromodomain of P/CAF or a fragment thereof with a binding partner in the presence of the compound under conditions in which the bromodomain of P/CAF and the binding partner bind in the absence of the compound. The stability of the bromodomain of P/CAF and the binding partner is then determined (e.g., measured). When there is a change in the stability of the binding complex between the bromodomain of P/CAF and the binding partner in the presence of the compound, the compound is identified as a modulator. In one embodiment of this type the binding partner is Tat that is acetylated at the lysine residue at position 50 of SEQ ID NO:45. In a preferred embodiment the binding partner is a fragment of Tat comprising an acetyl-lysine at position 50. In still another embodiment the binding partner is an analog of the fragment of Tat comprising an acetyl-lysine at position 50. When the stability of the bromodomain of P/CAF for the binding partner increases in the presence of the compound, the compound is identified as a stabilizing agent, whereas when the stability of the bromodomain of P/CAF for the binding partner decreases in the presence of the compound, the compound is identified as an inhibitor of the Tat-P/CAF complex. In a preferred embodiment the compound is selected by performing rational drug design with the set of atomic coordinates obtained from one or more of Tables 1-5 and 10-14. More preferably the selection is performed in conjunction with computer modeling. Compounds identified by these methods are also part of the present invention. Preferably the compound is an analog of acetyl-lysine. More preferably the compound is a small organic molecule not included in FIG. 13.

The present invention also provides agents that can modulate the binding of P/CAF and Tat. In a preferred embodiment the agent is a small organic molecule. Preferably the agent inhibits and/or destabilizes the binding of P/CAF with Tat. Preferably the agent is an analog of acetyl-lysine. More preferably the agent is not included in FIG. 13.

Another aspect of the present invention provides methods of preventing, and/or retarding the progression and/or treating HIV infection in an individual. One such method employs administering to the individual compounds that modulate the Tat-P/CAF complex selected by performing rational drug design with the set of atomic coordinates obtained from one or more of Tables 1-5 and 10-14. In a preferred embodiment the compound administered is an acetyl-lysine analog. In a particular embodiment this compound is a small organic molecule contained in FIG. 13. Preferably the compound either de-stabilizes or inhibits the Tat-P/CAF complex.

Accordingly, it is a principal object of the present invention to provide the three-dimensional coordinates of a bromodomain, and a bromodomain complexed with acetyl-histamine. It is a further object of the present invention to provide the three-dimensional coordinates of the Tat-P/CAF complex.

It is an object of the present invention to provide an assay for identifying proteins that contain bromodomains that bind proteins that comprise acetyl-lysine, that can modulate the bromodomain-acetyl-lysine binding complex, and that can inhibit the binding of a bromodomain to a protein containing acetyl-lysine. It is a further object of the present invention to provide methods of identifying drugs that can modulate the Tat-P/CAF binding complex, and that can inhibit the binding/formation of the Tat-P/CAF binding complex. It is a further object of the present invention to provide methods that incorporate the use of rational design for identifying such drugs. Further, it is an object of the invention to provide a method of identifying drugs that can treat leukemia and that can treat, retard the progression, prevent and/or cure AIDS.

These and other aspects of the present invention will be better appreciated by reference to the following drawings and Detailed Description.

### BRIEF DESCRIPTION OF THE DRAWINGS

FIG. 1. Structure-based sequence alignment of a selected number of bromodomains. The sequences were aligned based on the NMR-derived structure of the P/CAF bromodomain, and the predicated four α-helices are shown in green boxes. Bromodomains are grouped on the basis of the sequence and/or functional similarities as described by Jeanmougin et al., [Trends in Biochemical Sciences, 22:151-153 (1997)]. Residue numbers of the P/CAF bromodomain are indicated above its sequence. Three absolutely conserved residues, corresponding to Pro751, Pro767, and Asn803 in the P/CAF bromodomain, are shown in red. Highly conserved residues are colored in blue. The residues of the P/CAF bromodomain that interact with acetyl-histamine, as determined by intermolecular NOEs, are indicated by asterisks. The ZA loop, which is critical for acetyl-lysine binding, for each of the indicated bromodomains is also identified. The underlined residues were changed individually by site-directed mutagenesis to Ala. Genbank accession numbers for the proteins are as indicated in Table 8, in the Example below, along with the SEQ ID NOs. for the bromodomain sequences.

FIGS. 2A-2H depict the structure of the P/CAF bromodomain. FIGS. 2A-2B shows the stereoview of the C_{α} trace of 30 superimposed NMR-derived structures of the bromodomain (residues 722-830). The N-terminal four residues (SKEP) which are structurally disordered are omitted for clarity. For the final 30 structures, the root-mean-square deviations (RMSDs) of the backbone and all heavy atoms are 0.63±0.11 Å and 1.15±0.12 Å for residues 723-830, respectively. The RMSDs of the backbone and all heavy atoms for the four α-helices (residues 727-743, 770-776, 785-802, and 807-827), are 0.34±0.04 Å and 0.87±0.06 Å, respectively. FIGS. 2C-2D show the stereoview of the bromodomain structures from the bottom of the protein, which is rotated approximately 90° from the orientation in FIGS. 2A-2B. FIG. 2E shows the Ribbons [Carson, M., J. Appl. Crystallogr. 24:958-961 (1991)] depiction of the averaged minimized NMR structure of the P/CAF bromodomain. The orientation of FIG. 2E is as shown in FIGS. 2A-2B. FIGS. 2F-2G are schematic representations of the overall topology of the up-and-down four-helix bundle folds with the opposite handedness. The left-handed fold is seen in bromodomain, cytochrome b₅, and T4 lysozyme (left, FIG. 2F), whereas the right-handed four-helix bundles are observed in proteins such as hemerythrin and cytochrome b₅₆₂ (right, FIG. 2G) [Richardson, J., Adv.Protein Chem., 34:167-339 (1989); Presnell and Cohen, Proc. Natl. Acad. Sci. USA 86:6592-6596 (1989)]. FIG. 2H is a molecular surface representation of the electrostatic potential (blue=positive; red=negative) of the bromodomain calculated in GRASP [Nicholls et al., Biophys. J. 64:166-170 (1993)]. The hydrophobic and aromatic residues (Tyr809, Tyr802, Tyr760, Ala757, and Val752) located between the ZA and BC loops are indicated.

FIGS. 3A-3C show the binding of the P/CAF bromodomain to AcK. FIG. 3A shows the superimposed region of the 2D ¹⁵N—HSQC spectra of the bromodomain (approximately 0.5 mM) in its free form (red) and complexed to the AcK-containing H4 peptide (molar ratio 1:6) (black). FIG. 3B is the Ribbon and dotted-surface diagram of the bromodomain depicting the location of the lysine-acetylated H4 peptide binding site. The color coding reflects the chemical shift changes (Δδ) of the backbone amide ¹H and ¹⁵N resonances upon binding to the AcK peptide as observed in the ¹⁵N—HSQC spectra. The normalized weighted average of the chemical shift changes was calculated by Δₐᵥ/Δₘₐₓ=[Δδ²_{NH}+Δδ²_{N}/25)/2]^{1/2}/Δₘₐₓ, where Δₘₐₓ is the maximum weighted chemical shift difference observed for Tyr809 (0.16 ppm). The backbone atoms are color-coded in red, yellow, or green for residues that have Δₐᵥ/Δₘₐₓ of >0.6 (Tyr809, Glu808, Asn803, and Ala757), 0.2-0.6 (Ala813, Tyr802, Tyr760, and Val752), or <0.2 (Cys812, Ser807, Cys799, Phe796, and Phe748), respectively. The non-perturbed residues are shown in blue. FIG. 3C shows the chemical structures of acetyl-lysine, acetyl-histamine, and acetyl-histidine.

FIG. 4 depicts the acetyl-lysine binding pocket. This is the Ribbons [Carson, M., J. Appl. Crystallogr. 24:958-961 (1991)] depiction of a portion of the P/CAF bromodomain complexed with the acetyl-histamine. The ligand is color-coded by atom type.

FIGS. 5A-5B show the binding of various bromodomains from P/CAF, CBP and TIF1b to the N-terminal biotinylated and lysine-acetylated Tat peptide that was immobilized on streptavidin agarose.

FIGS. 6A-6D shows the lysine-acetylated HIV-1 Tat protein interactions with bromodomains using 2D 1H-15N—HSQC spectra of the P/CAF or CBP bromodomain in the presence (red) or absence (black) of the lysine-acetylated peptides. Binding of the P/CAF bromodomain to the Tat AcK 50 peptide SYGR-AcK-KRRQRC (SEQ ID NO:50) is shown in FIG. 6A, to the Tat AcK 28 peptide TNCYCK-AcK-CCFH (SEQ ID NO:58) is shown in FIG. 6B, and to histone H4 AcK16 peptide SGRGKGGKGLGKGGA-AcK-RHRK (SEQ ID NO:59) is shown in FIG. 6C. FIG. 6D shows the binding of the CBP of the bromodomain to the Tat AcK50 peptide. AcK is an acetyl-lysine residue

FIG. 7 is a bar graph of the measurement of superinduction of Tat transactivation activity by P/CAF. Tat-KK is the wild type Tat protein, and Tat-RR is the double mutant Tat carrying lysine to arginine mutations at K50 and K51 positions.

FIGS. 8A-8B show a western blot assay to detect P/CAF interaction with the Tat protein. Note that the protein-protein interaction was only observed with the wild type Tat but not with the Tat K50R/K51R mutant protein. The FLAG was joined to the Tat peptide, whereas the HA-tag was joined to P/CAF.

FIG. 9 depicts the structure of the P/CAF bromodomain in the complex with the lysine-acetylated Tat peptide (SYGR-AcK-KRRQRC, SEQ ID NO:50, where AcK is acetyl-lysine residue). The side chains of the amino acid residues on both the protein (green) and peptide (dark orange) that showed intermolecular NOEs in the NMR spectra are displayed.

FIGS. 10A-10B shows the results of the mutational analyses of the P/CAF bromodomain binding to the HIV-1 Tat. FIG. 10A shows the effects of the point mutation of the individual residues of the bromodomain to alanine on the protein binding to the lysine-acetylated Tat peptide. FIG. 10B is an assessment of the peptide residue mutation on its binding to the P/CAF bromodomain.

FIG. 11 depicts a schematic of a computer comprising a central processing unit (“CPU”), a working memory, a mass storage memory, a display terminal, and a keyboard that are interconnected by a conventional bidirectional system bus. The computer can be used to display and manipulate the structural data of the present invention.

FIG. 12 depicts the chemical structure common to the acetyl-lysine analogs of the present invention. R₁, R₂, and R₃ can be H, CH₃, a halogen (e.g., F, Cl, Br, I etc.), OH, SH, or NH₃⁺. R4 can be an alkyl (including a peptide/protein attached thereto such as a peptide comprising an acetyl-lysine in which the “N” of the structure depicted is the epsilon nitrogen (i.e., N^{ε}) of a lysyl residue), or an aryl group. See also FIG. 13 for examples.

FIG. 13 depicts examples of acetyl-lysine analogs.

### DETAILED DESCRIPTION OF THE INVENTION

The present invention identifies a general binding partner (ligand) for the protein motif known as the bromodomain. Indeed, by combining structural and site-directed mutagenesis studies the present invention demonstrates that bromodomains can interact specifically with acetyl-lysine (AcK), making them the first protein modules known to exhibit such interactions. Like other modular domains, such as Src homology-2 (SH2) and phosphotyrosine binding (PTB) domains, which specifically interact with phosphotyrosine-containing proteins, the bromodomain/acetyl-lysine recognition provides a means to regulate protein-protein interactions via protein lysine acetylation. The nature of the acetyl-lysine recognition by the bromodomain is similar to that of histone acetyltransferase interaction with acetyl-CoA. The present invention therefore couples for the first time, the functionality of the bromodomain with the HAT activity of coactivators in the regulation of gene transcription. The present invention further provides both a nuclear magnetic resonance (NMR) structure of the bromodomain from the HAT coactivator P/CAF (p300/CBP-associated factor) as well as the structure for the P/CAF bromodomain in complex with acetyl-histamine. The structure reveals an unusual left-handed up-and-down four-helix bundle.

The results disclosed herein explain prior deletion experiments which showed that the bromodomain is indispensable for the function of GCN5 in yeast. Bromodomain-AcK binding also appears to be important for the assembly and activity of multiprotein complexes in transcriptional activation. The results reported herein therefore form the foundation for identifying specific biological ligands and for defining the molecular mechanisms by which the extensive family of bromodomains participate in chromatin remodeling and transcriptional activation.

As disclosed herein, the binding partner for the bromodomain is a peptide or protein comprising an acetyl-lysine (AcK). Interestingly, whereas a free acetyl-lysine does not appear to bind the bromodomain, an analog of the acetyl-lysine, acetyl-histamine, does. This is most likely due to the additional charge present in the free amino acid. Consistently, free acetyl-histidine also does not to bind the bromodomain.

In addition, as disclosed herein, the gene transactivation of HIV-1 Tat protein requires lysine-acetylation at amino acid residue 50 of Tat (see SEQ ID NO:45) by the transcription co-activator p300/CBP and the subsequent formation of a binding complex between the Tat having the acetylated lysine with P/CAF. The binding complex between P/CAF and Tat is mediated via the bromodomain of P/CAF and the acetylated Tysine of Tat. Indeed, this binding is required for the gene transactivation activity of Tat and thus, for HIV-1 expression and replication.

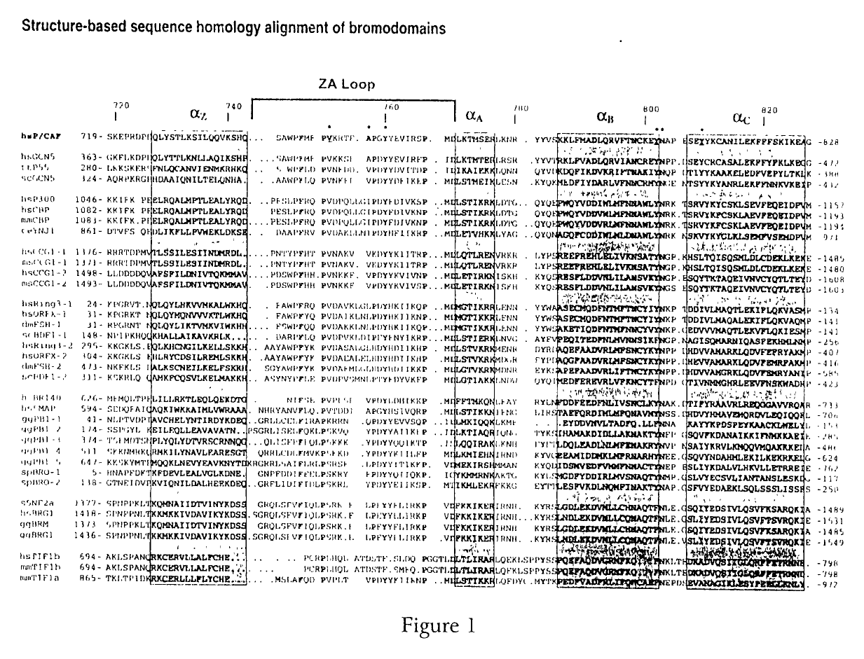

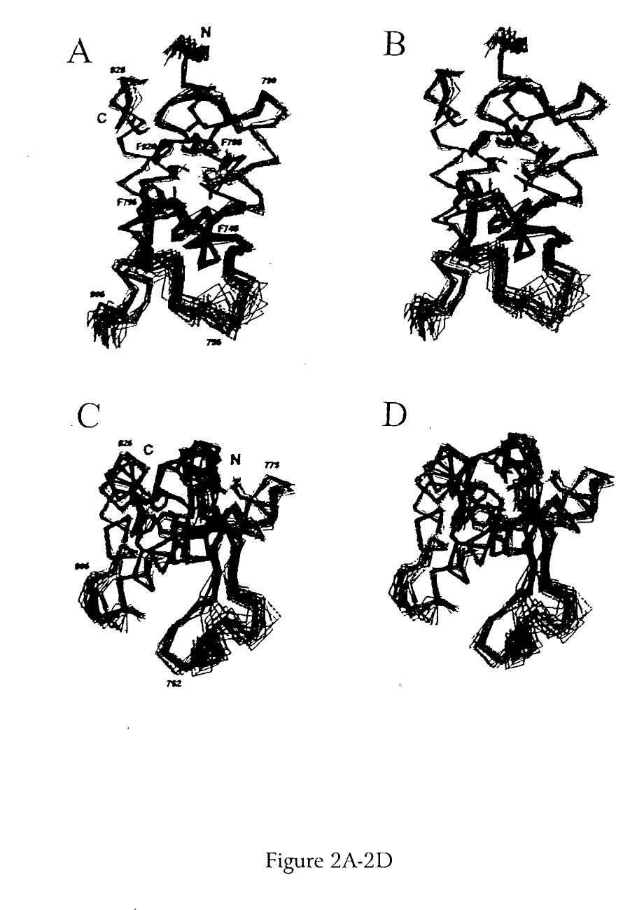

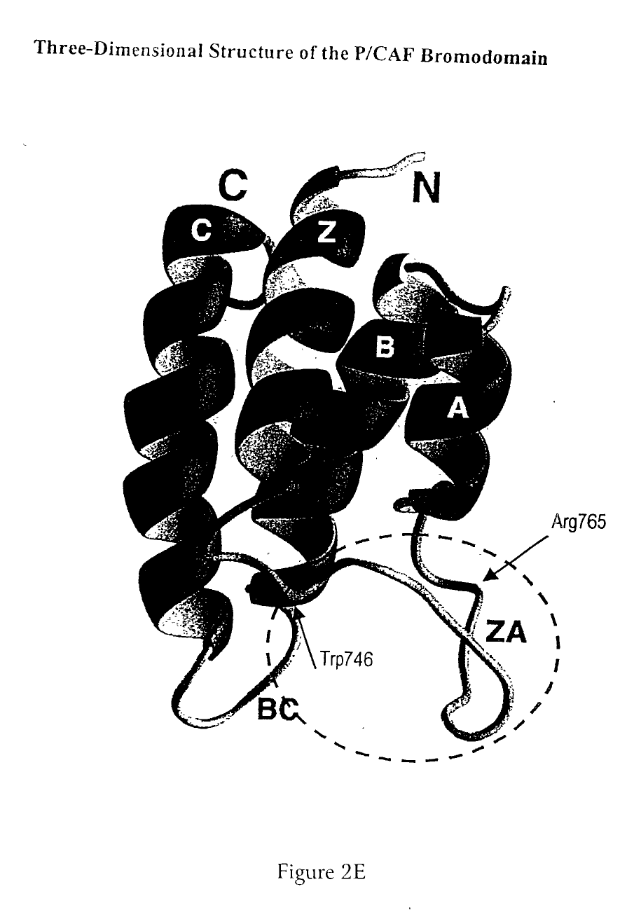

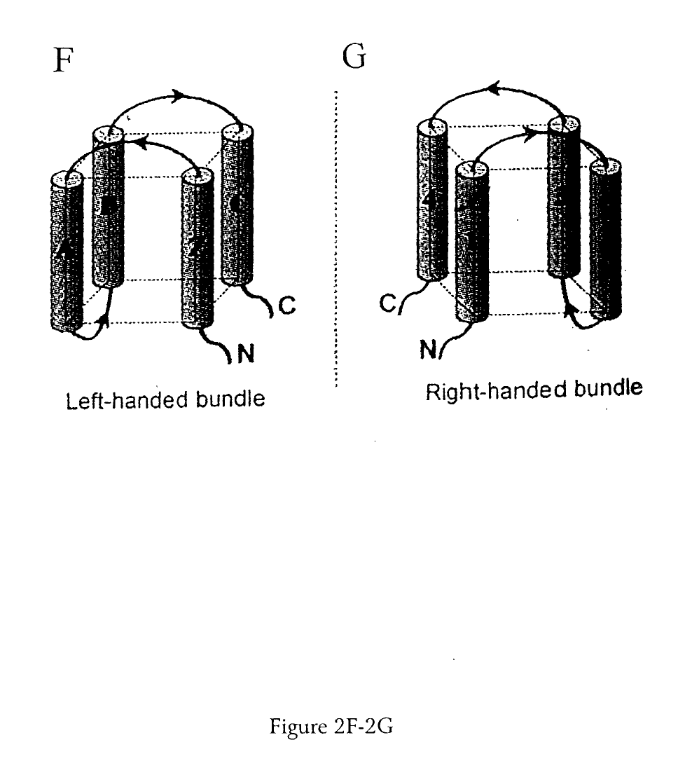

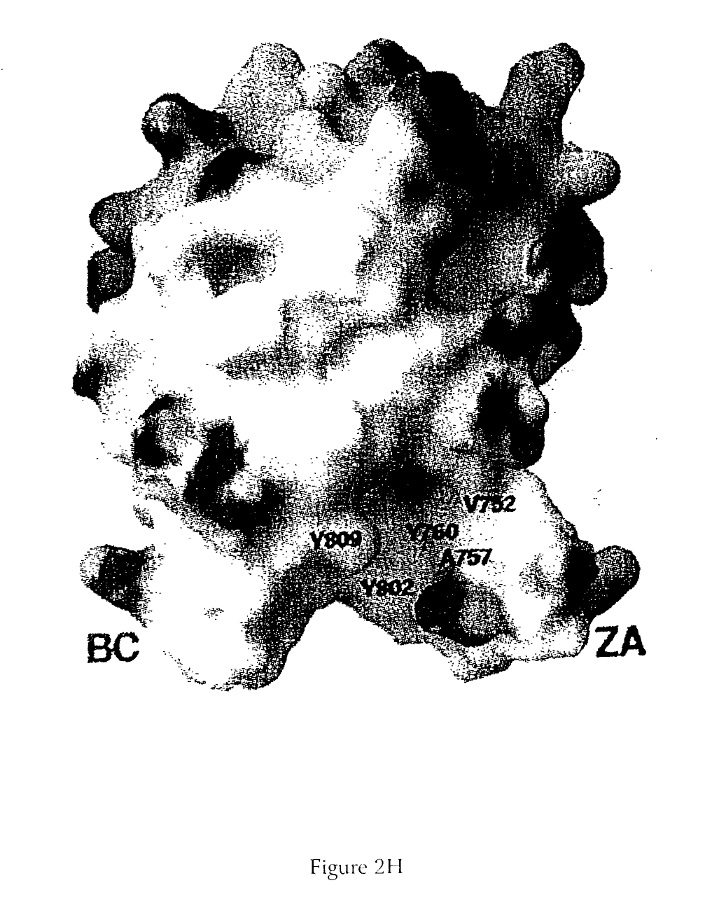

The present invention further provides a key region of the bromodomain for the interaction with its acetyl-lysine binding partner, the ZA loop. The amino acid sequence of the ZA loop is defined in FIG. 1 for a number of bromodomains and is depicted in FIG. 2A for P/CAF. In a particular embodiment, the ZA loop has between about 21 and 40 amino acid residues comprising the amino acid sequence:

F X₂₋₃P X₅₋₈J_{P/K/H}X Y J_{Y/F/H}X₅P J_{M/I/V}D (SEQ ID NO:3)

more preferably the ZA loop has about 23 to 34 amino acid residues and comprises the amino acid sequence:

X₂F X₂₋₃P X₅₋₈J_{P/K/H}X Y J_{Y/F/H}X₅P J_{M/I/V}D (SEQ ID NO:43)

In a specific embodiment, the ZA loop has between about 20 and 64 amino acid residues comprising the amino acid sequence:

F X₂₋₄V X₂₋₄E X₂₋₄Y X₁₋₃VJ_{I/L/M/V} (SEQ ID NO:48)

(1) The single letter amino acid code is used in this description, i.e.,“F” for phenylalanine; “P” for proline; “Y” for tyrosine; and “D” for aspartic acid.

(2) “X” indicates any amino acid (an undesignated amino acid); and X, X₂, X₂₋₃, X₅, and X₅₋₈ indicates one undesignated amino acid, two consecutive undesignated amino acids, two or three consecutive undesignated amino acids, five consecutive undesignated amino acids, and five to eight consecutive undesignated amino acids respectively.

(3) “J” indicates that identity of the amino acid is restricted to a particular group, again the one letter code is used:

(i) J_{P/K/H} is either proline, lysine or histidine.

(ii) J_{Y/F/H} is either tyrosine, phenylalanine or histidine.

(iii) J_{M/I/V} is either methionine, isoleucine, or valine.

(iv) J_{I/L/M/V} is either isoleucine, leucine, methionine, or valine

Since this region of the bromodomain is important in binding its acetyl-lysine binding partner, antibodies specifically raised against this region are also included in the present invention. In a particular embodiment, the antibody is a humanized chimeric antibody that can be used in therapeutic treatment. Thus monoclonal, chimeric, and polyclonal antibodies raised against bromodomains, preferably against amino acid residues in the ZA loop region are part of the present invention. In a specific embodiment the antibody is raised against a peptide, fusion peptide or conjugated peptide consisting of amino acid residues 746 to 765 of SEQ ID NO:2, i.e., WPFMEPVKRTEAPGYYEVIR (SEQ ID NO:44). In another embodiment the antibody is raised against a peptide, fusion peptide or conjugated peptide consisting of amino acid residues 748 to 809 of SEQ ID NO:2 (which is SEQ ID NO:49). Such antibodies can be used in the treatment of leukemia or AIDs for example. Alternatively, these antibodies can be used in drug discovery assays.

Analogously, the present invention provides peptides derived from the HIV-1 Tat protein. In one such embodiment the peptide comprises 7 to 21 amino acid residues comprising the amino acid sequence

YGRKX₁₋₃RQ (SEQ ID NO:46)

In a specific embodiment the peptide fragment of Tat has ten amino acid residues and the amino acid sequence:

SYGRKKRRQR (SEQ ID NO:47)

Preferably the lysine corresponding to lysine50 of Tat (see SEQ ID NO:45) is acetylated. These peptide fragments can be used in the drug assays of the present invention and/or as antigens for antibodies that specifically interfere with the interaction (e.g., binding) of Tat with P/CAF interaction.

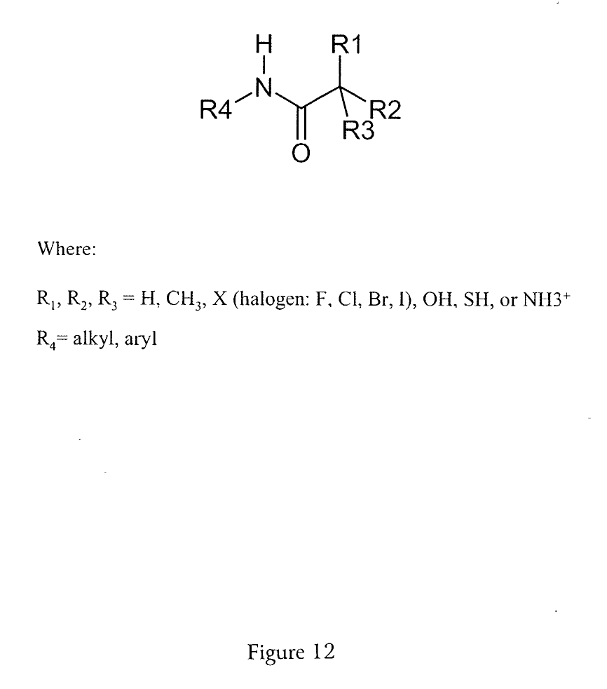

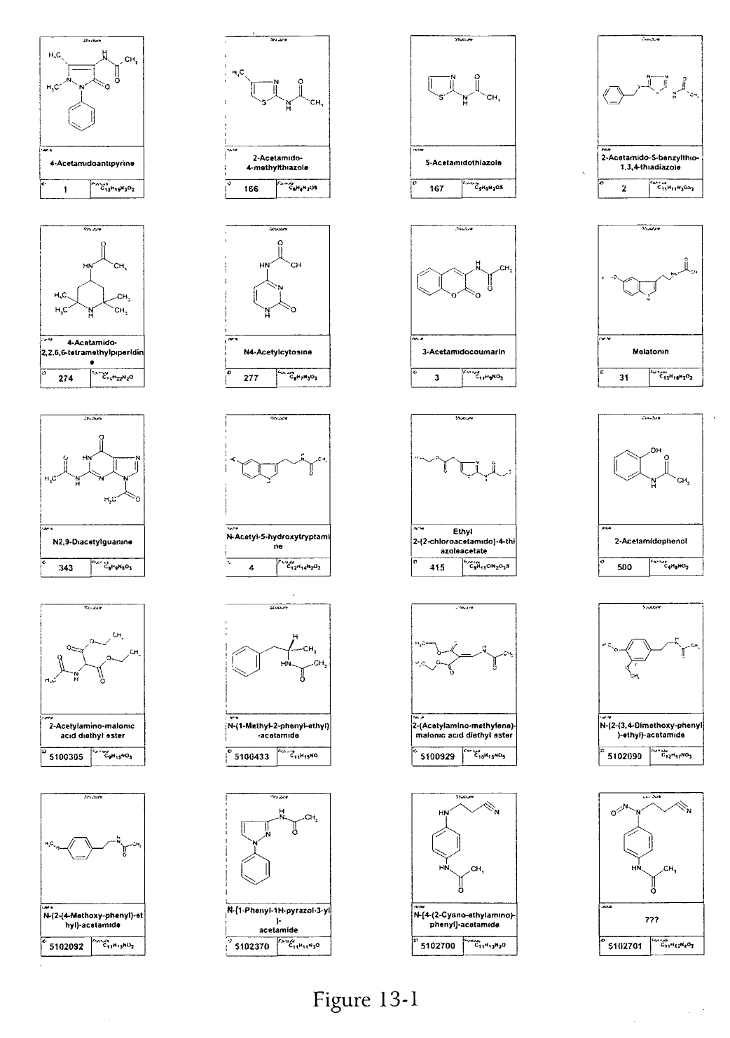

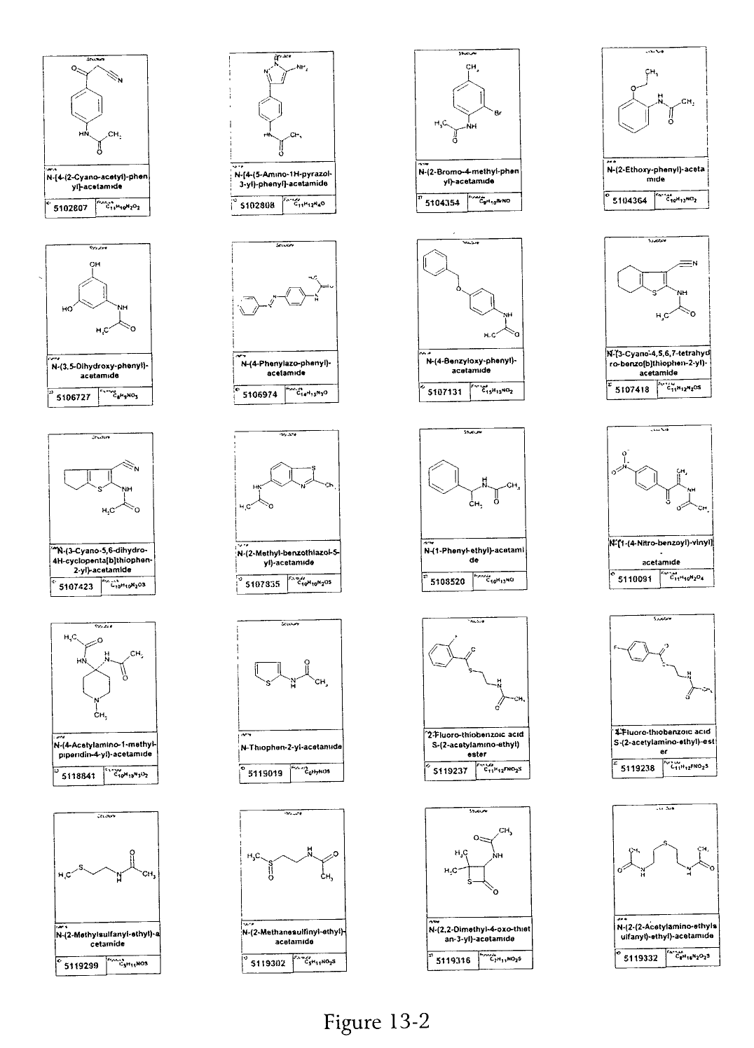

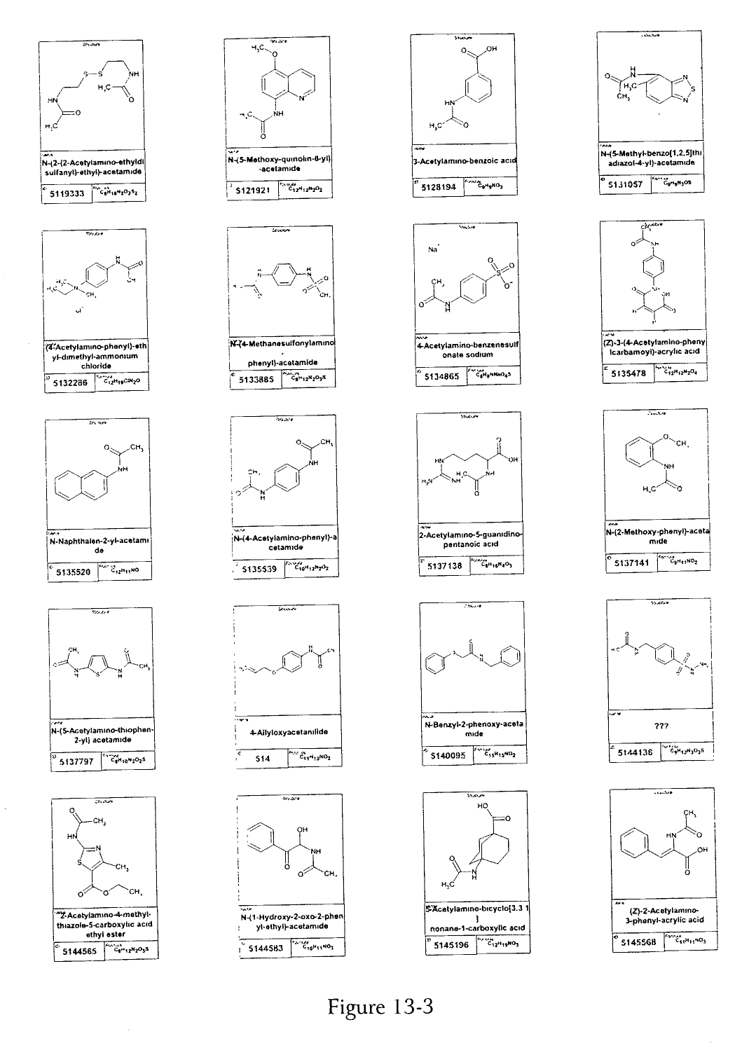

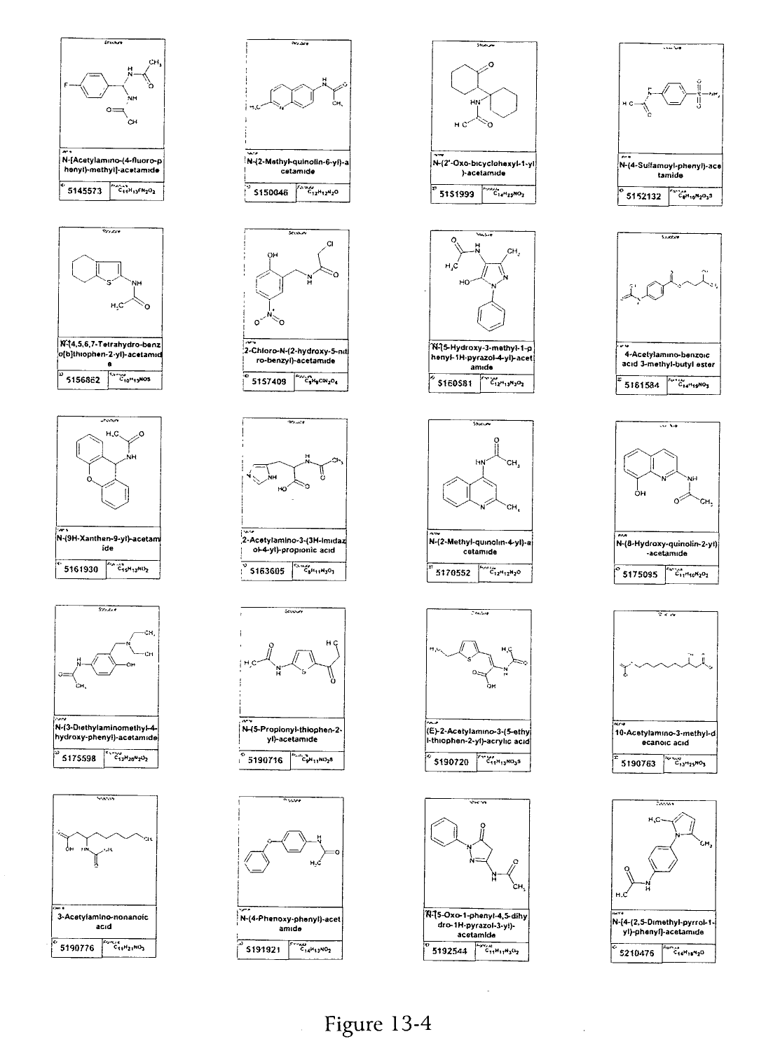

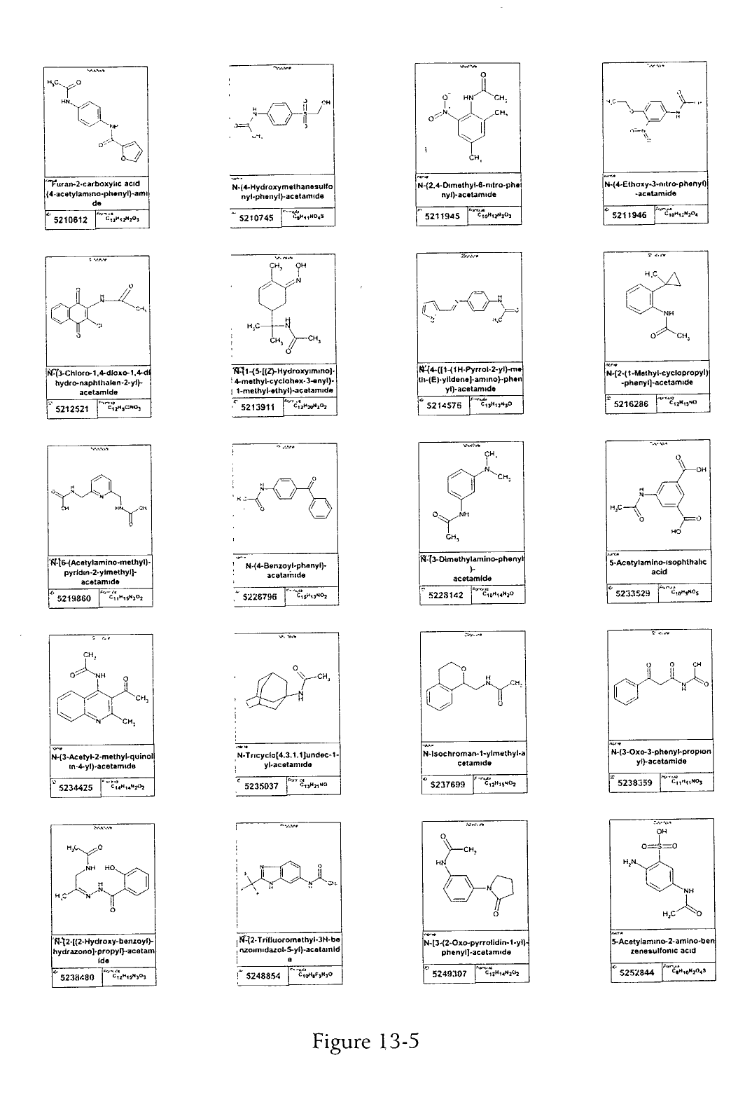

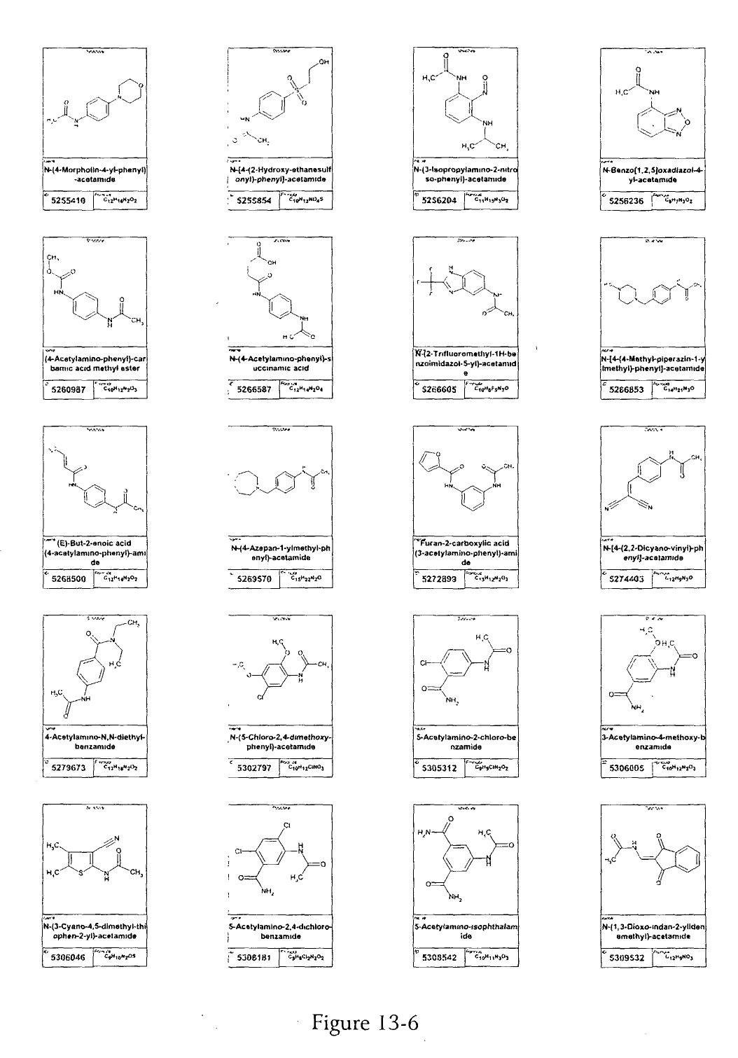

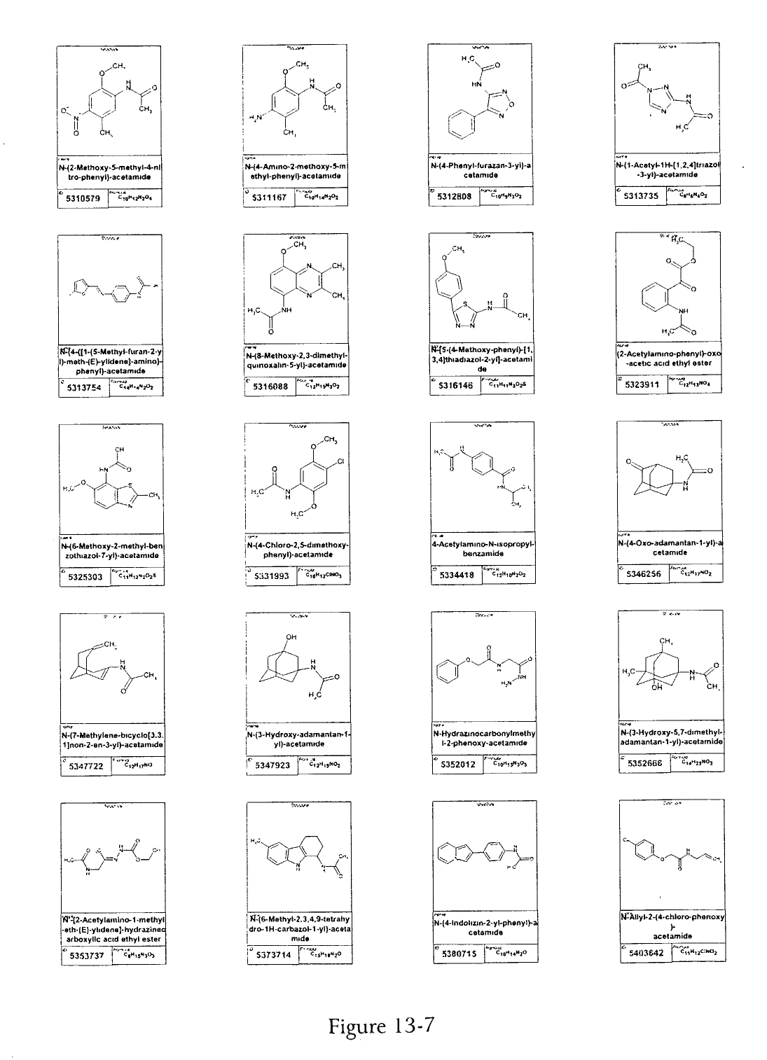

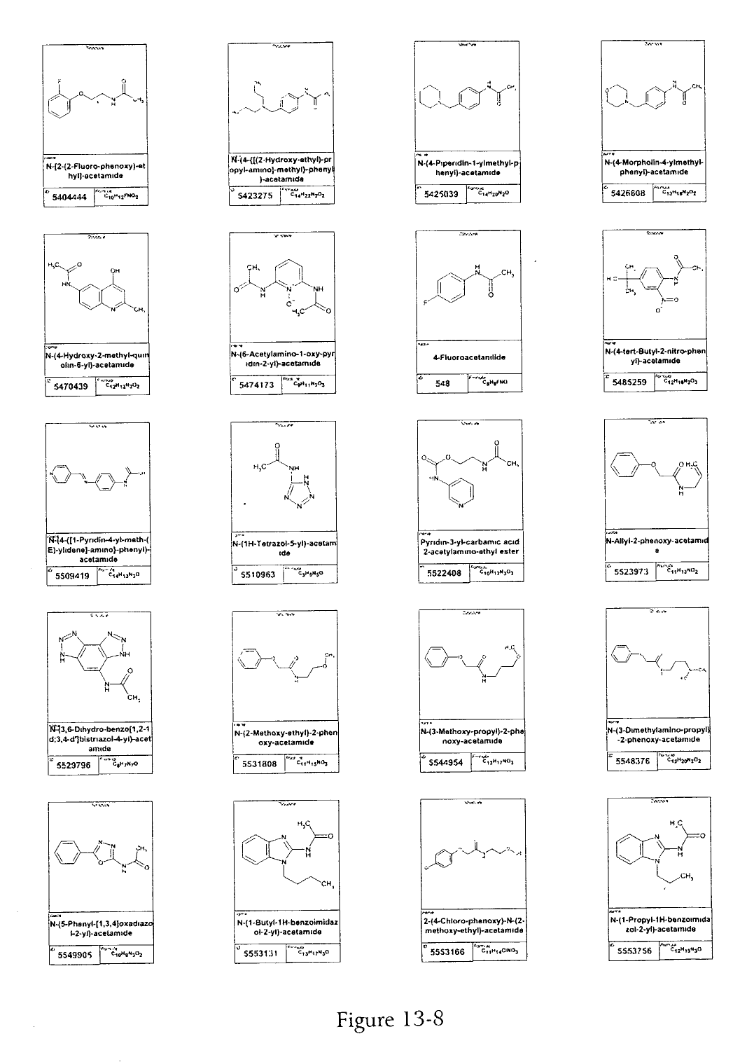

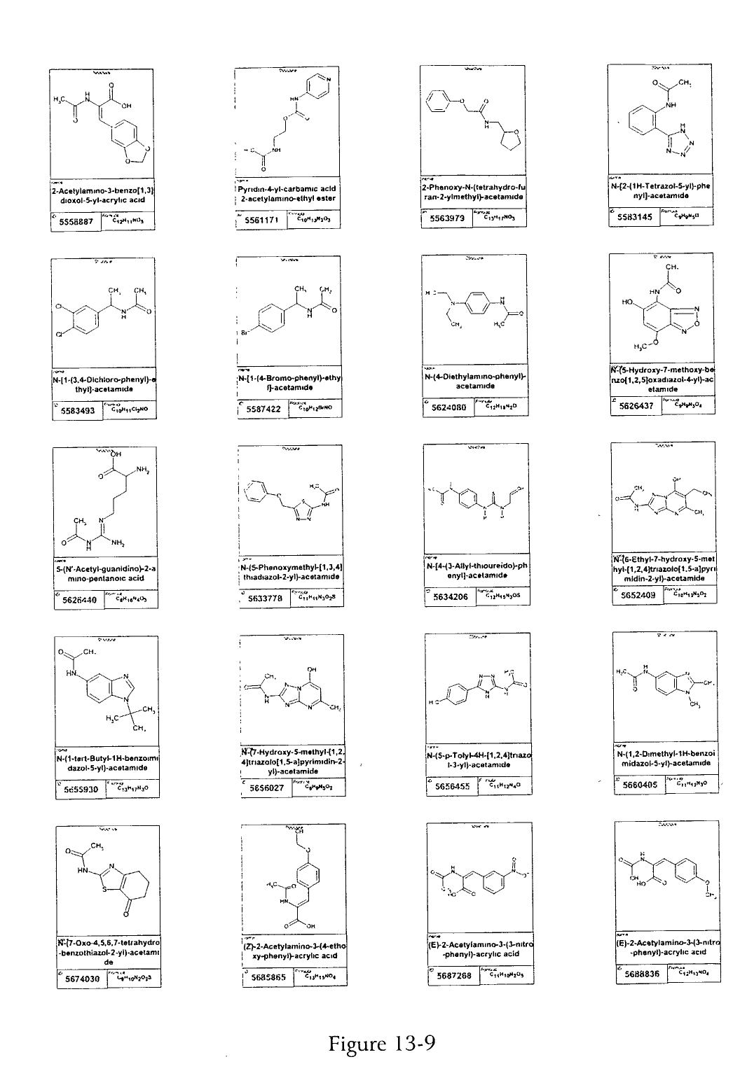

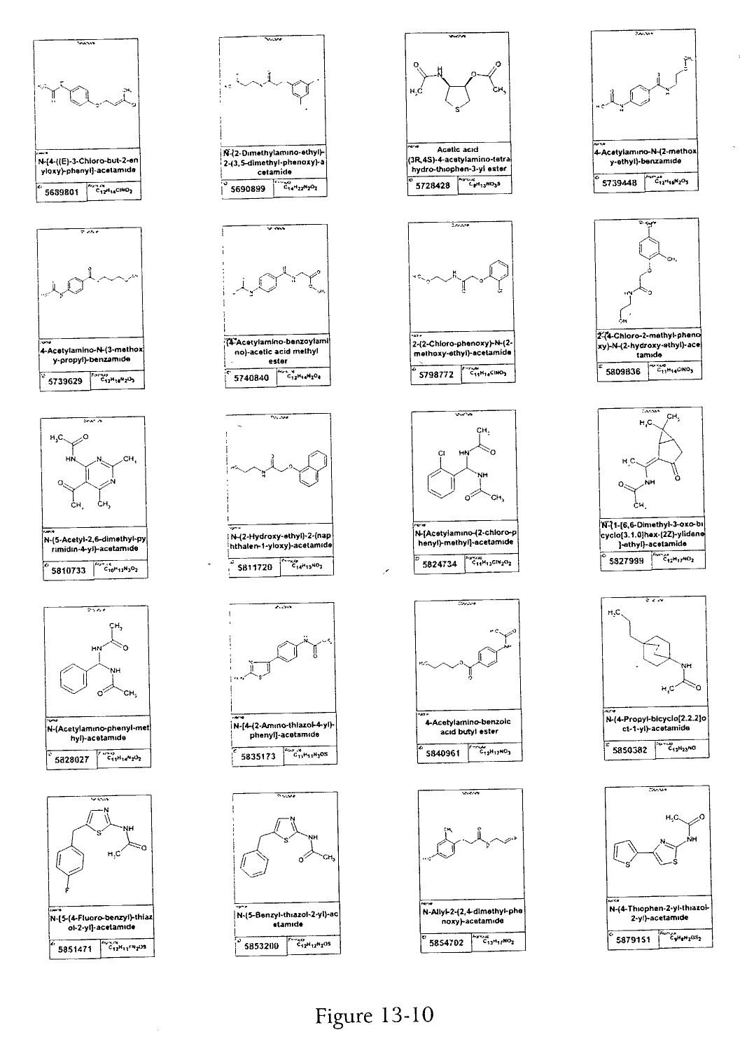

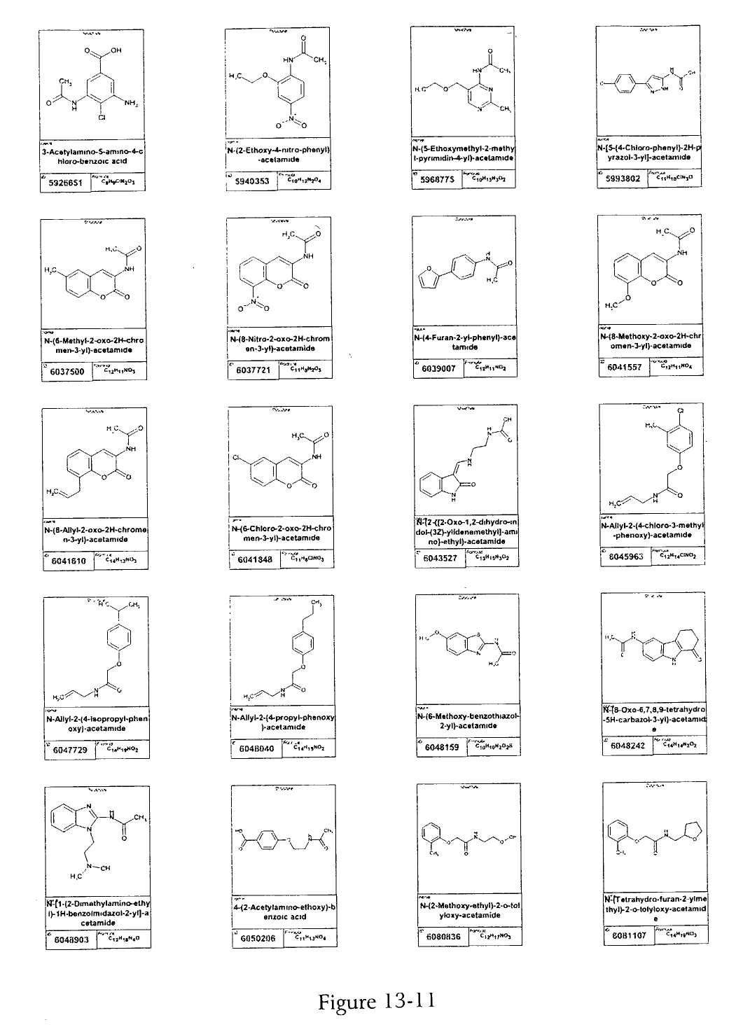

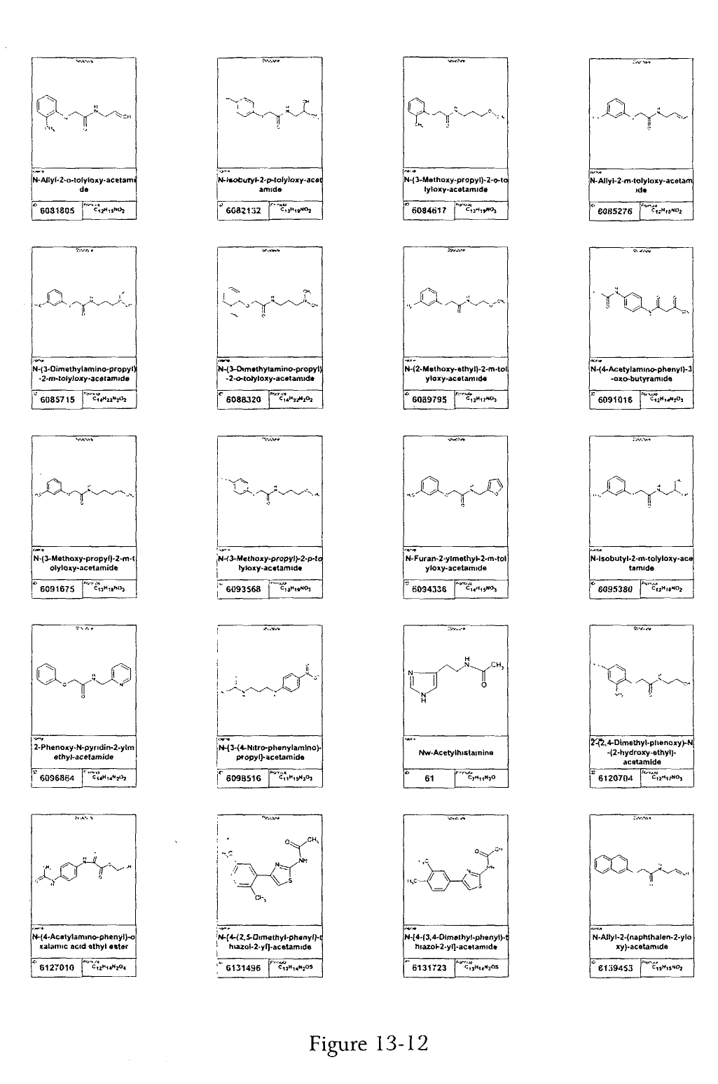

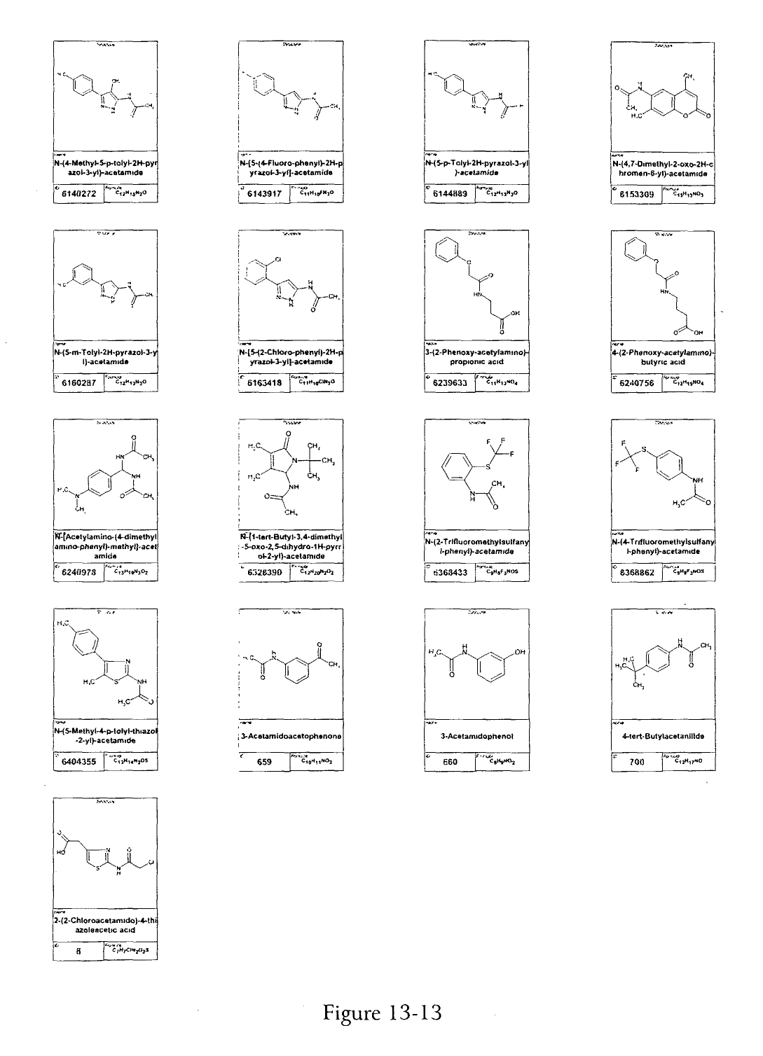

The present invention provides the first detailed structural information regarding a bromodomain and a bromodomain complexed with its acetylated binding partner. The present invention therefore provides the three-dimensional structure of the bromodomain and a bromodomain acetylated binding partner complex. Since the interaction of the bromodomain with a histone for example, can play a significant role in chromatin remodeling/regulation, the structural information provided herein can be employed in methods of identifying drugs that can modulate basic cell processes by modulating the transcription. In a particular embodiment, the three-dimensional structural information is used in the design of a small organic molecule for the treatment of cancer or as disclosed below, HIV-1 infection and/or AIDs. In addition, the present invention provides a critical structural feature for a class of inhibitors (acetyl-lysine analogs) of the interaction between bromodomains and their protein binding partners which contain an acetylated-lysine (e.g., Tat with P/CAF), see FIG. 12, as well as a compilation of compounds that share this critical feature, see FIG. 13.

Indeed, the bromodomain and lysine-acetylated protein interaction can now be implicated to play a causal role in the development of a number of diseases including cancers such as leukemia. For example, chromatin remodeling plays a central role in the etiology of viral infection and cancer [Archer and Hodin, Curr. Opin. Genet. Biol. 9:171-174 (1999); Jacobson and Pillus, Curr. Opin. Genet. Biol. 9:175-184 (1999)]. Both altered histone acetylation/deacetylation and aberrant forms of chromatin-remodeling complexes are associated with human diseases. Furthermore, chromosomal translocation of various cellular genes with those encoding HATs and subunits of chromatin remodeling complexes have been implicated in leukomogenesis. The MOZ (monocytic leukemia zinc finger) and MLL/ALL-1 genes are frequently fused to the gene encoding the co-activator HAT CBP [Sobulo et al., Proc. Natl. Acad. Sci. USA 94:8732-8737(1997)]. The resulting fusion protein MLL-CBP contains the tandem bromodomain-PHD finger-HAT domain of CBP. It also has been shown that both the bromodomain and HAT domain of CBP are required for leukomogenesis, because deletion of either the bromodomain or the HAT domain results in loss of the MLL-CBP fusion protein's ability for cell transform. These results indicate that the CBP bromodomain, and more particularly, the ZA loop of the CBP bromodomain, is an excellent target for developing drugs that interfere with the bromodomain acetyl-lysine interaction that can be used in the treatment of human acute leukemia. In addition, an antibody (e.g., a humanized antibody) raised specifically against a peptide from the ZA loop of the CBP bromodomain could also be effective for treating these conditions.

In addition, it now known that the human immunodeficiency virus type 1 (HIV-1) trans-activator protein, Tat, is tightly regulated by lysine acetylation [Kiernan et al., EMBO Journal 18:6106-6118 (1999)]. HIV-1 Tat transcriptional activity is absolutely required for productive HIV viral replication [Jeang and Gatignol, Curr. Top. Microbiol. Immunol., 188:123-144(1994)]. Therefore, the interaction of the acetyl-lysine of Tat with one or more bromodomain-containing proteins associated with chromatin remodeling could mediate gene transcription. More particularly, it is disclosed herein that acetylated lysine50 of Tat specifically binds to the bromodomain of P/CAF. Therefore, this particular bromodomain/lysine-acetylated Tat interaction serves as a drug target for blocking HIV replication in cells. As indicated above, an antibody raised specifically against a peptide from the ZA loop of the P/CALF bromodomain could also be effective for treating and/or preventing HIV infections including those that lead to AIDs.

In addition, based on the new structural information disclosed herein, the key amino acid residues for the binding of a given bromodomain and its binding partner can be identified and further elucidated using basic mutagenesis and standard isothermal titration calorimetry, for example. Indeed, both the critical amino acids for the bromodomain and the binding partner (i.e., apart from the acetyl-lysine) can be readily determined and are also part of the present invention.

Therefore, the results obtained from the structural and functional studies disclosed herein provide the foundation for both high throughput drug screening and structure-based rational drug design. The agents identified by this procedure are useful for ameliorating conditions involving chromatin remodeling/regulation, and/or in the treatment of cancer and/or AIDS, as indicated above.

Structure based rational drug design is the most efficient method of drug development. However, heretofore, no information has been disclosed regarding the structure of the bromodomain or more importantly, its interaction with the acetyl-lysine of its binding partner. Obtaining detailed structural information requires an extensive NMR or X-ray crystallographic analysis. By determining and then exploiting the detailed structural information of the bromodomain and of the bromodomain/acetyl-histamine (exemplified by NMR analysis below) the present invention provides novel methods for developing new drugs through structure based rational drug design.

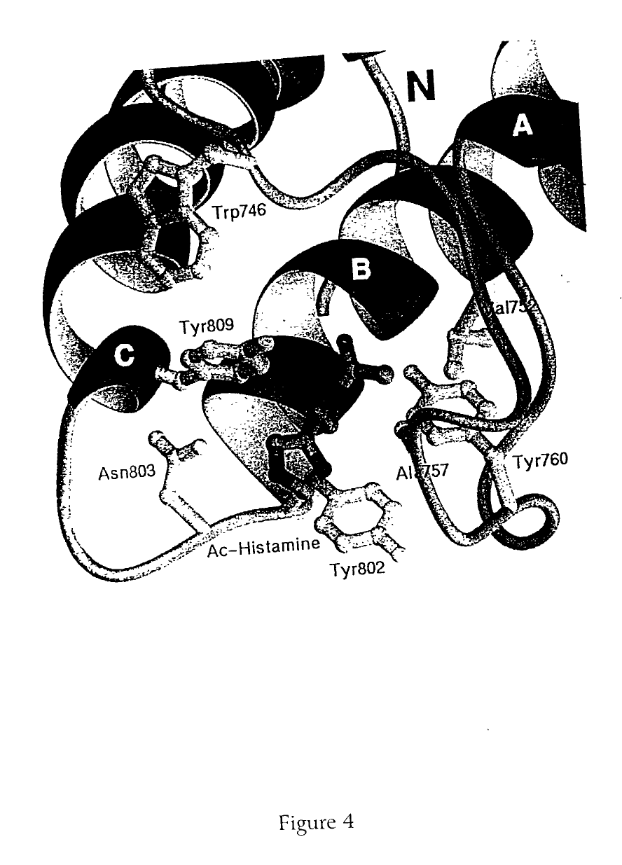

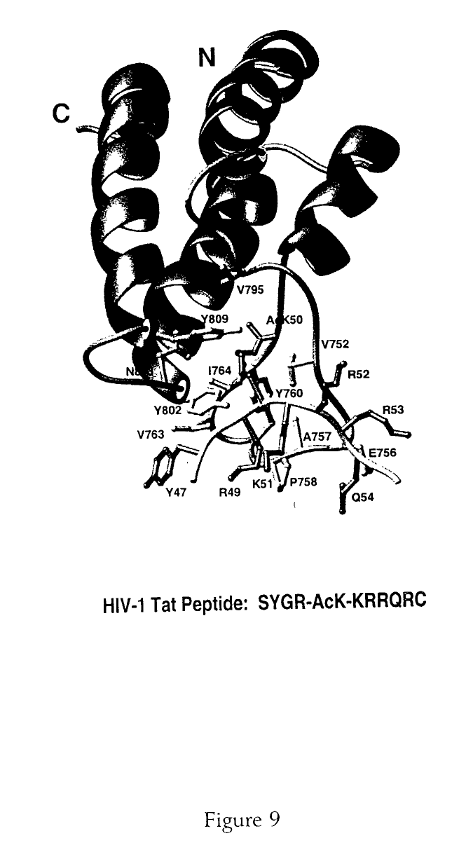

Thus the present invention provides representative sets of the atomic structure coordinates of the free form of the P/CAF bromodomain (Table 5), of the P/CAF bromodomain-acetyl-histamine complex (Table 6) and of the Tat-P/CAF complex (Table 10) which were all obtained by NMR analysis. A Ribbon diagram of the three-dimensional structure of the P/CAF bromodomain is depicted in FIG. 2E, whereas the P/CAF bromodomain acetyl-lysine binding pocket is depicted in FIG. 4 and the Tat-P/CAF complex is depicted in FIG. 9. The present invention also provides the NOE-derived distance restraints, and NMR chemical shift assignments of the P/CAF bromodomain, and the Tat-P/CAF complex. The NMR chemical shift assignments of the P/CAF bromodomain are included in the chemical shift table (Table 1) for the ¹H-¹⁵N HSQC spectrum of P/CAF bromodomain. The unambiguous NOE-derived Inter-proton Distance Restraints (Table 2), the ambiguous NOE-derived Inter-proton Distance Restraints (Table 3) and the ¹H bonding restraints (Table 4) are also disclosed herein. The NMR chemical shift assignments of the Tat-P/CAF complex are included in the chemical shift table (Table 11) for the ¹H-¹⁵N HSQC spectrum of P/CAF bromodomain. The unambiguous NOE-derived Inter-proton Distance Restraints (Table 13), the ambiguous NOE-derived Inter-proton Distance Restraints (Table 14) and the ¹H bonding restraints (Table 12) are also disclosed herein. The sample atomic coordinate data provided enable the skilled artisan to practice the invention.

In addition, Tables 1-6 and/or 10-14 are also capable of being placed into a computer readable form which is also part of the present invention. Furthermore, methods of using these coordinates and chemical shifts and related information (including in computer readable forms) either individually or together in drug assays are also provided. More particularly, such atomic coordinates can be used to identify potential ligands or drugs which will modulate the binding of a bromodomain with its binding partner.

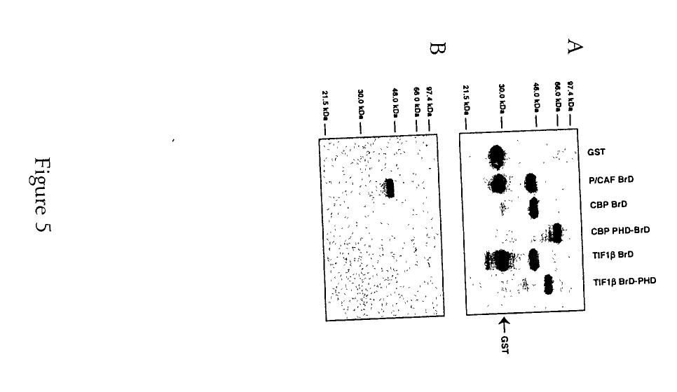

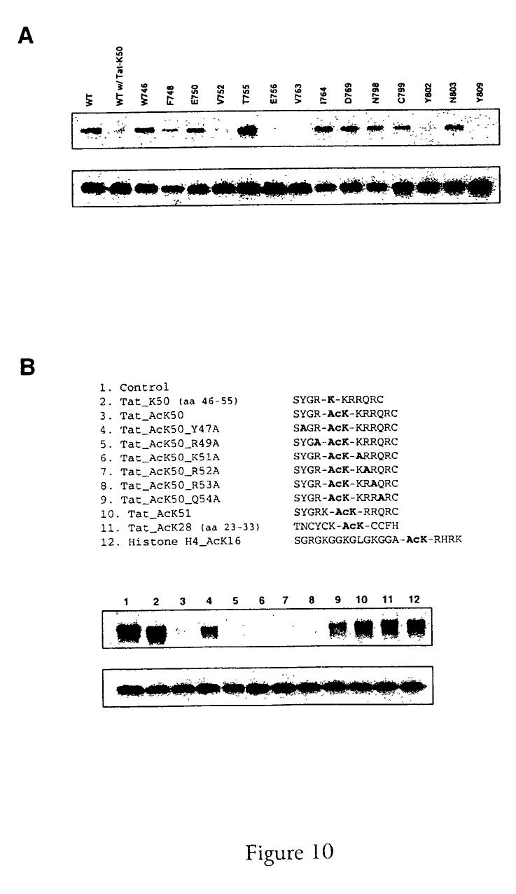

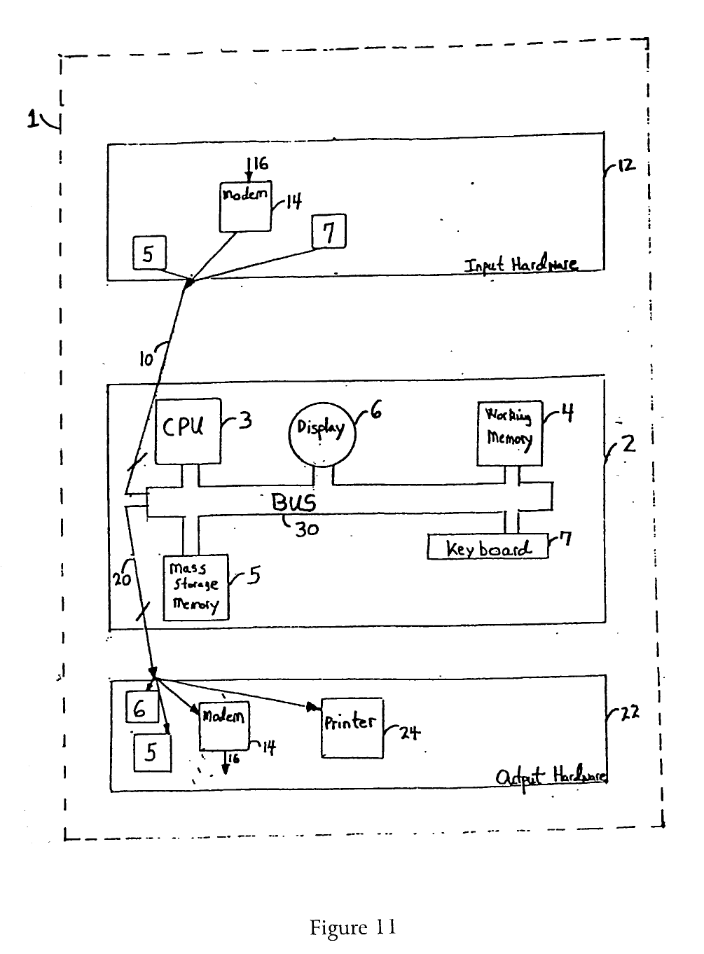

In a particular aspect of the present invention, the lysine-acetylated Tat is shown herein to specifically bind to the bromodomain of the p300/CBP-associated factor (P/CAF) in vitro and in vivo. Structural and mutational analyses provides the identification of key amino acid residues on both the bromodomain and Tat that are important for the binding complex. The identification of these important amino acid residues further demonstrates the biological importance of this interaction for Tat transactivation activity. Together, the findings disclosed herein indicate a novel mechanism by which the lysine-acetylated Tat recruits P/CAF via a bromodomain interaction, leading to chromatin remodeling-mediated transcriptional activation of HIV-1. Furthermore, the extreme specificity of the Tat-P/CAF binding (see e.g., FIGS. 5A-5B and 10A-10B) indicates that compounds that interfere with this binding complex are not likely to interfere to otherwise related bromodomain-ligand interactions.

Therefore, the three-dimensional structural information provided by the present invention allows the identification and/or design of specific compounds that can act as modulators of crucial processes. In the case of the Tat-P/CAF interaction, such compounds can be used as drugs to inhibit HIV-1 expression in a cell and/or subsequent infection of other cells. Therefore, the inhibitors identified and/or designed by the methods disclosed can be used to prevent, treat, retard the progression, and potentially cure HIV-1 infections and AIDS.

Definitions

As used herein a “bromodomain-acetyl-lysine binding complex” is a binding complex between a bromodomain or fragment thereof and either a peptide/polypeptide comprising an acetyl-lysine (or an analog of acetyl-lysine), or a free analog of acetyl-lysine, such as acetyl-histamine disclosed in the Example below. Preferably, the peptide comprises at least six amino acids in addition to the acetyl-lysine. A fragment of a bromodomain preferably comprises a ZA loop as defined below. The dissociation constant of a bromodomain-acetyl-lysine binding complex is dependent on whether the lysine residue or analog thereof is acetylated or not, such that the affinity for the bromodomain and the peptide comprising the lysine residue (for example) significantly decreases when that lysine residue is not acetylated. One example of a bromodomain-acetyl-lysine binding complex is that formed between P/CAF with Tat (the “Tat-P/CAF complex”) as exemplified below.

As used herein the term “acetyl-lysine analog” is used interchangeably with the term “analog of acetyl-lysine” and is a compound that contains the acetyl-amine-like structure as depicted in FIG. 12. Examples of acetyl-lysine analogs are included in FIG. 13.

As used herein a “ZA loop” of a bromodomain is a key protion of a bromodomain that is involved in the binding of the bromodomain to the acetyl-lysine. The structure of the actual ZA loop of the bromodomain of P/CAF is depicted in FIG. 2A. As used herein, however, a ZA loop has between about 20 and 40 amino acids and preferably comprises the amino acid sequence of SEQ ID NO:3 and/or SEQ ID NO:48. More preferably the ZA loop comprises between about 23 to 34 amino acids. In a specific embodiment the ZA loop has the amino acid sequence SEQ ID NO:43. The amino acid sequence of the ZA loop for a representative number of individual bromodomains is shown in FIG. 1.

A “polypeptide” or “peptide” comprising a fragment of a bromodomain, such as the ZA loop, or a peptide or polypeptide comprising an acetyl-lysine, as used herein can be the “fragment” alone, or a larger chimeric or fusion, peptide/protein which contains the “fragment”.

As used herein the terms “fusion protein” and “fusion peptide” are used interchangeably and encompass “chimeric proteins and/or chimeric peptides” and fusion “intein proteins/peptides”. A fusion protein comprises at least a portion of a protein or peptide of the present invention, e.g., a bromodomain, joined via a peptide bond to at least a portion of another protein or peptide including e.g., a second bromodomain in a chimeric fusion protein. In a particular embodiment the portion of the bromodomain is antigenic. Fusion proteins can comprise a marker protein or peptide, or a protein or peptide that aids in the isolation and/or purification of the protein, for example.

As used herein, and unless otherwise specified, the terms “agent”, “potential drug”, “compound”, “test compound” or “potential compound” are used interchangeably, and refer to chemicals which potentially have a use as an inhibitor or activator/stabilizer of bromodomain-acetyl-lysine binding. Therefore, such “agents”, “potential drugs”, “compounds” and “potential compounds” may be used, as described herein, in drug assays and drug screens and the like.

As used herein a “small organic molecule” is an organic compound, including a peptide [or organic compound complexed with an inorganic compound (e.g., metal)] that has a molecular weight of less than 3 Kilodaltons. Such small organic molecules can be included as agents, etc. as defined above.

As used herein the term “binds to” is meant to include all such specific interactions that result in two or more molecules showing a preference for one another relative to some third molecule. This includes processes such as covalent, ionic, hydrophobic and hydrogen bonding but does not include non-specific associations such as solvent preferences.

General Techniques for Constructing Nucleic Acids that Encode the Bromodomains and Fragments Thereof (Incuding, ZA Loops); and the Bromodomain Binding Partners of the Present Invention.

In accordance with the present invention there may be employed conventional molecular biology, microbiology, and recombinant DNA techniques within the skill of the art. Such techniques are explained fully in the literature. See, e.g., Sambrook and Russell Molecular Cloning: A Laboratory Manual, Third Edition (2001) Vols. I-III, Cold Spring Harbor Laboratory Press, Cold Spring Harbor, N.Y. (herein “Sambrook and Russell, 2001”), Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength [see Sambrook et al., 1989 supra, Sambrook and Russell, 2001]. The conditions of temperature and ionic strength determine the “stringency” of the hybridization. For preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived [see Sambrook et al., 1989 supra, 9.50-10.51, Sambrook and Russell, 2001]. For hybridization with shorter nucleic acids, i.e., oligonucleotides, the position of mismatches becomes more important, and the length of the oligonucleotide determines its specificity [see Sambrook et al., 1989 supra, 11.7-11.8, Sambrook and Russell, 2001]. Preferably a minimum length for a hybridizable nucleic acid is at least about 12 nucleotides; preferably at least about 18 nucleotides; and more preferably the length is at least about 27 nucleotides; and most preferably 36 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

As used herein, the term “homologous” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) [Reeck et al., Cell, 50:667 (1987)]. Such proteins have sequence homology as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and not a common evolutionary origin.

Two DNA sequences are “substantially homologous” when at least about 60% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art [See, e.g., Sambrook et al., 1989 supra; DNA Cloning, Vols. I & II, supra; Nucleic Acid Hybridization, supra., and Sambrook and Russell, 2001.]

As used herein an amino acid sequence is 100% “homologous” to a second amino acid sequence if the two amino acid sequences are identical, and/or differ only by neutral or conservative substitutions as defined below. Accordingly, an amino acid sequence is 50% “homologous” to a second amino acid sequence if 50% of the two amino acid sequences are identical, and/or differ only by neutral or conservative substitutions. As used herein, DNA and protein sequence percent identity can be determined using MacVector 6.0.1, Oxford Molecular Group PLC (1996) and the Clustal W algorithm with the alignment default parameters, and default parameters for identity. These commercially available programs can also be used to determine sequence similarity using the same or analogous default parameters.

The present invention relates to cloning vectors containing nucleic acids encoding analogs and derivatives of the bromodomains of the present invention and polypeptides/peptides that can bind a bromodomain when a lysine of the polypeptide/peptide is acetylated, including modified fragments, that have the same or homologous functional activity as the individual fragments, and homologs thereof. The production and use of derivatives and analogs related to the fragments are within the scope of the present invention.

Due to the degeneracy of nucleotide coding sequences, other DNA sequences which encode substantially the same amino acid sequence as a nucleic acid encoding a protein comprising bromodomain or bromodomain binding partner (i.e., when post-transcriptionally acetylated) of the present invention for example, may be used in the practice of the present invention. These include but are not limited to allelic genes, homologous genes from other species, which are altered by the substitution of different codons that encode the same amino acid residue within the sequence, thus producing a silent change. Likewise, the peptides and polypeptides of the present invention include, but are not limited to, those containing, as a primary amino acid sequence, analogous portions of their respective amino acid sequences including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, and lysine. The negatively charged (acidic) amino acids include aspartic acid and glutamic acid.

All of the peptides/fragments of the present invention can be modified by being placed in a fusion or chimeric peptide or protein, or labeled e.g., to have an N-terminal FLAG-tag, or H6 tag. In a particular embodiment the P/CAF bromodomain fragment can be modified to contain a marker protein such as green fluorescent protein as described in U.S. Pat. No. 5,625,048 and WO 97/26333, each of which are hereby incorporated by reference herein in their entireties.

The nucleic acids encoding peptides and protein fragments of the present invention and analogs thereof can be produced by various methods known in the art. The manipulations which result in their production can occur at the gene or protein level [Sambrook et al., 1989, supra; Sambrook and Russell, 2001, supra]. The nucleotide sequence can be cleaved at appropriate sites with restriction endonuclease(s), followed by further enzymatic modification if desired, isolated, and ligated in vitro. In addition a nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis [Hutchinson et al., J. Biol. Chem., 253:6551 (1978); Zoller and Smith, DNA, 3:479-488 (1984); Oliphant et al., Gene, 44:177 (1986); Hutchinson et al., Proc. Natl. Acad. Sci. U.S.A., 83:710 (1986)], use of TAB® linkers (Pharmacia), etc. PCR techniques are preferred for site directed mutagenesis [see Higuchi,“Using PCR to Engineer DNA”, in PCR Technology: Principles and Applications for DNA Amplification, H. Erlich, ed., Stockton Press, Chapter 6, pp. 61-70 (1989)].

Protein Expression and Purification

A bacterial protein expression system can be used to make various stable isotopically labeled (¹³C, ¹⁵N, and ²H) protein samples that are useful for a three-dimensional NMR structural determination of a protein complex. For example a pET14b (Novagen) bacterial expression vector can be constructed which expresses the recombinant P/CAF bromodomain as an amino-terminal His-tagged fusion protein.

Protein expression and purification can be conducted using standard procedures for His-tagged proteins [Zhou et al., J. Biol. Chem. 270:31119-31123 (1995)]. To optimize the level of protein expression, various bacterial growth and expression conditions can be screened, which include different E. Coli cell lines, and growth and protein induction temperatures. Generally, it is preferred to obtain the maximum amount of soluble protein while still inducing protein expression with a relatively low IPTG concentration e.g., ˜0.2 mM (final concentration) at 16° C. As exemplified below, the bromodomain of P/CAF (residues 719-832 of SEQ ID NO:2 which is SEQ ID NO:7) was subcloned into the pET14b expression vector (Novagen) and expressed in Escherichia coli BL21(DE3) cells. Uniformly ¹⁵N- and ¹⁵N/¹³C-labeled proteins were prepared by growing bacteria in a minimal medium containing ¹⁵NH₄Cl with or without ¹³C₆-glucose. A uniformly ¹⁵N/¹³C-labeled and fractionally deuterated protein sample was prepared by growing the cells in 75% ²H₂O. The bromodomain was purified by affinity chromatography on a nickel-IDA column (Invitrogen) followed by the removal of poly-His tag by thrombin cleavage. The final purification of the protein was achieved by size-exclusion chromatography. The acetyl-lysine-containing peptides were prepared on a MilliGen 9050 peptide synthesizer (Perkin Elmer) using Fmoc/HBTU chemistry. Acetyl-lysine was incorporated using the reagent Fmoc-Ac-Lys with HBTU/DIPEA activation. NMR samples contained approximately 1 mM protein in 100 mM phosphate buffer of pH 6.5 and 5mM perdeuterated DTT and 0.5 mM EDTA in H₂O/²H₂O (9/1) or ²H₂O.

One major advantage of using the heteronuclear multidimensional approach, as exemplied herein, is that the NMR resonance assignments of a protein are obtained in a sequence-specific manner which assures accuracy and greatly facilitates data analysis and structure determination [Clore and Gronenborn Meth. Enzymol. 239:249-363 (1994)]. In addition, the signal overlapping problems in the protein spectra are minimized by the use of multidimensional NMR spectra, which separates the proton signals according to the chemical shifts of their attached hetero-nuclei (such as ¹⁵N and ¹³C). This NMR approach has been proven very powerful for structural analysis of large proteins [Clore and Gronenborn Meth. Enzymol. 239:249-363 (1994)]. To facilitate sequence-specific resonance assignments for the structural study, a uniformly ¹³C, ¹⁵N-labeled and fractionally (75%) deuterated protein sample of the bromodomain can be prepared by growing bacterial cells in 75% ²H₂O as exemplified below. Such protein samples can be used for triple-resonance NMR experiments. A triple-labeled protein sample is useful for high-resolution NMR structural studies. Because of the favorable ¹H, ¹³C, and ¹⁵N relaxation rates caused by the partial deuteration of the protein, constant-time triple-resonance NMR spectra can be acquired with higher digital resolution and sensitivity [Sattler, M. & Fesik, S. W. Structure 4:1245-1249 (1996)]. In addition, various stable-isotopically labeled (¹⁵N and ¹³C /¹⁵N) proteins can also be prepared using this procedure.

Synthetic Polypeptides

The term “polypeptide” is used in its broadest sense to refer to a compound of two or more subunit amino acids, amino acid analogs, or peptidomimetics. The subunits are linked by peptide bonds. The terms “polypeptide”, “protein”, and “peptide” are used interchangeably herein, though preferably as used herein a “peptide” refers to a compound of at least two but less than fifty subunit amino acids, and a polypeptide or protein refers to compound of fifty or more amino acids. The polypeptides of the present invention may be chemically synthesized or as detailed above, genetically engineered or isolated from natural sources.

In addition, potential drugs or agents that may be tested in the drug screening assays of the present invention may also be chemically synthesized. When the peptide is to be modified, e.g., acetylated, the modification can be at any time during the peptide synthesis, including using an acetyl-lysine as a starting material or acetylating a lysine residue of a peptide after the peptide has been synthesized. In the Example below, the acetyl-lysine-containing peptides were prepared on a MilliGen 9050 peptide synthesizer (Perkin Elmer) using Fmoc/HBTU chemistry. Acetyl-lysine was incorporated using the reagent Fmoc-Ac-Lys with HBTU/DIPEA activation.

Thus, synthetic polypeptides, prepared using the well known techniques of solid phase, liquid phase, or peptide condensation techniques, or any combination thereof, can include natural and unnatural amino acids. Amino acids used for peptide synthesis may be standard Boc (N^{α}-amino protected N^{α}-t-butyloxycarbonyl) amino acid resin with the standard deprotecting, neutralization, coupling and wash protocols of the original solid phase procedure of Merrifield [J. Am. Chem. Soc., 85:2149-2154 (1963)], or the base-labile N^{α}-amino protected 9-fluorenylmethoxycarbonyl (Fmoc) amino acids first described by Carpino and Han [J. Org. Chem., 37:3403-3409 (1972)]. Both Fmoc and Boc N^{α}-amino protected amino acids can be obtained from Fluka, Bachem, Advanced Chemtech, Sigma, Cambridge Research Biochemical, Bachem, or Peninsula Labs or other chemical companies familiar to those who practice this art. In addition, the method of the invention can be used with other N^{α}-protecting groups that are familiar to those skilled in this art. Solid phase peptide synthesis may be accomplished by techniques familiar to those in the art and provided, for example, in Stewart and Young [Solid Phase Synthesis, Second Edition, Pierce Chemical Co., Rockford, Ill. (1984)] and Fields and Noble [Int. J. Pept. Protein Res., 35:161-214 (1990)], or using automated synthesizers, such as sold by ABS. Thus, polypeptides of the invention may comprise D-amino acids, a combination of D- and L-amino acids, and various “designer” amino acids (e.g., β-methyl amino acids, Cα-methyl amino acids, and Nαx-methyl amino acids, etc.) to convey special properties. Alternative synthetic amino acids that can be used include ornithine for lysine, fluorophenylalanine for phenylalanine, and norleucine for leucine or isoleucine. Other synthetic amino acids include 2-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, sarcosine, N-methylisoleucine, 6-N-methyllysine, and N-methylvaline. Additionally, by assigning specific amino acids at specific coupling steps, α-helices, β turns, β sheets, γ-turns, and cyclic peptides can be generated.

In a further embodiment, subunits of peptides that confer useful chemical and structural properties will be chosen. For example, peptides comprising D-amino acids will be resistant to L-amino acid-specific proteases in vivo. In addition, the present invention envisions preparing peptides that have more well defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a molecule would be resistant to peptide bond hydrolysis, e.g., protease activity. Such peptides would provide ligands with unique function and activity, such as extended half-lives in vivo due to resistance to metabolic breakdown, or protease activity. Furthermore, it is well known that in certain systems constrained peptides show enhanced functional activity [Hruby, Life Sciences, 31:189-199 (1982); Hruby et al., Biochem J., 268:249-262 (1990)]; the present invention provides a method to produce a constrained peptide that incorporates random sequences at all other positions.

Constrained and cyclic peptides. A constrained, cyclic or rigidized peptide may be prepared synthetically, provided that in at least two positions in the sequence of the peptide an amino acid or amino acid analog is inserted that provides a chemical functional group capable of crosslinking to constrain, cyclise or rigidize the peptide after treatment to form the crosslink. Cyclization will be favored when a turn-inducing amino acid is incorporated. Examples of amino acids capable of crosslinking a peptide are cysteine to form disulfides, aspartic acid to form a lactone or a lactam, and a chelator such as γ-carboxyl-glutamic acid (Gla) (Bachem) to chelate a transition metal and form a cross-link. Protected γ-carboxyl glutamic acid may be prepared by modifying the synthesis described by Zee-Cheng and Olson [Biophys. Biochem. Res. Commun., 94:1128-1132 (1980)]. A peptide in which the peptide sequence comprises at least two amino acids capable of crosslinking may be treated, e.g., by oxidation of cysteine residues to form a disulfide or addition of a metal ion to form a chelate, so as to crosslink the peptide and form a constrained, cyclic or rigidized peptide.

The present invention provides strategies to systematically prepare cross-links. For example, if four cysteine residues are incorporated in the peptide sequence, different protecting groups may be used (Hiskey, in The Peptides: Analysis, Synthesis, Biology, Vol. 3, Gross and Meienhofer, eds., Academic Press: New York, pp. 137-167 (1981); Ponsanti et al., Tetrahedron, 46:8255-8266 (1990)]. The first pair of cysteines may be deprotected and oxidized, then the second set may be deprotected and oxidized. In this way a defined set of disulfide cross-links may be formed. Alternatively, a pair of cysteines and a pair of chelating amino acid analogs may be incorporated so that the cross-links are of a different chemical nature.

Non-classical amino acids that induce conformational constraints. The following non-classical amino acids may be incorporated in the peptide in order to introduce particular conformational motifs: 1,2,3,4-tetrahydroisoquinoline-3-carboxylate [Kazmierski et al., J. Am. Chem. Soc., 113:2275-2283 (1991)]; (2S,3S)-methyl-phenylalanine, (2S,3R)-methyl-phenylalanine, (2R,3S)-methyl-phenylalanine and (2R,3R)-methyl-phenylalanine (Kazmierski and Hruby, Tetrahedron Lett. (1991)]; 2-aminotetrahydronaphthalene-2-carboxylic acid [Landis, Ph.D. Thesis, University of Arizona (1989)]; hydroxy-1,2,3,4-tetrahydroisoquinoline-3-carboxylate [Miyake et al., J. Takeda Res. Labs., 43:53-76 (1989)]; β-carboline (D and L) [Kazmierski, Ph.D. Thesis, University of Arizona (1988)]; HIC (histidine isoquinoline carboxylic acid) [Zechel et al., Int. J. Pep. Protein Res., 43 (1991)]; and HIC (histidine cyclic urea) (Dharanipragada).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog [Kemp et al., J. Org. Chem., 50:5834-5838 (1985)]; β-sheet inducing analogs [Kemp et al., Tetrahedron Lett., 29:5081-5082 (1988); β-turn inducing analogs [Kemp et al., Tetrahedron Lett., 29:5057-5060 (1988)]; ∝-helix inducing analogs (Kemp et al., Tetrahedron Lett., 29:4935-4938 (1988)]; γ-turn inducing analogs [Kemp et al., J. Org. Chem., 54:109:115 (1989)]; and analogs provided by the following references: Nagai and Sato, Tetrahedron Lett., 26:647-650 (1985); DiMaio et al., J. Chem. Soc. Perkin Trans., p. 1687 (1989); also a Gly-Ala turn analog [Kahn et al., Tetrahedron Lett., 30:2317 (1989)]; amide bond isostere [Jones et al., Tetrahedron Lett., 29:3853-3856 (1988)]; tretrazol [Zabrocki et al., J. Am. Chem. Soc., 110:5875-5880 (1988)]; DTC [Samanen et al., Int. J. Protein Pep. Res., 35:501:509 (1990)]; and analogs taught in Olson et al., J. Am. Chem. Sci., 112:323-333 (1990) and Garvey et al., J. Org. Chem., 56:436 (1990). Conformationally restricted mimetics of beta turns and beta bulges, and peptides containing them, are described in U.S. Pat. No. 5,440,013, issued Aug. 8, 1995 to Kahn.

Structure-Based Mutation Analysis

Protein structural analysis using NMR spectroscopy has several unique advantages. In addition to high-resolution three-dimensional structural information, the chemical shift assignments for the protein obtained in the structural study further provides a map of the entire protein at the atomic level, which can be used for structure-based biochemical analysis of protein-protein interactions. For example, the information generated from the NMR structural analysis can also serve to identify specific amino acid residues in the peptide-binding site for complementary mutagenesis studies. Specific focus can be placed on those residues that display long-range NOEs (particularly the side-chain NOEs in the ¹³C-NOESY data) between the bromoomain and a peptide comprising an acetyl-lysine.

To ensure mutant proteins are valid for functional analysis, it can be determined as to whether a mutation results in any significant perturbation of the overall conformation of the bromodomain, particularly the effects of mutation on the acetyl-lysine binding sites. NMR spectroscopy is a powerful method for examining the effects of such a mutation on the conformation of the protein. One can readily obtain information about the global conformation of a mutant protein from the proton (¹H) 1D spectrum, by examining the chemical shift dispersion and peak line-width of NMR signals of amide, aromatic and aliphatic protons. Moreover, 2D ¹H-¹⁵N HSQC spectra reveal details of the effects of a mutation on both local and global conformation of the protein, since every single ¹H/¹⁵N signal (both the chemical shift and line-shape) in the NMR spectrum is a “reporter” for a particular amino acid residue. Thus, to assess how mutations effect protein stability and the overall protein conformation, the ¹⁵N HSQC spectra of mutated proteins can be compared to that of the wild-type protein bromodomain.

Chemical-shift perturbations due to ligand binding have proven to be a reliable and sensitive probe for the ligand binding site of the protein. This is because the chemical-shift changes of the backbone amide groups are likely to reflect any changes in protein conformation and/or hydrogen bonding due to the peptide/ligand binding. To examine the effects of a mutation on the ligand binding (in this case the ligand is a peptide comprising an acetyl-lysine), peptide titration experiments can be conducted by following the changes of ¹H/¹⁵N signals of the mutant proteins as a function of the peptide concentration. These experiments indicate whether the acetyl-lysine binding site remains the same or changes in the mutants relative to the wild type protein. The effects of the mutation on the peptide binding affinity can also be examined by NMR spectroscopy. If the mutated proteins result in the reduction of the binding affinity, a change of the exchange phenomenon between the free and the ligand-bound signals should be observed in NMR spectrum. If the reduction in binding affinity causes the peptide binding to change from a slow exchange rate to a fast exchange rate, on the NMR time scale, then the peptide binding affinity can be determined from the NMR titration experiment. From these mutation analyses key amino acid residues that are important for binding a peptide comprising the acetyl-lysine can be identified. Such analysis has been exemplified below.

Protein Structure Determination by NMR Spectroscopy

The NMR results from the present invention are summarized by the atomic structure coordinates of the free form of the P/CAF bromodomain (Table 5), of the P/CAF bromodomain-acetyl-histamine complex (Table 6), and the Tat-P/CAF complex (Table 10). The NMR chemical shift assignments of the P/CAF bromodomain are included in the chemical shift table (Table 1) for the ¹H-¹⁵N HSQC spectrum of P/CAF bromodomain. The unambiguous NOE-derived Inter-proton Distance Restraints for the P/CAF bromodomain are in Table 2, the ambiguous NOE-derived Inter-proton Distance Restraints are in Table 3, and the ¹H bonding restraints are disclosed in Table 4. The NMR chemical shift assignments of the Tat-P/CAF complex are included in the chemical shift table (Table 11) for the ¹H-¹⁵N HSQC spectrum of Tat-P/CAF complex The unambiguous NOE-derived Inter-proton Distance Restraints for the Tat-P/CAF complex are in Table 13, the ambiguous NOE-derived Inter-proton Distance Restraints are in Table 14, and the ¹H bonding restraints are disclosed in Table 12.

Backbone and Side-chain Assignments: Sequence-specific backbone assignment can be achieved by using a suite of deuterium-decoupled triple-resonance 3D NMR experiments which include HNCA, HN(CO)CA, HN(CA)CB, HN(COCA)CB, HNCO, and HN(CA)CO experiments [Yamazaki, et al., J. Am. Chem. Soc. 116:11655-11666 (1994)]. The water flip-back scheme is used in these NMR pulse programs to minimize amide signal attenuation from water exchange. Sequential side-chain assignments are typically accomplished from a series of 3D NMR experiments with alternative approaches to confirm the assignments. These experiments include 3D ¹⁵N TOCSY—HSQC, HCCH-TOCSY, (H)C(CO)NH-TOCSY, and H(C)(CO)NH-TOCSY [see Clore and Gronenborn Meth. Enzymol. 239:249-363 (1994); Sattler et al., Prog. in Nuclear Magnetic Resonance Spec. 4:93-158 (1999)].

Stereospecific Methyl Groups: Stereospecific assignments of methyl groups of Valine and Leucine residues can be obtained from an analysis of carbon signal multiplet splitting using a fractionally ¹³C-labeled protein sample, which can be readily prepared using M9 minimal medium containing 10% ¹³C-/90%¹²C-glucose mixture [see Neri, et al., Biochemistry 28:7510-7516 (1989)].

Dihedral Angle Restraints: Backbone dihedral angle (Φ) constraints can be generated from the ³J_{HNHα} coupling constants measured in a HNHA-J experiment [see Vuister, G. & Bax, A. J. Am. Chem. Soc. 115:7772-7777 (1993)]. Side-chain dihedral angles (_{χ}1) can be obtained from short mixing time ¹⁵N-edited 3D TOCSY—HSQC [see Clore, et al., J. Biomol. NMR 1:13-22 (1991)] and 3D HNHB experiments [see Matson et al., J. Biomol. NMR 3:239-244 (1993)], which can also provide stereospecific assignments of β methylene protons.

Hydrogen Bonds Restraints: Amide protons that are involved in hydrogen bonds can be identified from an analysis of amide exchange rates measured from a series of 2D ¹H/¹⁵N HSQC spectra recorded after adding ²H₂O to the protein sample.

NOE Distance Restraints: Distance restraints are obtained from analysis of ¹⁵N, and ¹³C-edited 3D NOESY data, which can be collected with different mixing times to minimize spin diffusion problems. The nuclear Overhauser effect (NOE)-derived restraints are categorized as strong (1.8-3 Å), medium (1.8-4 Å) or weak (1.8-5 Å) based on the observed NOE intensities. A recently developed procedure for the iterative automated NOE analysis by using ARIA [see Nilges et al., Prog. NMR Spectroscopy 32:107-139 (1998)] can be employed which integrates with X-PLOR [Brunger, X-PLOR Version 3.1: A system for X-Ray crystallography and NMR, Yale University Press, New Haven, Conn., (1993)] for structural calculations. To ensure the success of ARIA/X-PLOR-assisted NOE analysis and structure calculations, the ARIA assigned NOE peaks can be manually confirmed.

Intermolecular NOE Distance Restrains: For the structural determination of a protein/peptide complex, intermolecular NOE distance restraints can be obtained from a ¹³C-edited (F₁) and ¹⁵N, and ¹³C-filtered (F₃) 3D NOESY data set collected for a sample containing isotope-labeled protein and non-labeled peptide.

Structure Calculations and Refinements: Structures of the protein can be generated using a distance geometry/simulated annealing protocol with the X-PLOR program [see Nilges,et al., FEBS Lett. 229:317-324 (1988); Kuszewski, et al., J. Biolmol. NMR 2:33-56 (1992); Brünger, A. T. X-PLOR Version 3.1: A system for X-Ray crystallography and NMR (Yale University Press, New Haven, Conn., 1993)]. The structure calculations can employ inter-proton distance restraints obtained from ¹⁵N- and ¹³C-resolved NOESY spectra. The initial low-resolution structures can be used to facilitate NOE assignments, and help identify hydrogen bonding partners for slowly exchanging amide protons. The experimental restraints of dihedral angles and hydrogen bonds can be included in the distance restraints for structure refinements.

Protein-Structure Based Design of Agonists and Antagonists of the Bromodomain-Acetyl-Lysine Binding Complex

Once the three-dimensional structure of the Bromodomain and the Bromodomain-acetyl-lysine binding complex are determined, a potential drug or agent (antagonist or agonist) can be examined through the use of computer modeling using a docking program such as GRAM, DOCK, or AUTODOCK [Dunbrack et al., 1997, supra]. This procedure can include computer fitting of potential agents to the bromodomain, for example, to ascertain how well the shape and the chemical structure of the potential ligand will complement or interfere with the interaction between the bromodomain and the acetyl-lysine [Bugg et al., Scientific American, December:92-98 (1993); West et al., TIPS, 16:67-74 (1995)]. Computer programs can also be employed to estimate the attraction, repulsion, and steric hindrance of the agent to the dimer-dimer binding site, for example. Generally the tighter the fit (e.g., the lower the steric hindrance, and/or the greater the attractive force) the more potent the potential drug will be since these properties are consistent with a tighter binding constant. Furthermore, the more specificity in the design of a potential drug the more likely that the drug will not interfere with related proteins. This will minimize potential side-effects due to unwanted interactions with other proteins.

Initially a potential drug could be obtained by screening a random peptide library produced by recombinant bacteriophage for example, [Scott and Smith, Science, 249:386-390 (1990); Cwirla et al., Proc. Natl. Acad. Sci., 87:6378-6382 (1990); Devlin et al., Science, 249:404-406 (1990)] or a chemical library. In particular, based on the NMR structural analysis provided herein, compounds that comprise an “acetyl-amine-like” structure as depicted in FIG. 12 are particularly good candidates. Examples of such “acetyl-lysine analogs” are included in FIG. 13.

An agent selected in this manner could be then be systematically modified (if necessary) by computer modeling programs until one or more promising potential drugs are identified. Such analysis has been shown to be effective in the development of HIV protease inhibitors [Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)]. Such computer modeling allows the selection of a finite number of rational chemical modifications, as opposed to the countless number of essentially random chemical modifications that could be made, any one of which might lead to a useful drug. Each chemical modification requires additional chemical steps, which while being reasonable for the synthesis of a finite number of compounds, quickly becomes overwhelming if all possible modifications needed to be synthesized. Thus, through the use of the three-dimensional structural analysis disclosed herein and computer modeling, a large number of these compounds can be rapidly screened on the computer monitor screen, and a few likely candidates can be determined without the laborious synthesis of untold numbers of compounds.

Once a potential drug (agonist or antagonist) is identified it can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively the potential drug may be synthesized de novo. As mentioned above, the de novo synthesis of one or even a relatively small group of specific compounds is reasonable in the art of drug design.

The potential drug can then be tested in any standard binding assay (including in high throughput binding assays) for its ability to bind to the ZA loop of a bromodomain. Alternatively the potential drug can be tested for its ability to modulate the binding of a bromodomain to acetylated histamine, for example. When a suitable potential drug is identified, a second NMR structural analysis can optionally be performed on the binding complex formed between the bromodomain-acetyl-lysine binding complex, or the bromodomain alone and the potential drug. Computer programs that can be used to aid in solving such three-dimensional structures include QUANTA, CHARMM, INSIGHT, SYBYL, MACROMODE, and ICM, MOLMOL, RASMOL, AND GRASP [Kraulis, J. Appl Crystallogr. 24:946-950 (1991)].

Using the approach described herein and equipped with the structural analysis disclosed herein, the three-dimensional structures of other bromodomain-acetyl-lysine binding complexes can more readily be obtained and analyzed. Such analysis will, in turn, allow corresponding drug screening methodology to be performed using the three-dimensional structures of such related complexes. For all of the drug screening assays described herein further refinements to the structure of the drug will generally be necessary and can be made by the successive iterations of any and/or all of the steps provided by the particular drug screening assay, including further structural analysis by NMR, for example.

Phage libraries for Drug Screening: Phage libraries have been constructed which when infected into host E. coli produce random peptide sequences of approximately 10 to 15 amino acids [Parmley and Smith, Gene 73:305-318 (1988), Scott and Smith, Science 249:386-249 (1990)]. Specifically, the phage library can be mixed in low dilutions with permissive E. coli in low melting point LB agar which is then poured on top of LB agar plates. After incubating the plates at 37° C. for a period of time, small clear plaques in a lawn of E. coli will form which represents active phage growth and lysis of the E. coli. A representative of these phages can be absorbed to nylon filters by placing dry filters onto the agar plates. The filters can be marked for orientation, removed, and placed in washing solutions to block any remaining absorbent sites. The filters can then be placed in a solution containing, for example, a radioactive bromodomain. After a specified incubation period, the filters can be thoroughly washed and developed for autoradiography. Plaques containing the phage that bind to the radioactive bromodomain can then be identified. These phages can be further cloned and then retested for their ability to bind to the bromodomain as before. Once the phage has been purified, the binding sequence contained within the phage can be determined by standard DNA sequencing techniques. Once the DNA sequence is known, synthetic peptides can be generated which are encoded by these sequences. These peptides can be tested, for example, for their ability to modulate the affinity of the bromodomain for its binding partner (e.g., Tat or a fragment of Tat containing the acetyl-lysine corresponding to position 50 of SEQ ID NO:45).

The effective peptide(s) can be synthesized in large quantities for use in in vivo models and eventually in humans to treat certain tumors. It should be emphasized that synthetic peptide production is relatively non-labor intensive, easily manufactured, quality controlled and thus, large quantities of the desired product can be produced quite cheaply. Similar combinations of mass produced synthetic peptides have been used with great success [Patarroyo, Vaccine, 10:175-178 (1990)].

Drug Screening Assays

The drug screening assays of the present invention may use any of a number of means for determining the interaction between an agent/drug (e.g., an acetyl-lysine analog) and a peptide comprising an acetyl-lysine and/or a bromodomain. Thus, standard high throughput drug screening procedures can be employed using a library of low molecular weight compounds, for example that can be screened to identify a binding partner for the bromodoamin. Any such chemical library can be used including those discussed above.

In a particular assay, a bromodomain (e.g., from P/CAF) is placed on or coated onto a solid support. Methods for placing the peptides or proteins on the solid support are well known in the art and include such things as linking biotin to the protein and linking avidin to the solid support. An agent is allowed to equilibrate with the bromodomain to test for binding. Generally, the solid support is washed and agents that are retained are selected as potential drugs. Alternatively, a peptide comprising an acetyl-lysine is placed on or coated onto a solid support. In a particular embodiment of this type, the peptide comprises the amino acid sequence of SEQ ID NO:4. In a preferred embodiment, the peptide comprises the amino acid sequence of SEQ ID NO: 46.

The agent may be labeled. For example, in one embodiment radiolabeled agents are used to measure the binding of the agent. In another embodiment the agents have fluorescent markers. In yet another embodiment, a Biocore chip (Pharmacia) coated with the bromodomain is used, for example and the change in surface conductivity can be measured.

In addition, since a number of proteins have been identified that contain bromodomains, and the binding partners of many of these proteins are known, the fact that the bromodomain specifically binds to an acetylated lysine as disclosed herein allows the identification and preparation of a number of potential modulators of the bromodomain-acetyl-lysine binding complex based on the amino acid sequences of the binding partners to the proteins. Such potential modulators include: ISYGR-AcK-KRRQRR (SEQ ID NO:4), ARKSTGG-AcK-APRKQL (SEQ ID NO:5) and QSTSRHK-ACK-LMFKTE (SEQ ID NO:6) which bind to the P/CAF bromodomain as shown in the Example, below. Such peptides also can be used, for example, as a starting point for the design of an inhibitor of the bromodomain-acetyl-lysine binding complex.

Alternatively, a drug can be specifically designed to bind to the ZA loop of a bromodomain for example, such as the P/CAF bromodomain, and be assayed through NMR based methodology [Shuker et al., Science 274:1531-1534 (1996) hereby incorporated by reference in its entirety.] In a particular embodiment, analogs of the binding partner of the bromodomain can be used in this analysis. One such peptide has the amino acid sequence of SEQ ID NO:4. In another embodiment of this type, the peptide has the amino acid sequence of SEQ ID NO:5. In another such embodiment of this type, the peptide has the amino acid sequence of SEQ ID NO:6. The assay begins with contacting a compound with a ¹⁵N-labeled bromodomain. Binding of the compound with the ZA loop of the bromodomain can be determined by monitoring the ¹⁵N- or ¹H-amide chemical shift changes in two dimensional ¹⁵N-heteronuclear single-quantum correlation (¹⁵N—HSQC) spectra upon the addition of the compound to the ¹⁵N-labeled bromodomain. Since these spectra can be rapidly obtained, it is feasible to screen a large number of compounds [Shuker et al., Science 274:1531-1534 (1996)]. A compound is identified as a potential ligand if it binds to the ZA loop of the bromodomain. In a further embodiment, the potential ligand can then be used as a model structure, and analogs to the compound can be obtained (e.g., from the vast chemical libraries commercially available, or alternatively through de novo synthesis). The analogs are then screened for their ability to bind the ZA loop of the bromodomain thus to obtain a ligand. An analog of the potential ligand is chosen as a ligand when it binds to the ZA loop of the bromodomain with a higher binding affinity than the potential ligand. In a preferred embodiment of this type the analogs are screened by monitoring the ¹⁵N- or ¹H-amide chemical shift changes in two dimensional ¹⁵N-heteronuclear single-quantum correlation (¹⁵N—HSQC) spectra upon the addition of the analog to the ¹⁵N-labeled bromodomain as described above.

In another further embodiment, compounds are screened for binding to two nearby sites on the bromodomain. In this case, a compound that binds a first site of the bromodomain does not bind a second nearby site. Binding to the second site can be determined by monitoring changes in a different set of amide chemical shifts in either the original screen or a second screen conducted in the presence of a ligand (or potential ligand) for the first site. From an analysis of the chemical shift changes the approximate location of a potential ligand for the second site is identified. Optimization of the second ligand for binding to the site is then carried out by screening structurally related compounds (e.g., analogs as described above). When ligands for the first site and the second site are identified, their location and orientation in the ternary complex can be determined experimentally either by NMR spectroscopy or X-ray crystallography. On the basis of this structural information, a linked compound is synthesized in which the ligand for the first site and the ligand for the second site are linked. In a preferred embodiment of this type the two ligands are covalently linked. This linked compound is tested to determine if it has a higher binding affinity for the bromodomain than either of the two individual ligands. A linked compound is selected as a ligand when it has a higher binding affinity for the bromodomain than either of the two ligands. In a preferred embodiment the affinity of the linked compound with the bromodomain is determined monitoring the ¹⁵N- or ¹H-amide chemical shift changes in two dimensional ¹⁵N-heteronuclear single-quantum correlation (¹⁵N—HSQC) spectra upon the addition of the linked compound to the ¹⁵N-labeled bromodomain as described above. A larger linked compound can be constructed in an analogous manner, e.g., linking three ligands which bind to three nearby sites on the bromodomain to form a multilinked compound that has an even higher affinity for the bromodomain than the linked compound.

Identification of New Bromodomains

By disclosing that protein bound acetyl-lysine is a binding partner for bromodomains, the present invention provides a method of identifying novel proteins that contain bromodomains. In short, a protein fragment or analog thereof comprising an acetyl-lysine or an acetyl-lysine analog can be used as bait to identify a binding partner that comprises a bromodomain. Any one of a number of procedures can be carried out to identify such a binding partner. One such assay comprises passing a cell extract over the bait peptide which is attached to a solid support. After washing the solid support to remove any non-specific binders, the bromodomain containing protein can be eluted from the solid support with an appropriate eluant. In a particular embodiment, the free bait peptide can be used in the elution. Other methodology includes the use of a yeast two-hybrid system, a GST pull down assay, ELISA, immunometric assays, and a modification of the CORT procedure of Schlessinger et al., (U.S. Pat. No. 5,858,686, hereby incorporated by reference in its entirety) for use with the bromodomain-acetyl-lysine binding complex.

Labels

Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the test and control marker gene. In the instance where a radioactive label, such as the isotopes ³H, ¹⁴C, ³²P, ³⁵S, ³⁶Cl, ⁵Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932 and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419-439 (1980) and in U.S. Pat. No. 4,857,453. Suitable enzymes include, but are not limited to, alkaline phosphatase, β-galactosidase, green fluorescent protein and its derivatives, luciferase, and horseradish peroxidase. Other labels for use in the invention include magnetic beads or magnetic resonance imaging labels.

Three-Dimensional Representation of the Structure of the Bromodomains

In addition, the present invention provides a computer that comprises a representation of a bromodomain (or a bromodomain-ligand complex, e.g., the Tat-P/CAF complex) in computer memory that can be used to screen for compounds that will or are likely to inhibit the bromodomain-ligand interaction. In a particular embodiment of the present invention the bromodomain-ligand complex is the Tat-P/CAF complex and the compound identified by the screen can used to prevent, retard the progression, treat and/or cure AIDS.

In a related embodiment, the computer can be used in the design of altered bromodomains that have either enhanced, or alternatively diminished binding activity activity. Preferably, the computer comprises portions of and/or all of the information contained in Tables 1-6 and 10-14. In a particular embodiment, the computer comprises: (i) a machine-readable data storage material encoded with machine-readable data, (ii) a working memory for storing instructions for processing the machine readable data, (iii) a central processing unit coupled to the working memory and the machine-readable data storage material for processing the machine-readable data into a three-dimensional representation, and (iv) a display coupled to the central processing unit for displaying the three-dimensional representation.

Thus the machine-readable data storage medium comprises a data storage material encoded with machine readable data which can comprise portions and/or all of the structural information contained in Tables 1-6 and 10-14. One embodiment for manipulating and displaying the structural data provided by the present invention is schematically depicted in FIG. 11. As depicted, the System 1, includes a computer 2 comprising a central processing unit (“CPU”) 3, a working memory 4 which may be random-access memory or “core” memory, mass storage memory 5 (e.g., one or more disk or CD-ROM drives), a display terminal 6 (e.g., a cathode-ray tube), one or more keyboards 7, one or more input lines 10, and one or more output lines 20, all of which are interconnected by a conventional bidirectional system bus 30. Input hardware 12, coupled to the computer 2 by input lines 10, may be implemented in a variety of ways. Machine-readable data may be inputted via the use of one or more modems 14 connected by a telephone line or dedicated data line 16. Alternatively or additionally, the input hardware 12 may comprise CD-ROM or disk drives 5. In conjunction with the display terminal 6, the keyboard 7 may also be used as an input device. Output hardware 22, coupled to computer 2 by output lines 20, may similarly be implemented by conventional devices. Output hardware 22 may include a display terminal 6 for displaying the three dimensional data. Output hardware might also include a printer 24, so that a hard copy output may be produced, or a disk drive 5, to store system output for later use, see also U.S. Pat. No.: 5,978,740, Issued Nov. 2, 1999, the contents of which are hereby incorporated by reference in their entireties. In operation, the CPU 3 (i) coordinates the use of the various input and output devices 12 and 22; (ii) coordinates data accesses from mass storage 5 and accesses to and from working memory 4; and (iii) determines the sequence of data processing steps. Any of a number of programs may be used to process the machine-readable data of this invention.

Antibodies to Portions of the Bromodomain that Interact with Acetyl-Lysine

According to the present invention, the bromodomains, and more particularly the ZA loops of the bromodomains and fragments thereof can be produced by a recombinant source, or through chemical synthesis, or through the modification of these peptides and fragments; and derivatives or analogs thereof, including fusion proteins, may be used as an immunogen to generate antibodies that specifically interfere with the formation of the bromodomain-acetyl-lysine binding complex. Similarly, antibodies can be raised against peptides that comprise one or more acetyl-lysine residues which also interfere with the formation of the bromodomain-acetyl-lysine binding complex. Such antibodies include but are not limited to polyclonal, monoclonal, chimeric, single chain, Fab fragments, and a Fab expression library. Various procedures known in the art may be used for the production of the polyclonal antibodies. For the production of antibody, various host animals can be immunized by injection with the peptide having the amino acid sequence of SEQ ID NO:3, for example, or a derivative (e.g., or fusion protein) thereof, including but not limited to rabbits, mice, rats, sheep, goats, etc. In one embodiment, the peptide can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the peptides or protein fragments of the present invention, or analog, or derivative thereof, any technique that provides for the production of antibody molecules by continuous cell lines in culture may be used. These include but are not limited to the hybridoma technique originally developed by Kohler and Milstein [Nature, 256:495-497 (1975)], as well as the trioma technique, the human B-cell hybridoma technique [Kozbor et al., Immunology Today, 4:72 (1983); Cote et al., Proc. Natl. Acad. Sci. U.S.A., 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. In an additional embodiment of the invention, monoclonal antibodies can be produced in germ-free animals utilizing technology described in PCT/US90/02545. In fact, according to the invention, techniques developed for the production of “chimeric antibodies” [Morrison et al., J. Bacteriol., 159:870 (1984); Neuberger et al., Nature, 312:604-608 (1984); Takeda et al., Nature, 314:452-454 (1985)] by splicing the genes from a mouse antibody molecule specific for the peptide having the amino acid sequence of SEQ ID NO:3, for example, together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies [U.S. Pat. Nos. 5,476,786 and 5,132,405 to Huston; U.S. Pat. No. 4,946,778] can be adapted to produce specific single chain antibodies. An additional embodiment of the invention utilizes the techniques described for the construction of Fab expression libraries [Huse et al., Science, 246:1275-1281 (1989)] to allow rapid and easy identification of monoclonal Fab fragments with the desired specificity. Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a ZA loop of a bromodomain, for example, one may assay generated hybridomas for a product which binds to a bromodomain fragment containing such an epitope and choose those which do not cross-react with bromodomain fragments that do not include that epitope. In a specific embodiment, antibodies that interfere with the formation of the bromodomain-acetyl-lysine complex can be generated. Such antibodies can be tested using the assays described and could potentially be used in anti-cancer therapies.

Administration

According to the invention, the component or components of a therapeutic composition, e.g., an agent of the invention that interferes with the bromodomain-acetyl-lysine binding complex such as the peptide having the amino acid sequence of SEQ ID NOs: 4, 5, 6, 46, or 47, or an acetyl-lysine analog as defined by FIG. 12 and exemplified in FIG. 13, and a pharmaceutically acceptable carrier, may be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration. In a preferred aspect, the agent of the present invention can cross cellular and nuclear membranes, which would allow for intravenous or oral administration. Strategies are available for such crossing, including but not limited to, increasing the hydrophobic nature of a molecule; introducing the molecule as a conjugate to a carrier, such as a ligand to a specific receptor, targeted to a receptor; and the like.

The present invention also provides for conjugating targeting molecules to such an agent. “Targeting molecule” as used herein shall mean a molecule which, when administered in vivo, localizes to desired location(s). In various embodiments, the targeting molecule can be a peptide or protein, antibody, lectin, carbohydrate, or steroid. In one embodiment, the targeting molecule is a peptide ligand of a receptor on the target cell. In a specific embodiment, the targeting molecule is an antibody. Preferably, the targeting molecule is a monoclonal antibody. In one embodiment, to facilitate crosslinking the antibody can be reduced to two heavy and light chain heterodimers, or the F(ab′)₂ fragment can be reduced, and crosslinked to the agent via the reduced sulfhydryl. Antibodies for use as targeting molecule are specific for a cell surface antigen.

In another embodiment, the therapeutic compound can be delivered in a vesicle, in particular a liposome [see Langer, Science, 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.]. In yet another embodiment, the therapeutic compound can be delivered in a controlled release system. For example, the agent may be administered using intravenous infusion, an implantable osmotic pump, a transdermal patch, liposomes, or other modes of administration. In one embodiment, a pump may be used [see Langer, supra; Sefton, CRC Crit. Ref. Biomed. Eng., 14:201 (1987); Buchwald et al., Surgery, 88:507 (1980); Saudek et al., N. Engl. J. Med., 321:574 (1989)]. In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem., 23:61 (1983); see also Levy et al., Science, 228:190 (1985); During et al., Ann. Neurol., 25:351 (1989); Howard et al., J. Neurosurg., 71:105 (1989)]. In yet another embodiment, a controlled release system can be placed in proximity of the therapeutic target, i.e., the bone marrow, thus requiring only a fraction of the systemic dose [see, e.g., Goodson, in Medical Applications of Controlled Release, supra, vol. 2, pp. 115-138 (1984)]. Other controlled release systems are discussed in the review by Langer [Science, 249:1527-1533 (1990)].

Pharmaceutical Compositions. In yet another aspect of the present invention, provided are pharmaceutical compositions of the above. Such pharmaceutical compositions may be for administration for injection, or for oral, pulmonary, nasal or other forms of administration. In general, comprehended by the invention are pharmaceutical compositions comprising effective amounts of a low molecular weight component or components, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Remington's Pharmaceutical Sciences, 18th Ed. [1990, Mack Publishing Co., Easton, Pa. 18042] pages 1435-1712 which are herein incorporated by reference. The compositions may be prepared in liquid form, or may be in dried powder, such as lyophilized form.

Oral Delivery. Contemplated for use herein are oral solid dosage forms, which are described generally in Remington's Pharmaceutical Sciences, 18th Ed.1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include an agent of the present invention (or chemically modified forms thereof) and inert ingredients which allow for protection against the stomach environment, and release of the biologically active material in the intestine. Also specifically contemplated are oral dosage forms of the above derivatized component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. An example of such a moiety is polyethylene glycol. For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine. The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression. One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell. Disintegrants may be included in the formulation of the therapeutic into a solid dosage form. Materials used as disintegrates include but are not limited to starch, including the commercial disintegrant based on starch, Explotab. Binders also may be used to hold the therapeutic agent together to form a hard tablet and include materials from natural products such as acacia, tragacanth, starch and gelatin. An anti-frictional agent may be included in the formulation of the therapeutic to prevent sticking during the formulation process. Lubricants may be used as a layer between the therapeutic and the die wall. Glidants that might improve the flow properties of the drug during formulation and to aid rearrangement during compression also might be added. The glidants may include starch, talc, pyrogenic silica and hydrated silicoaluminate. In addition, to aid dissolution of the therapeutic into the aqueous environment a surfactant might be added as a wetting agent. Additives which potentially enhance uptake of the protein (or derivative) are for instance the fatty acids oleic acid, linoleic acid and linolenic acid.

Nasal Delivery. Nasal delivery of an agent of the present invention (or derivative) is also contemplated. Nasal delivery allows the passage of a peptide, for example, to the blood stream directly after administering the therapeutic product to the nose, without the necessity for deposition of the product in the lung. Formulations for nasal delivery include those with dextran or cyclodextran.

Transdermal administration. Various and numerous methods are known in the art for transdermal administration of a drug, e.g., via a transdermal patch. Transdermal patches are described in for example, U.S. Pat. No. 5,407,713, issued Apr. 18, 1995 to Rolando et al.; U.S. Pat. No. 5,352,456, issued Oct. 4, 1004 to Fallon et al.; U.S. Pat. No. 5,332,213 issued Aug. 9, 1994 to D'Angelo et al.; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al.; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al.; and U.S. Pat. No. 4,921,475, issued May 1, 1990 to Sibalis, the disclosure of each of which is incorporated herein by reference in its entirety. It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

Pulmonary Delivery. Also contemplated herein is pulmonary delivery of the pharmaceutical compositions of the present invention. A pharmaceutical composition of the present invention is delivered to the lungs of a mammal while inhaling and traverses across the lung epithelial lining to the blood stream. Other reports of this include Adjei et al. [Pharmaceutical Research, 7:565-569 (1990); Adjei et al., International Journal of Pharmaceutics, 63:135-144 (1990) (leuprolide acetate); Braquet et al., Journal of Cardiovascular Pharmacology, 13(suppl. 5): 143-146 (1989) (endothelin-1); Hubbard et al., Annals of Internal Medicine, Vol. III, pp. 206-212 (1989) (α1-antitrypsin); Smith et al., J. Clin. Invest., 84:1145-1146 (1989) (α-1-proteinase); Oswein et al., “Aerosolization of Proteins”, Proceedings of Symposium on Respiratory Drug Delivery II, Keystone, Colo., March, (1990) (recombinant human growth hormone); Debs et al., J. Immunol., 140:3482-3488 (1988) (interferon-γ and tumor necrosis factor alpha); Platz et al., U.S. Pat. No. 5,284,656 (granulocyte colony stimulating factor)]. A method and composition for pulmonary delivery of drugs for systemic effect is described in U.S. Pat. No. 5,451,569, issued Sep. 19, 1995 to Wong et al.

A subject in whom administration of an agent of the present invention is an effective therapeutic regiment for cancer, for example, is preferably a human, but can be any animal. Thus, as can be readily appreciated by one of ordinary skill in the art, the methods and pharmaceutical compositions of the present invention are particularly suited to administration to any animal, e.g., for veterinary medical use, particularly for a mammal, and including, but by no means limited to, domestic animals, such as feline or canine subjects, farm animals, including bovine, equine, caprine, ovine, and porcine subjects, wild animals (whether in the wild or in a zoological garden), research animals, such as mice, rats, rabbits, goats, sheep, pigs, dogs, cats, avian species, such as chickens, turkeys, and songbirds.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric. All publications referred to herein are specifically incorporated by reference in their entirety.

### Example 1

Structure and Ligand of a Histone Acetyltransferase Bromodomain

Sample preparation: The bromodomain of P/CAF (residues 719-832 of SEQ ID NO: 2) was subcloned into the pET14b expression vector (Novagen) and expressed in Escherichia coli BL21(DE3) cells. Uniformly ¹⁵N- and ¹⁵N/¹³C-labelled proteins were prepared by growing bacteria in a minimal medium containing ¹⁵NH₄Cl with or without ¹³C₆-glucose. A uniformly ¹⁵N/¹³C-labelled and fractionally deuterated protein sample was prepared by growing the cells in 75% ²H₂O. The bromodomain was purified by affinity chromatography on a nickel-IDA column (Invitrogen) followed by the removal of poly-His tag by thrombin cleavage. The final purification of the protein was achieved by size-exclusion chromatography. The acetyl-lysine-containing peptides were prepared on a MilliGen 9050 peptide synthesizer (Perkin Elmer) using Fmoc/HBTU chemistry. Acetyl-lysine was incorporated using the reagent Fmoc-Ac-Lys with HBTU/DIPEA activation. NMR samples contained approximately 1 mM protein in 100 mM phosphate buffer of pH 6.5 and 5 mM perdeuterated DTT and 0.5 mM EDTA in H₂O/²H₂O (9/1) or ²H₂O.

NMR spectroscopy: All NMR spectra were acquired at 30° C. on a Bruker DRX600 or DRX500 spectrometer. The backbone assignments of the ¹H, ¹³C, and ¹⁵N resonances were achieved using deuterium-decoupled triple-resonance experiments of HNCACB and HN(CO)CACB [Yamazaki et al., J. Am. Chem. Soc. 116:11655-11666 (1994)] recorded using the uniformly ¹⁵N/¹³C-labeled and fractionally deuterated protein. The side-chain atoms were assigned from 3D HCCH-TOCSY [Clore and Gronenborn, Meth. Enzymol. 239:249-363 (1994)] and (H)C(CO)NH-TOCSY [Logan et al., J. Biolmol. NMR 3:225-231 (1993)] data collected on the uniformly ¹⁵N/¹³C-labeled protein. Stereospecific assignments of methyl groups of the Val and Leu residues were obtained using a fractionally ¹³C-labeled sample [Neri et al., Biochemistry 28:7510-7516 (1989)]. The NOE-derived distance restraints were obtained from ¹⁵N- or ¹³C-edited 3D NOESY spectra. φ-angle restraints were determined based on the ³J_{HN,Hα} coupling constants measured in a 3D HNHA spectrum [Clore and Gronenborn, Meth. Enzymol. 239:249-363 (1994)]. Slowly exchanging amide protons were identified from a series of 2D ¹⁵N—HSQC spectra recorded after the H₂O buffer was changed to a ²H₂O buffer. The intermolecular NOEs used in defining the structure of the bromodomain/Ac-histamine complex were detected in ¹³C-edited (F₁), ¹³C/¹⁵N-filtered (F₃) 3D NOESY spectrum [Clore and Gronenborn, Meth. Enzymol. 239:249-363 (1994)]. All NMR spectra were processed with the NMRPipe/NMRDraw programs and analyzed using NMRView [Johnson and Blevins, J. Biomol., NMR 4:603-614 (1994)].

Structure calculations: Structures of the bromodomain were calculated with a distance geometry/simulated annealing protocol using the X-PLOR program [Brunger, X-PLOR Version 3.1: A system for X-Ray crystallography and NMR, Yale University Press, New Haven, Conn., (1993)]. A total of 1324 manually assigned NOE-derived distance restraints were obtained from the ¹⁵N- and ¹³C-edited NOE spectra. Further analysis of the NOE spectra was carried out by the iterative automated assignment procedure using ARIA [Nilges and O'Donoghue, Prog. NMR Spectroscopy 32:107-139 (1998)], which integrates with X-PLOR for structure calculations. A total of 1519 unambiguous and 590 ambiguous distance restraints were identified from the NOE data by ARIA, many of which were checked and confirmed manually. The ARIA-assigned distance restraints were in agreement with the structures calculated using only the manually assigned NOE distance restraints, 28 hydrogen-bond distance restraints for 14 hydrogen bonds, and 54φ-angle restraints. The final structure calculations employed a total of 3515 NMR experimental restraints obtained from the manual and the ARIA-assisted assignments, 2843 of which were unambiguously assigned NOE-derived distance restraints that comprise of 1077 intra-residue, 621 sequential, 550 medium-range, and 595 long-range NOEs. For the ensemble of the final 30 structures, no distance and torsional angle restraints were violated by more than 0.3 Å and 5°, respectively. The total, distance violation, and dihedral violation energies were 178.7±2.4 kcal mol⁻¹, 41.6±0.9 kcal mol⁻¹, and 0.50±0.06 kcal mol⁻¹, respectively. The Lennard-Jones potential which was not used during any refinement stage, was −526.2±16.8 kcal mol⁻¹ for the final structures. Ramachandran plot analysis of the final structures (residues 727-828) with Procheck-NMR [Laskowski et al., J. Biolmol. NMR 8:477-486 (1996)] showed that 71.0±0.6%, 23.8±0.6%, 3.5±0.2%, and 1.7±0.2% of the non-Gly and non-Pro residues were in the most favorable, additionally allowed, generously allowed, and disallowed regions, respectively. The corresponding values for the residues in the four α-helices (residues 727-743, 770-776, 785-802, and 807-827) were 88.9±0.4%, 11.0±0.4%, 0.1±0.1%, and 0.0±0.0%, respectively. The structure of the bromodomain/acetyl-histamine complex was determined using the free form structure and additional 25 intermolecular and 5 intra-ligand NOE-derived distance restraints.

Site-directed mutagenesis: Mutant proteins were prepared using the QuickChange site-directed mutagenesis kit (Stratagene). The presence of appropriate mutations was confirmed by DNA sequencing.

Ligand titration: Ligand titration experiments were performed by recording a series of 2D ¹⁵N- and ¹³C—HSQC spectra on the uniformly ¹⁵N-, and ¹⁵N/¹³C-labelled bromodomain (˜0.3 mM), respectively, in the presence of different amounts of ligand concentration ranging from 0 to approximately 2.0 mM. The protein sample and the stock solutions of the ligands were all prepared in the same aqueous buffer containing 100 mM phosphate and 5 mM perdeuterated DTT at pH 6.5.

The full length nucleic acid sequence of the human p300 /CBP-associated factor (P/CAF) was obtained from GenBank. Accession No: U57317.2 (SEQ ID NO:1). The full length protein sequence of the human p300/CBP-associated factor (P/CAF) was obtained from GenBank. Accession No: U57317.2, (SEQ ID NO:2).

Results. The P/CAF bromodomain represents an extensive family of bromodomains (FIG. 1). A large number of long-range nuclear Overhauser enhancement (NOE)-derived distance restraints were identified in the NMR data of the P/CAF bromodomain, yielding a well-defined three-dimensional structure (FIGS. 2A-2D). Table 1 shows the NMR chemical shift assignment of the P/CAF bromodomain. Table 2 shows the Unambiguous NOE-derived distance restraints. Table 3 shows the Ambiguous NOE-derived distance restraints. Table 4 shows the Hydrogen bond restraints. The NMR structure coordinates of the P/CAF bromodomain in the free and complexed to acetyl-histamine are shown in Tables 5 and 6, respectively. The structure consists of a four-helix bundle (helices α_{Z}, α_{A}, α_{B}, and α_{C}) with a left-handed twist, and a long intervening loop between helices α_{Z} and α_{A} (termed the ZA loop, FIG. 2E). The four amphipathic α-helices are packed tightly against one another in an antiparallel manner, with crossing angles for adjacent helices of ˜16-20°. The up-and-down four-helix bundle can adapt two topological folds with opposite handedness (FIGS. 2F-2G). The right-handed four-helix bundle fold occurs more commonly and is seen in proteins such as hemerythrin and cytochrome b₅₆₂. The left-handed fold of the bromodomain structure is less common, but also observed in proteins such as cytochrome b₅ and T4 lysozyme [Richardson, J., Adv.Protein Chem., 34:167-339 (1989); Presnell and Cohen, Proc. Natl. Acad. Sci. USA 86:6592-6596 (1989)]. This topological difference arises from the orientation of the loop between the first two helices (FIGS. 2F-2G). The right-handed four-helix bundle proteins have a relatively short hairpin-like connection between the first two helices, which makes the “preferred” turn to the right at the top of the first helix [Richardson, J., Adv.Protein Chem., 34:167-339 (1989); Presnell and Cohen, Proc. Natl. Acad. Sci. USA 86:6592-6596 (1989); Weber and Salemme, Nature 287:82-84 (1980)]. In contrast, proteins with the left-handed fold usually have a long loop after the first helix and often contain additional secondary structural elements at the base of the helix bundle [Richardson, J., Adv.Protein Chem., 34:167-339 (1989); Presnell and Cohen, Proc. Natl. Acad. Sci. USA 86:6592-6596 (1989)]. In the bromodomain structure, this long ZA loop has a defined conformation and is packed against the loop between helices α_{B} and α_{C} (termed the BC loop) to form a hydrophobic pocket. These tertiary interactions between the two loops appear to favor the left turn of the ZA loop, resulting in the left-handed four-helix bundle fold of the bromodomain. The hydrophobic pocket formed by loops ZA and BC is lined by residues Val752, Ala757, Tyr760, Val763, Tyr802 and Tyr809 (FIG. 2H), and appears to be a site for protein-protein interactions (see below). The pocket is located at one end of the four-helix bundle, opposite to the N- and C-termini of the protein. Interestingly, the ZA loop varies in length amongst different bromodomains, but almost always contains residues corresponding to Phe748, Pro751, Pro758, Tyr760, and Pro767 (FIG. 1). The conservation of these residues within the ZA loop as well as residues within the α-helical regions implies a similar left-handed four-helix bundle structure for the large family of bromodomains.

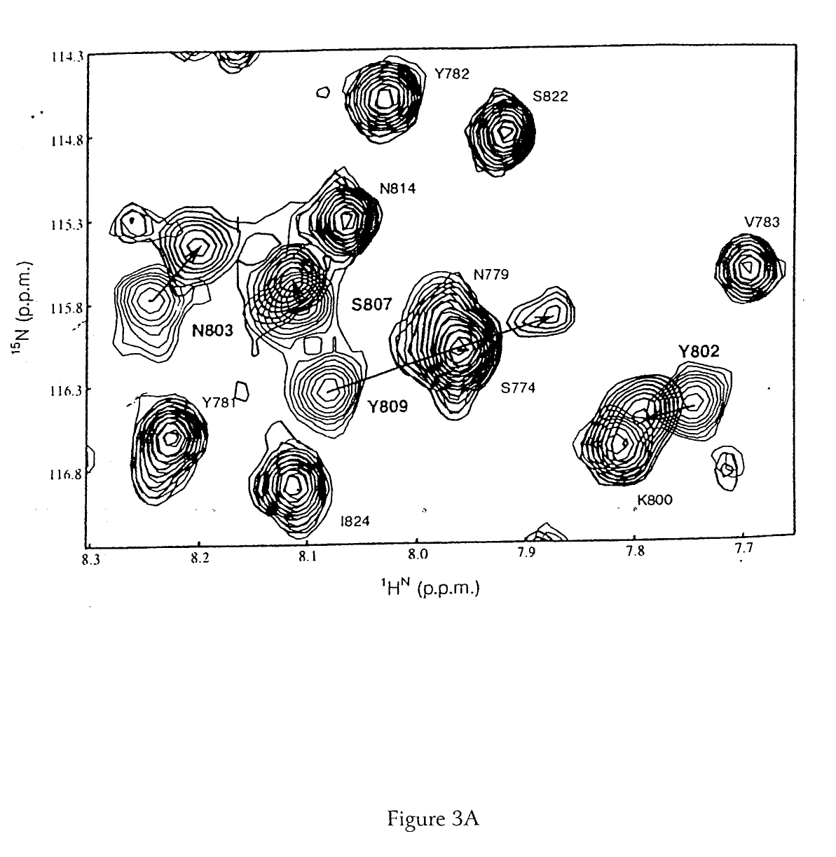

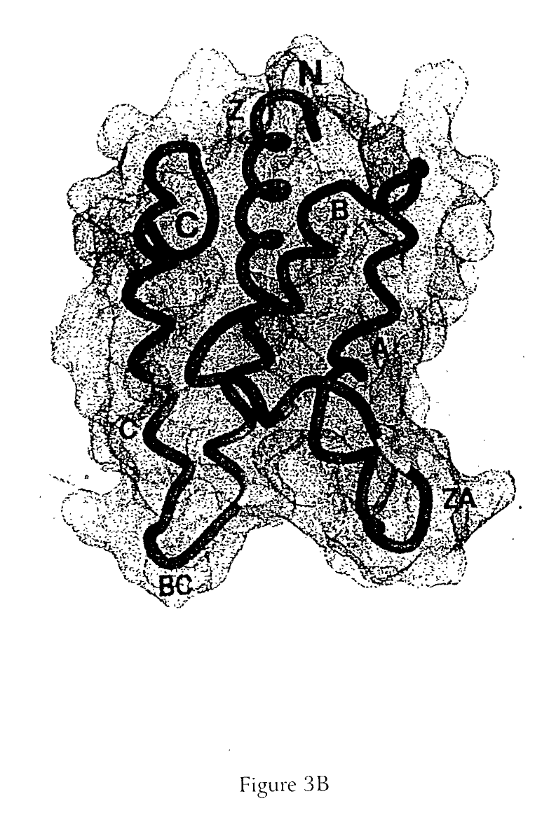

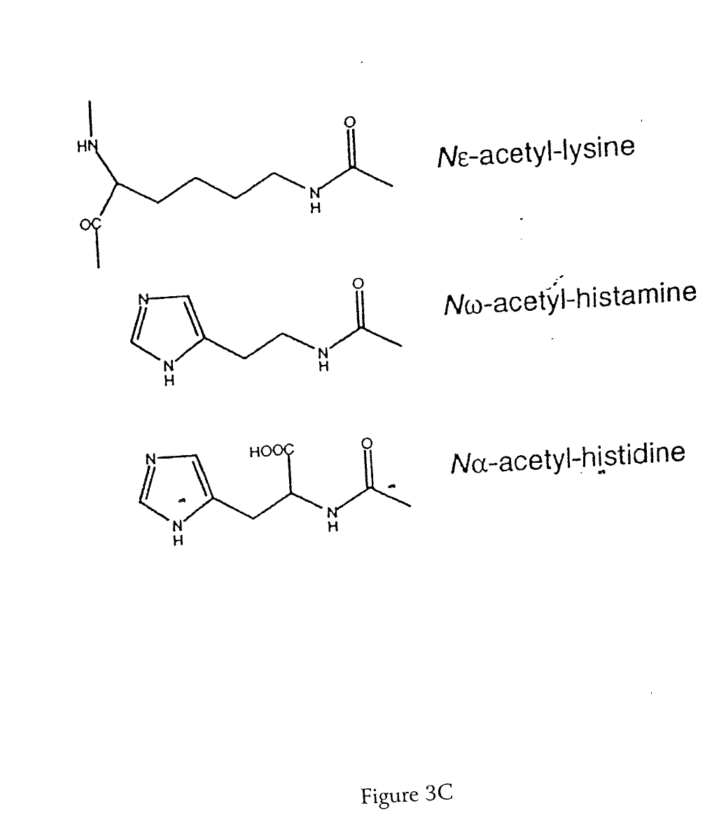

The modular bromodomain structure supports the idea that bromodomain can act as a functional unit for protein-protein interactions. The observation that bromodomains are found in nearly all known nuclear HATs (A-type) that are known to promote transcription-related acetylation of histones on specific lysine residues, but not present in cytoplasmic HATs (B-type), prompted the determination of whether bromodomains can interact with acetyl-lysine (AcK). The NMR titration of the P/CAF bromodomain were performed with a peptide (SGRGKGG-_{Ac}K-GLGK) derived from histone H4, in which Lys8 is acetylated (Lys8 is the major acetylation site in H4 for GCN5, a yeast homologue of P/CAF). Remarkably, the bromodomain could indeed bind the AcK peptide. Moreover, this interaction appeared to be specific, based on the ¹⁵N—HSQC spectra which showed that only a limited number of residues underwent chemical shift changes as a function of peptide concentration (FIG. 3A). Conversely, the NMR titration of the bromodomain with a non-acetylated, but otherwise identical H4 peptide, showed no noticeable chemical shift changes, demonstrating that the interaction between the bromodomain and the lysine-acetylated H4 peptide was dependent upon acetylation of lysine. The dissociation constant (K_{D}) for the AcK peptide was estimated to be 346±54 μM. This binding is likely reinforced through additional interactions between bromodomain-containing proteins and target proteins. Notably, many chromatin-associated proteins contain two or multiple bromodomains (FIG. 1). Indeed, binding with another lysine-acetylated peptide (RKSTGG-_{Ac}K-APRKQ) derived from the major acetylation site on histone H3 (residues 9-20) was also observed. Together, these data demonstrate that the P/CAF bromodomain has the ability to bind AcK peptides in an acetylation dependent manner.

Intriguingly, the bromodomain residues that exhibited the most significant ¹H and ¹⁵N chemical shift changes on peptide binding are located near the hydrophobic pocket between the ZA and BC loops (FIG. 3B). Because a similar pattern of amide chemical shift changes was observed with the two different AcK-containing peptides, it was surmised that the hydrophobic cavity is the primary binding site for AcK. This hypothesis was further supported by titration with acetyl-histamine, which mimics the chemical structure of the AcK side-chain (FIG. 3C). Both ¹⁵N- and ¹³C—HSQC spectra showed that interaction with acetyl-histamine was also acetylation-dependent, involving the same set of residues that showed chemical shift perturbations with similar concentration dependence. It should be noted that the bromodomain did not bind to the amino acids acetyl-lysine or acetyl-histidine alone, possibly due to the presence of the charged amino, carboxyl, or caboxylate group adjacent to the acetyl moiety (FIG. 3C). Taken together, these results strongly suggest that the P/CAF bromodomain can interact with acetyl-lysine-containing proteins in a specific manner, and that this interaction is localized to the bromodomain hydrophobic cavity.

To identify the key residues involved in bromodomain-AcK recognition, the NMR structure of the P/CAF bromodomain in complex with acetyl-histamine was elucidated. As anticipated, the acetylated moiety binds in the bromodomain hydrophobic pocket (FIG. 4). The intermolecular interactions are largely hydrophobic in nature, with the methyl group of acetyl-histamine making extensive contacts with the side-chains of Val752, Ala757, and Tyr760, and the methylene groups of acetyl-histamine displaying specific NOEs to Val752, Ala757, Tyr760, Tyr802, and Tyr809. No intermolecular NOEs were observed for the imidazole ring of acetyl-histamine. From the spectral analysis it is clear that the structure of the bromodomain is very similar in both the free and complex forms. It is worth noting that the bromodomain-AcK recognition is reminiscent of the interactions between the histone acetyltransferase Hat1 and acetyl-CoA. Although the binding pockets of these two otherwise structurally unrelated proteins are composed of different secondary structural elements, the nature of acetyl-lysine recognition has striking similarities. In particular, Tyr809, Tyr802, Tyr760, and Val752 in the bromodomain appear to be related to Phe220, Phe261, Val254, and Ile217 of Hat1, respectively, in their interactions with the acetyl moiety. This observation may suggest an evolutionary convergent mechanism of acetyl-lysine recognition between bromodomains and histone acetyltransferases. To determine the relative contributions of residues within the hydrophobic cavity in bromodomain-AcK binding, site-directed mutagenesis was used to alter residues Tyr809, Tyr802, Tyr760, and Val752 (Table 7).

TABLE 7
Structural and Functional Analysis of the P/CAF Bromodomain Mutants

| Bromodomain Protein | Structural Integrity^{a} | H4 AcK-Peptide Binding |
|---|---|---|
| Wild-Type | ++++ | 346 ± 54 |
| Tyr809Ala | ++++ | No Binding^{c} |
| Tyr802Ala | +++ | >10,000^{d} |
| Tyr760Ala | +++ | >10,000 |
| Val752Ala | ++ | >10,000 |
| ^{a}The effects of mutations on the structural integrity of the bromodomain were assessed by using the ¹⁵N-HSQC spectra. The amide ¹H/¹⁵N resonances of the mutant proteins were compared to those of the wild-type bromodomain to determine if the particular mutations lead to global or local structure disruption. Severe line-broadening of the amide resonances would indicate protein conformational exchange due to a decrease of structure stability resulting from | | |
| # point mutations. Structural integrity of the mutant proteins is expressed here relative to that of the wild-type, using the signs of “++++” for as stable as the wild-type, “+++” for mildly destabilized, “++” for moderately destabilized, and “−” for completely unfolded. ^{b}The ligand binding affinity (K_{D}) of the bromodomain proteins was estimated by following chemical shift changes of amide peaks in the | | |
| # ¹⁵NHSQC spectra as a function of the ligand concentration. ^{c}No detectable ligand binding observed in the NMR titration. ^{d}Ligand binding affinity was significantly reduced and beyond the limit for reliable measurements by NMR titration. | | |

Substitution of Ala for Tyr809 completely abrogated the bromodomain binding to the lysine-acetylated H4 peptide, while the Tyr802Ala, Tyr760Ala, and Val752Ala mutants had significantly reduced ligand binding affinity. To assess whether these mutations disrupted the overall bromodomain fold, the ¹⁵N—HSQC spectra of the mutants was compared to that of the wild-type protein. For the Tyr809Ala mutant, the amide chemical shifts were only affected for a few residues near the mutation site. However, mutations of the other residues in the hydrophobic binding pocket perturbed the local protein conformation to greater extents, particularly the ZA loop (Table 7). Thus, the NMR structural analysis and the mutagenesis studies show that Tyr809, which is structurally supported by Trp746 and Asn803 (FIG. 4), is essential for the bromodomain interaction with the acetyl group of acetyl-lysine, while residues of Tyr802, Tyr760, and Val752 likely play both structural and functional roles in the recognition. These residues are highly conserved throughout the bromodomain family (FIG. 1), suggesting that recognition of acetyl-lysine may be a feature of bromodomains, in general. Therefore, Val752, Ala757, Tyr760, Tyr802, Asn803, and Tyr809 are key amino acid residues for the P/CAF bromodomain binding to acetyl-lysine.

TABLE 8
Amino Acid Sequences of Bromodomains Identified in FIG. 1

| PROTEIN | SEQ ID | GenBank | PROTEIN | SEQ ID | GenBank |
|---|---|---|---|---|---|
| hsp/CAF | 7 | U57317 | dmFSH-2 | 25 | |
| hsGCN5 | 8 | U57136 | scBDF1-2 | 26 | |
| ttP55 | 9 | U47321 | hsBR140 | 27 | JC2069 |
| scGCN5 | 10 | Q03330 | hsSMAP | 28 | X87613 |
| hsP300 | 11 | A54277 | ggPB1-1 | 29 | X90849 |
| hsCBP | 12 | S39162 | ggPB1-2 | 30 | |
| mmCBP | 13 | S39161 | ggPB1-3 | 31 | |
| ceYNJ1 | 14 | P34545 | ggPB1-4 | 32 | |
| hsCCG1-1 | 15 | P21675 | ggPB1-5 | 33 | |
| msCCG1-1 | 16 | D26114 | spBRO-1 | 34 | S54260 |
| hsCCG1-2 | 17 | | spBRO-2 | 35 | |
| msCCG1-2 | 18 | | hsSNF2a | 36 | S45251 |
| hsRing3-1 | 19 | P25440 | hsBRG1 | 37 | S39039 |
| hsORFX-1 | 20 | D26362 | ggBRM | 38 | X91638 |
| dmFSH-1 | 21 | P13709 | ggBRG1 | 39 | X91637 |
| scBDF1-1 | 22 | P35817 | hsTIF1b | 40 | X97548 |
| hsRing3-2 | 23 | | mmTIF1b | 41 | X99644 |
| hsORFX-2 | 24 | | mmTlF1a | 42 | S78219 |

### Example 2

Structural Insights into HIV-1 TAT Transactivation via P/CAF

Whereas the life cycle of HIV is still being elucidated, it is currently accepted that HIV binds to CD4 protein of a host T cell or macrophage and with the aid of a chemokine receptor (e.g., CCR5 or CXCR4) enters the host cell. Once in the host cell, the retrovirus, HIV-1, is converted to a DNA by reverse transcriptase and the expression of the HIV-1 genome is dependent on a complex series of events that are believed to be under the control of two viral regulatory proteins, Tat and Rev [Romano et al., J.CellBiochem. 75(3):357-368 (1999)]. Rev controls post-translational events, whereas, Tat (the trans-activator protein) functions to stimulate the production of full-length HIV transcripts and viral replication in infected cells. The Tat protein transactivates the transcription of HIV-1 starting at the 5′ long terminal repeat (LTR) [Romano et al., J.CellBiochem. 75(3):357-368 (1999)] by recruiting one or more carboxyl-terminal domain kinases to the HIV-1 promoter. More specifically, Tat stimulates transcription from the LTR at a hairpin element, the transactivation responsive region (TAR) [Kiernan et al., EMBO J. 18:6106-6118 (1999)] at least in part by interacting with and thereby recruiting the carboxyl-terminal domain kinase, i.e., the positive transcriptional elongation factor (P-TEFb) to the TAR RNA element [Garber et al., Mol.Cell.Biol. 20(18):6958-6969 (2000)]. P-TEFb is a muti-subunit kinase that minimally comprises a heterodimer consisting of the regulatory cyclin T1 and its corresponding catalytic subunit, cyclin-dependent kinase 9 (CDK9). P-TEFb acts by phosphorylating the carboxyl-terminal domain of RNA polymerase II [Peng et al., J.Biol.Chem. 274 (49):34527-34530 (1999); Romano et al., J.CellBiochem. 75(3):357-368 (1999)].

Recently, it has been shown that HIV-1 Tat transcription activity is regulated through lysine acetylation by, and association with the histone acetyltransferases (HATs) p300/CBP and the p300/CBP-associating factor (P/CAF), which specifically acetylate Lysine 50 (K50) and Lysine 28 (K28) of the Tat protein, respectively [Kiernan et al., EMBO J. 18:6106-6118 (1999); Ott et al., Curr. Biol. 9:1489-1492 (1999)]. Notably, the acetylation of K50 by the transcriptional co-activator p300/CBP is on the C-terminal arginine-rich motif (ARM) of Tat, which is essential for its binding to the TAR RNA element and for nuclear localization, [Kiernan et al., EMBO J. 18:6106-6118 (1999); Ott et al., Curr. Biol. 9:1489-1492 (1999)]. Acetylation of K28 of Tat by P/CAF enhances Tat binding to P-TEFb, whereas acetylation of K50 of Tat by P300/CBP promotes the dissociation of Tat from the TAR RNA element. This dissociation of Tat from the TAR RNA element occurs during early transcription elongation [Kiernan et al., EMBO J. 18:6106-6118 (1999)]. However, heretofore, little else was known regarding the relationship of these HATs with Tat after the acetylation has occurred.

Sample preparation: The bromodomain of P/CAF (residues 719-832) was subcloned into the pET14b expression vector (Novagen) and expressed in Escherichia coli BL21(DE3) cells. Uniformly ¹⁵N- and ¹⁵N/¹³C-labeled proteins were prepared by growing bacteria in a minimal medium containing ¹⁵NH₄Cl with or without ¹³C₆-glucose. A uniformly ¹⁵N/¹³C-labeled and fractionally deuterated protein sample was prepared by growing the cells in 75% ²H₂O. The bromodomain was purified by affinity chromatography on a nickel-IDA column (Invitrogen) followed by the removal of poly-His tag by thrombin cleavage. The final purification of the protein was achieved by size-exclusion chromatography. The acetyl-lysine-containing peptides were prepared on a MilliGen 9050 peptide synthesizer (Perkin Elmer) using Fmoc/BBTU chemistry. Acetyl-lysine was incorporated using the reagent Fmoc-Ac-Lys with HBTU/DIPEA activation. NMR samples contained ˜0.5 mM protein in complex with the lysine-acetylated Tat peptide in 100 mM phosphate buffer of pH 6.5 and 5 mM perdeuterated DTT and 0.5 mM EDTA in H₂O/²H₂O (9/1) or ²H₂O. The bromodomain-containing constructs from P/CAF, CBP and TIF-1β were cloned into pGEX4T-3 vector (Pharmacia). These recombinant GST-fusion proteins were expressed in BL21 (DE3) codon plus cell line, and purified by using glutathione sepharose column.

NMR spectroscopy: All NMR spectra were acquired at 30° C. on a Bruker DRX600 or DRX500 spectrometer. The backbone assignments of the ¹H, ¹³C, and ¹⁵N resonances were achieved using deuterium-decoupled triple-resonance experiments of HNCACB and HN(CO)CACB [Yamazaki et al., J. Am. Chem. Soc. 116:11655-11666 (1994)] recorded using the uniformly ¹⁵N/¹³C-labelled and fractionally deuterated protein. The side-chain atoms were assigned from 3D HCCH-TOCSY [Clore and Gronenborn, Meth. Enzymol. 239:249-363 (1994)] and (H)C(CO)NH-TOCSY [Logan et al., J. Biolmol. NMR 3:225-231 (1993)] data collected on the uniformly ¹⁵N/¹³C-labeled protein. Stereospecific assignments of methyl groups of the valine and leucine residues were obtained using a fractionally ¹³C-labeled sample [Neri et al., Biochemistry 28:7510-7516 (1989)]. The NOE-derived distance restraints were obtained from ¹⁵N- or ¹³C-edited 3D NOESY spectra [Clore and Gronenborn, Meth. Enzymol. 239:249-363 (1994)]. φ-angle restraints were determined based on the ³J_{HN,H} coupling constants measured in a 3D HNHA spectrum [Clore and Gronenborn, Meth. Enzymol. 239:249-363 (1994)]. Slowly exchanging amide protons were identified from a series of 2D ¹⁵N—HSQC spectra recorded after the H₂O buffer was changed to a ²H₂O buffer. The intermolecular NOEs used in defining the structure of the bromodomain/Ac-histamine complex were detected in ¹³C-edited (F₁), ¹³C/¹⁵N-filtered (F₃) 3D NOESY spectrum [Clore and Gronenborn, Meth. Enzymol. 239:249-363 (1994)]. All NMR spectra were processed with the NMRPipe/NMRDraw programs and analyzed using NMR View [Johnson and Blevins, J. Biomol., NMR 4:603-614 (1994)].

Ligand titration experiments were performed by recording a series of 2D ¹⁵N—HSQC spectra on the uniformly ¹⁵N-labelled bromodomain (˜0.3 mM), respectively, in the presence of different amounts of ligand concentration ranging from 0 to ˜2.0 mM. The protein sample and the stock solutions of the ligands were all prepared in the same aqueous buffer containing 100 mM phosphate and 5 mM perdeuterated DTT at pH 6.5.

Structure calculations. Structures of the bromodomain were calculated with a distance geometry/simulated annealing protocol using the X-PLOR program [Brunger, X-PLOR Version 3.1: A system for X-Ray crystallography and NMR, Yale University Press, New Haven, Conn., (1993)]. A total of 1324 manually assigned NOE-derived distance restraints were obtained from the ¹⁵N- and ¹³C-edited NOE spectra. Further analysis of the NOE spectra was carried out by the iterative automated assignment procedure by using ARIA [Nilges and O'Donoghue, Prog. NMR Spectroscopy 32:107-139 (1998)], which integrates with X-PLOR for structure calculations. The ARIA-assigned distance restraints were in agreement with the structures calculated using only the manually assigned NOE distance restraints, hydrogen-bond distance restraints, and 54 φ-angle restraints. The final structure calculations employed a total of 2903 NMR experimental restraints obtained from the manual and the ARIA-assisted assignments. For the ensemble of the final 30 structures, no distance and torsional angle restraints were violated by more than 0.3 Å and 5 Å, respectively. The Lennard-Jones potential which was not used during any refinement stage, and stereochemistry of the final structures was validated with Ramachandran plot analysis by using Procheck-NMR [Laskowski et al., J. Biolmol. NMR 8:477-486 (1996)].

Site directed mutagenesis. Site directed mutagenesis was performed on selected residues of P/CAF Bromodomain using quick-change kit (Stratagene). The mutants were confirmed by sequencing and proteins were expressed and purified as above.

Peptide binding assay. Equal amount (10 μM) of GST, GST-P/CAF bromodomain and its mutant proteins, as well as various GST-fusion bromodomains from CBP and TIF1β were incubated for at least two hours at room temperature with the N-terminal biotinylated and lysine-acetylated Tat peptide (50 μM) in a 50 mM Tris buffer of pH 7.5, containing 50 mM NaCl, 0.1% BSA and 1 mM DTT. Streptavidin agarose (10 μL) was added to mixture and the beads were washed twice in the Tris buffer with 500 mM NaCl and 0.1% NP-40. Proteins were eluted from the argarose beads in SDS buffer and separated on a 14% SDS-PAGE. The resolved proteins were transferred onto nitrocellulose membrane (Pharmacia), and the membrane was blocked overnight with 5% non-fat milk in washing buffer of 20 mM Tris, pH 7.5, plus 150 mM NaCl and 0.1% Tween-20 at 4° C. Western blotting was performed with anti-GST antibody (Sigma) and goat anti-rabbit IgG conjugated with horseradish-peroxidase (Promega) and developed by chemiluminescence. Peptide competition experiments were performed by incubating various non-biotinylated and mutant Tat peptide with the P/CAF bromodomain and the biotinylated and wild type Tat peptide. The molar ratio of the wild type and mutant Tat peptides in the mixture were kept at 1:2. The binding results were analyses by using the procedure as described above. The full length protein sequence of the Human Immunodeficiency Virus type 1 Tat was obtained from GenBank, Accession No: AAA83395 (SEQ ID NO:45).

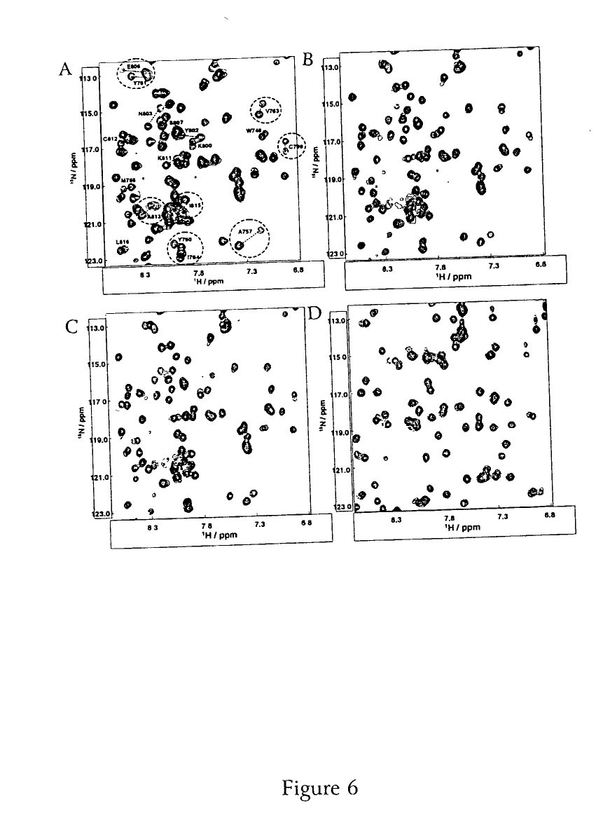

Results. To test whether or not the bromodomains of these HATs can bind to the lysine-acetylated Tat, in vitro binding assays were performed by using recombinant and purified bromodomains and lysine-acetylated peptides derived from the acetylation sites in Tat. While the bromodomains of CBP and TIF1β did not show any binding, the P/CAF bromodomain binds tightly only to the Tat peptide containing AcK50 (where AcK stands for an N^{ε}-acetyl lysine residue) (FIGS. 5A-5B). NMR binding studies further confirmed the specific interaction of the P/CAF bromodomain and lysine-acetylated Tat peptide. Because NMR resonances of amide protons are highly sensitive to local chemical environment and conformational change in a protein, two-dimensional ¹H-¹⁵N heteronuclear single quantum correlation (HSQC) spectrum can be used to detect even weak but specific interactions between a protein and its binding ligand. As shown in 2D HSQC spectra (FIGS. 6A-6D), the bromodomain of P/CAF binds weakly to the lysine-acetylated peptides derived from known acetylation sites of K28 on Tat and of K16 on histone H4 by only interacting with the acetyl-lysine residue in the peptides (K_{d}<300 μM). This is reflected the relatively small chemical shift perturbation of the amide proton signals of the protein upon addition of ligand. On the other hand, the P/CAF bromodomain interacts strongly with the Tat AcK50 peptide, which involves many protein residues in addition to those for acetyl-lysine binding with an estimated K_{d} of ˜20 μM. Binding of peptide residues flanking the acetyl-lysine may explain the high specificity of the P/CAF bromodomain for the acetylated Tat. Furthermore, the p300/CBP bromodomain did not bind the lysine-acetylated Tat peptide in a specific manner except its weak interaction with the acetyl-lysine residue in the peptide (FIGS. 6A-6D). Together, these results demonstrate the P/CAF bromodomain can specifically recognize the lysine-acetylated Tat involving K50.

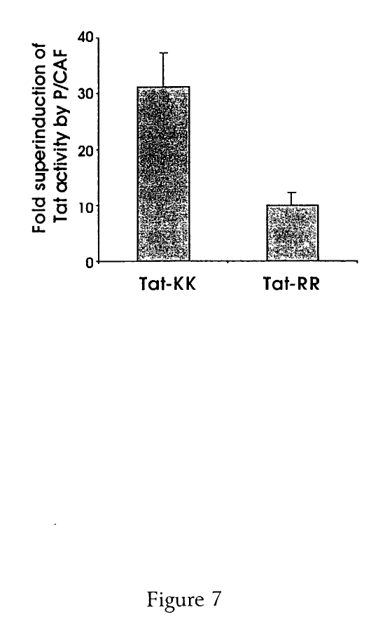

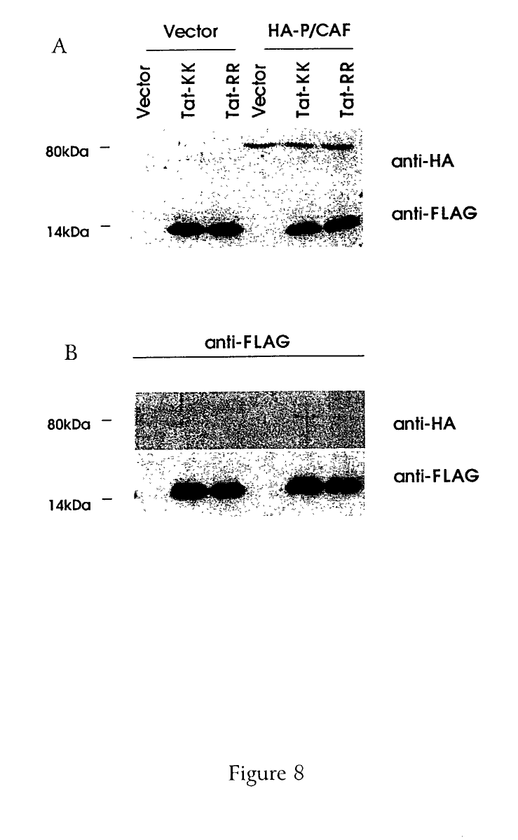

To determine how the P/CAF binding affects Tat function in vivo, transactivation activity of Tat was measured. Superinduction of Tat transactivation activity exhibited as much as a 30-fold increase upon P/CAF stimulation (FIG. 7). This profound P/CAF effect requires acetylation at K50 on Tat, as a double mutant of K50 and K51 substituted with arginines resulted in a nearly two-thirds reduction of the enhancement. Further, specific interaction between P/CAF and wild type Tat in cells was also detected, but not with the Tat double mutant containing K50R/K51R (FIGS. 8A-8B). Taken together, these results confirm that P/CAF can directly interact via its bromodomain with the lysine-acetylated Tat, which possibly regulates Tat transactivation activity.

To further understand the molecular basis of the P/CAF bromodomain recognition of the lysine-acetylated Tat, the three-dimensional structure was determined for the P/CAF bromodomain in complex with an 11-residue Tat peptide containing AcK50. A total of 2,903 NMR-derived distance and dihedral angle restraints were used. The structure of the bromodomain in the peptide-bound form consists of an up-and-down four-helix bundle (helices α_{Z}, α_{A}, α_{B}, and α_{C}) with a left-handed twist, and a long intervening loop between helices α_{Z} and α_{A} (termed the ZA loop) (FIG. 9). The overall structure of the complex is well defined (Table 9), and similar to the structure of the free bromodomain [Dhalluin et al., Nature 399:491-496 (1999); Example 1 above] except that the ZA and BC loops, which compose the acetyl-lysine binding pocket, undergo local conformational changes in order to accommodate their interactions with the peptide residues.

TABLE 9
NMR Structural Statistics of the P/CAF Bromodomain/Tat
Peptide Complex

| | |
|---|---|
| Total Experimental | 2903 |
| Restraints | |
| Distance Restraints^{a} | 2822 |
| Total Ambiguous | 122 |
| Total Unambiguous | 2700 |
| Intra-residue (i = j) | 1118 (41.40%) |
| Hydrogen Bond | 28 |
| Restraints | |
| Dihedral Angle | 53 |
| Restraints | |

Final Energies
(kcalmol⁻¹)

| E_{Total} | 366.35 ± 31.11 | |
|---|---|---|
| Ramachandran Plot (%) | Protein/Peptide Complex | Secondary Structure |
| Most Favorable Region | 72.06 ± 2.29 | 91.95 ± 3.04 |
| RMSDS of Atomic | | |
| Coordinates (Å) | Protein/Peptide Complex | Secondary Structure |
| Protein (aa 9-116) | | |
| Backbone | 0.66 ± 0.14 | 0.39 ± 0.05 |
| Heavy atoms | 1.25 ± 0.18 | 0.96 ± 0.07 |
| Peptide (aa 202-206, | | |
| 208-209) | | |
| Backbone | 0.50 ± 0.16 | |
| Heavy atoms | 1.83 ± 0.50 | |
| Complex (aa 9-116, | | |
| 202-206, 208-209) | | |
| Backbone | 0.72 ± 0.15 | 0.54 ± 0.09 |
| Heavy atoms | 1.39 ± 0.20 | 1.24 ± 0.16 |
| ^{a}Of the total 2903 NOE-derived distance restraints, only 341 were obtained by using ARIA program, of which 122 are classified as ambiguous NOEs. The latter resonance signals in the spectra match with more than one proton atom in both the chemical shift assignment and the final NMR structures. ^{b}The Lennard-Jones potential was not used during any refinement stage. ^{c}None of these final structures exhibit NOE-derived distance restraint violations greater than | | |
| # 0.5 Å or dihedral angle restraint violations greater than 5°. | | |

The Tat AcK50 peptide adopts an extended conformation and lies between the ZA and BC loops (FIG. 9). The acetyl-lysine side-chain intercalates deep into a preformed hydrophobic and aromatic cavity located between the ZA and BC loops opposite to the N- and C-termini, and interacts extensively with residues V752, Y760, 1764, Y802, and Y809. While the peptide residues S(AcK−4), K(AcK+1), R(AcK+2), R(AcK+5) do not interact directly with the protein, the residues Y(AcK−3), G(AcK−2), R(AcK−1), R(AcK+3), and Q(AcK+4) showed numerous intermolecular NOEs with the protein. Particularly, Y(AcK−3) and Q(AcK+4) form extensive contacts with V763 and E756, respectively, suggesting that these two residues contribute significantly to specificity of the bromodomain/Tat recognition.

To identify the amino acid residues of the P/CAF bromodomain that are important for complex formation, mutant proteins were tested for binding to the biotinylated and lysine-acetylated Tat peptide that is immobilized onto streptavidin agarose (FIG. 10A). As expected, proteins containing alanine point mutation at the residue Y809, Y802, V752, or F748, which interact directly with the acetyl-lysine residue, showed nearly complete loss or significantly reduced binding to the Tat peptide. Moreover, when the residue V763 or E756 was mutated to alanine, a nearly complete loss in binding to the Tat AcK50 peptide was observed, indicating that these two amino acid residues provide essential contributions to the Tat recognition by interacting with the residues flanking the acetyl-lysine. The results from the mutational analysis agree with the observations of intermolecular NOEs in the NMR spectra.

To further determine Tat sequence preference for P/CAF interaction, various mutant peptides were synthesized and their binding to the P/CAF bromodomain tested in a competition assay by using a western blot with the antibody against the GST-fusion bromodomain (FIG. 10B). Because of high sensitivity of this detection method, the binding assay was performed at protein concentration (˜10 μM) much lower than that in the NMR binding studies, which ensured specificity of protein-peptide interactions. In agreement with the binding results described above (e.g., see FIGS. 5A-5B, 6A-6D, 7, and 8A-8B), lysine-acetylated peptides derived from acetylation sites at K50 or K28 in Tat, or from histone H4 at K16 showed almost no competition with the Tat AcK50 peptide in binding to the P/CAF bromodomain, confirming that the latter interaction is tight and specific. Additionally, while substitution of residue R(AcK−1), K(AcK+1), R(AcK+2), or R(AcK+3) to alanine slightly weakened Tat peptide binding to the bromodomain, mutation of Y(AcK−3) or Q(AcK+4) resulted in significant loss in binding to the protein. These data can be explained by the observation of extensive pair-wise interactions between Y(AcK−3) and V763, and between Q(AcK+4) and E756, which agrees perfectly with the site-directed mutatagenesis results obtained with the protein (FIG. 10A). Together, these results demonstrate that the specificity of P/CAF bromodomain and acetylated Tat complex formation is achieved through specific interactions with acetyl-lysine as well as amino acid residues at (AcK−3) and (AcK+4) positions.

The HIV-1 Tat is a versatile protein and elicits many cellular functions. In addition to its lysine-acetylation and interaction with P/CAF as disclosed herein, this portion of arginine-rich motif (named ARM) has also been shown to interact with the TAR RNA element as well as protein nuclear localization, particularly involving arginine52 and arginine53. The findings disclosed herein that are based on the detailed structural and mutational analyses indicate that the lysine-acetylated Tat specifically is associated with P/CAF via a bromodomain interaction in vivo, and that this interaction is important for transactivation activity of Tat in cells. Further, the data disclosed herein reveal that in addition to the acetylated-lysine (K50) the flanking residues, tyrosine (AcK−3) and glutamine at (AcK+4) positions in Tat are also uniquely important for the specificity of the Tat and P/CAF bromodomain recognition, but not with its other functions. This new information is extremely useful in applying mutational analysis in in vivo studies to further elucidate the biological importance of the Tat-P/CAF association in molecular mechanisms by which Tat transactivates gene transcription of HIV-1 via chromatin remodeling.

What is claimed is:

1. A computer comprising a representation of a Tat-P/CAF complex in computer memory which comprises:
(a) a machine-readable data storage medium comprising a data storage material encoded with machine-readable data, wherein said data comprises structural coordinates from Tables 10-14;
(b) a working memory for storing instructions for processing said machine-readable data;
(c) a central processing unit coupled to said working memory and to said machine-readable data storage medium for processing said machine readable data into a three-dimensional representation of the Tat-P/CAF complex; and
(d) a display coupled to said central-processing unit for displaying said three-dimensional representation.

2. A method of identifying a compound that modulates the affinity of P/CAF for Tat that is acetylated at the lysine residue at position 50 of SEQ ID NO:45, said method comprising:
(a) contacting the bromodomain of P/CAF or a fragment thereof with a binding partner in the presence of the compound, wherein the bromodomain of P/CAF and the binding partner bind in the absence of the compound; and wherein the binding partner is selected from the group consisting of Tat that is acetylated at the lysine residue at position 50 of SEQ ID NO:45, a fragment of Tat comprising an acetyl-lysine at position 50, and an analog of the fragment of Tat comprising an acetyl-lysine at position 50; and
(b) measuring the affinity of the bromodomain of P/CAF and the binding partner; wherein a compound is identified as a compound that modulates the affinity of the bromodomain of P/CAF for Tat when there is a change in the affinity of the bromodomain of P/CAF for the binding partner in the presence of the compound.

3. The method of claim 2, wherein the affinity of the bromodomain of P/CAF for Tat increases in the presence of the compound; wherein the compound is identified as a Tat-P/CAF complex promoting agent.

4. The method of claim 2, wherein the affinity of the bromodomain of P/CAF for Tat decreases in the presence of the compound; wherein the compound is identified as an inhibitor of the Tat-P/CAF complex.

5. The method of claim 2, wherein the compound is selected by performing rational drug design with the set of atomic coordinates obtained from one or more of Tables 1-5 and 10-14, wherein said selecting is performed in conjunction with computer modeling.

6. A compound that is a small organic molecule identified by the method of claim 5, wherein the compound is an analog of acetyl-lysine, but with the proviso that the compound is not included in FIG. 13.

7. A method of identifying a compound that modulates the stability of the binding complex formed between P/CAF and Tat that is acetylated at the lysine residue at position 50 of SEQ ID NO: 45, the method comprising:
(a) contacting the bromodomain of P/CAF or a fragment thereof with a binding partner in the presence of the compound, wherein the bromodomain of P/CAF and the binding partner bind in the absence of the compound; and wherein the binding partner is selected from the group consisting of Tat that is acetylated at the lysine residue at position 50 of SEQ ID NO:45, a fragment of Tat comprising an acetyl-lysine at position 50, and an analog of the fragment of Tat comprising an acetyl-lysine at position 50; and
(b) measuring the stability of the binding complex between the bromodomain of P/CAF or a fragment thereof and the binding partner; wherein a compound is identified as a compound that modulates the stability of the Tat-P/CAF complex when there is a change in the stability of the binding complex between the bromodomain of P/CAF or a fragment thereof and the binding partner in the presence of the compound.

8. The method of claim 7, wherein the stability of the binding complex between the bromodomain of P/CAF or a fragment thereof and Tat or a fragment of Tat increases in the presence of the compound; wherein the compound is identified as a stabilizing agent.

9. The method of claim 7, wherein the stability of the binding complex between the bromodomain of P/CAF or a fragment thereof and Tat or a fragment of Tat decreases in the presence of the compound; wherein the compound is identified as an inhibitor.

10. The method of claim 7, wherein the compound is selected by performing rational drug design with the set of atomic coordinates obtained from one or more of Tables 1-5 and 10-14, wherein said selecting is performed in conjunction with computer modeling.

11. A compound that is a small organic molecule identified by the method of claim 10; wherein said compound is an analog of acetyl-lysine, but with the proviso that the compound is not included in FIG. 13.

12. An agent that can modulate the binding of P/CAF and Tat; wherein said agent is an analog of acetyl-lysine, but with the proviso that the agent is not included in FIG. 13.

13. The agent of claim 12 that inhibits and/or destabilizes the binding of P/CAF and Tat.

14. A method of preventing, retarding the progression and/or treating HIV infection in an individual comprising administering to the individual a compound that inhibits the binding of P/CAF and Tat and/or destabilizes the Tat-P/CAF complex.

15. The method of claim 14, wherein the compound is an acetyl-lysine analog.

16. The method of claim 15, wherein said acetyl-lysine analog is contained in FIG. 13.

17. A method of preventing, retarding the progression and/or treating HIV infection in an individual comprising administering an acetyl-lysine analog to the individual; wherein said acetyl-lysine analog was identified by the method of claim 10 as a compound that modulates the stability of the binding complex formed between P/CAF and Tat.

18. The method of claim 17, wherein said acetyl-lysine analog is contained in FIG. 13.

19. A method of preventing, retarding the progression and/or treating HIV infection in an individual comprising administering an acetyl-lysine analog to the individual; wherein said acetyl-lysine analog was identified by the method of claim 5 as a compound that modulates the affinity of P/CAF for Tat.

20. The method of claim 19 wherein said acetyl-lysine analog is contained in FIG. 13.


SEQUENCE LISTING (60 sequences)

SEQ ID NO: 1 - DNA, 3014 bp, Homo sapiens
ggggccgcgtcgacgcggaaaagaggccgtggggggcctcccagcgctggcagacaccgtgaggctggcagccgccggcacgcacacctagtccgcagtcccgaggaacatgtccgcagccagggcgcggagcagagtcccgggcaggagaaccaagggagggcgtgtgctgtggcggcggcggcagcggcagcggagccgctagtcccctccctcctgggggagcagctgccgccgctgccgccgccgccaccaccatcagcgcgcggggcccggccagagcgagccgggcgagcggcgcgctagggggagggcgggggcggggaggggggtgggcgaagggggcgggagggcgtggggggagggtctcgctctcccgactaccagagcccgagggagaccctggcggcggcggcggcgcctgacactcggcgcctcctgccgtgctccggggcggcatgtccgaggctggcggggccgggccgggcggctgcggggcaggagccggggcaggggccgggcccggggcgctgcccccgcagcctgcggcgcttccgcccgcgcccccgcagggctccccctgcgccgctgccgccgggggctcgggcgcctgcggtccggcgacggcagtggctgcagcgggcacggccgaaggaccgggaggcggtggctcggcccgaatcgccgtgaagaaagcgcaactacgctccgctccgcgggccaagaaactggagaaactcggagtgtactccgcctgcaaggccgaggagtcttgtaaatgtaatggctggaaaaaccctaacccctcacccactccccccagagccgacctgcagcaaataattgtcagtctaacagaatcctgtcggagttgtagccatgccctagctgctcatgtttcccacctggagaatgtgtcagaggaagaaatgaacagactcctgggaatagtattggatgtggaatatctctttacctgtgtccacaaggaagaagatgcagataccaaacaagtttatttctatctatttaagctcttgagaaagtctattttacaaagaggaaaacctgtggttgaaggctctttggaaaagaaacccccatttgaaaaacctagcattgaacagggtgtgaataactttgtgcagtacaaatttagtcacctgccagcaaaagaaaggcaaacaatagttgagttggcaaaaatgttcctaaaccgcatcaactattggcatctggaggcaccatctcaacgaagactgcgatctcccaatgatgatatttctggatacaaagagaactacacaaggtggctgtgttactgcaacgtgccacagttctgcgacagtctacctcggtacgaaaccacacaggtgtttgggagaacattgcttcgctcggtcttcactgttatgaggcgacaactcctggaacaagcaagacaggaaaaagataaactgcctcttgaaaaacgaactctaatcctcactcatttcccaaaatttctgtccatgctagaagaagaagtatatagtcaaaactctcccatctgggatcaggattttctctcagcctcttccagaaccagccagctaggcatccaaacagttatcaatccacctcctgtggctgggacaatttcatacaattcaacctcatcttcccttgagcagccaaacgcagggagcagcagtcctgcctgcaaagcctcttctggacttgaggcaaacccaggagaaaagaggaaaatgactgattctcatgttctggaggaggccaagaaaccccgagttatgggggatattccgatggaattaatcaacgaggttatgtctaccatcacggaccctgcagcaatgcttggaccagagaccaattttctgtcagcacactcggccagggatgaggcggcaaggttggaagagcgcaggggtgtaattgaatttcacgtggttggcaattccctcaaccagaaaccaaacaagaagatcctgatgtggctggttggcctacagaacgttttctcccaccagctgccccgaatgccaaaagaatacatcacacggctcgtctttgacccgaaacacaaaacccttgctttaattaaagatggccgtgttattggtggtatctgtttccgtatgttcccatctcaaggattcacagagattgtcttctgtgctgtaacctcaaatgagcaagtcaagggctatggaacacacctgatgaatcatttgaaagaatatcacataaagcatgacatcctgaacttcctcacatatgcagatgaatatgcaattggatactttaagaaacagggtttctccaaagaaattaaaatacctaaaaccaaatatgttggctatatcaaggattatgaaggagccactttaatgggatgtgagctaaatccacggatcccgtacacagaattttctgtcatcattaaaaagcagaaggagataattaaaaaactgattgaaagaaaacaggcacaaattcgaaaagtttaccctggactttcatgttttaaagatggagttcgacagattcctatagaaagcattcctggaattagagagacaggctggaaaccgagtggaaaagagaaaagtaaagagcccagagaccctgaccagctttacagcacgctcaagagcatcctccagcaggtgaagagccatcaaagcgcttggcccttcatggaacctgtgaagagaacagaagctccaggatattatgaagttataaggttccccatggatctgaaaaccatgagtgaacgcctcaagaataggtactacgtgtctaagaaattattcatggcagacttacagcgagtctttaccaattgcaaagagtacaacgccgctgagagtgaatactacaaatgtgccaatatcctggagaaattcttcttcagtaaaattaaggaagctggattaattgacaagtgattttttttccccctctgcttcttagaaactcaccaagcagtgtgcctaaagcaaggt

SEQ ID NO: 2 - protein, 832 aa, Homo sapiens
MSEAGGAGPGGCGAGAGAGAGPGALPPQPAALPPAPPQGSPCAAAAGGSGACGPATAVAAAGTAEGPGGGGSARIAVKKAQLRSAPRAKKLEKLGVYSACKAEESCKCNGWKNPNPSPTPPRADLQQIIVSLTESCRSCSHALAAHVSHLENVSEEEMNRLLGIVLDVEYLFTCVHKEEDADTKQVYFYLFKLLRKSILQRGKPVVEGSLEKKPPFEKPSIEQGVNNFVQYKFSHLPAKERQTIVELAKMFLNRINYWHLEAPSQRRLRSPNDDISGYKENYTRWLCYCNVPQFCDSLPRYETTQVFGRTLLRSVFTVMRRQLLEQARQEKDKLPLEKRTLILTHFPKFLSMLEEEVYSQNSPIWDQDFLSASSRTSQLGIQTVINPPPVAGTISYNSTSSSLEQPNAGSSSPACKASSGLEANPGEKRKMTDSHVLEEAKKPRVMGDIPMELINEVMSTITDPAAMLGPETNFLSAHSARDEAARLEERRGVIEFHVVGNSLNQKPNKKILMWLVGLQNVFSHQLPRMPKEYITRLVFDPKHKTLALIKDGRVIGGICFRMFPSQGFTEIVFCAVTSNEQVKGYGTHLMNHLKEYHIKHDILNFLTYADEYAIGYFKKQGFSKEIKIPKTKYVGYIKDYEGATLMGCELNPRIPYTEFSVIIKKQKEIIKKLIERKQAQIRKVYPGLSCFKDGVRQIPIESIPGIRETGWKPSGKEKSKEPRDPDQLYSTLKSILQQVKSHQSAWPFMEPVKRTEAPGYYEVIRFPMDLKTMSERLKNRYYVSKKLFMADLQRVFTNCKEYNAAESEYYKCANILEKFFFSKIKEAGLIDK

SEQ ID NO: 3 - protein, 16 aa, Artificial Sequence
  synthetic bromodomain peptide
  Xaa (2)..(4): Xaa is a maximum of three amino acids. Each of these can be any amino acid. One may be missing.
  Xaa (4)..(11): Xaa is a maximum of eight amino acids. Each of these can be any amino acid. One, two, or three may be missing.
  Xaa (5)..(5): Xaa is a single amino acid that is either Pro, Lys, or His.
  Xaa (6)..(6): Xaa is any single amino acid.
  Xaa (8)..(8): Xaa is a single amino acid that can be either Tyr, Phe, or His.
  Xaa (9)..(13): Xaa is any amino acid.
  Xaa (15)..(15): Xaa is a single amino acid that can be either Met, Ile, or Val.
FXPXXXYXXXXXXPXD

SEQ ID NO: 4 - protein, 12 aa, Artificial Sequence
  synthetic bromodomain peptide
  Xaa (6)..(6): Xaa represents an acetyl-lysine
ISYGRXKRRQRR

SEQ ID NO: 5 - protein, 14 aa, Artificial Sequence
  synthetic bromodomain peptide
  Xaa (8)..(8): Xaa represents an acetyl lysine.
ARKSTGGXAPRKQL

SEQ ID NO: 6 - protein, 14 aa, Artificial Sequence
  synthetic bromodomain peptide
  Xaa (8)..(8): Xaa represents an acetyl lysine.
QSTSRHKXLMFKTE

SEQ ID NO: 7 - protein, 110 aa, Homo sapiens, bromodomain peptide
SKEPRDPDQLYSTLKSILQQVKSHQSAWPFMEPVKRTEAPGYYEVIRSPMDLKTMSERLKNRYYVSKKLFMADLQRVFTNCKEYNAPESEYYKCANILEKFFFSKIKEAG

SEQ ID NO: 8 - protein, 110 aa, Homo sapiens
GKELKDPDQLYTTLKNLLAQIKSHPSAWPFMEPVKKSEAPDYYEVIRFPIDLKTMTERLRSRYYVTRKLFVADLQRVIANCREYNPPDSEYCRCASALEKFFYFKLKEGG

SEQ ID NO: 9 - protein, 109 aa, Tetrahymena thermophila
LKKSKERSFNLQCANVIENMKRHKQSWPFLDPVNKDDVPDYYDVITDPIDIKAIEKKLQNNQYVDKDQFIKDVKRIFTNAKIYNQPDTIYYKAAKELEDFVEPYLTKLK

SEQ ID NO: 10 - protein, 109 aa, Saccharomyces cerevisiae
AQRPKRGPHDAAIQNILTELQNHAAAWPFLQPVNKEEVPDYYDFIKEPMDLSTMEIKLESNKYQKMEDFIYDARLVFNNCRMYNGENTSYYKYANRLEKFFNNKVKEIP

SEQ ID NO: 11 - protein, 112 aa, Homo sapiens
KKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVM

SEQ ID NO: 12 - protein, 112 aa, Homo sapiens
KKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVM

SEQ ID NO: 13 - protein, 112 aa, Mus musculus
KKIFKPEELRQALMPTLEALYRQDPESLPFRQPVDPQLLGIPDYFDIVKNPMDLSTIKRKLDTGQYQEPWQYVDDVRLMFNNAWLYNRKTSRVYKFCSKLAEVFEQEIDPVM

SEQ ID NO: 14 - protein, 111 aa, Caenorhabditis elegans
DTVFSQEDLIKFLLPVWEKLDKSEDAAPFRVPVDAKLLNIPDYHEIIKRPMDLETVHKKLYAGQYQNAGQFCDDIWLMLDNAWLYNRKNSKVYKYGLKLSEMFVSEMDPVM

SEQ ID NO: 15 - protein, 110 aa, Homo sapiens
RRRTDPMVTLSSILESIINDMRDLPNTYPFHTPVNAKVVKDYYKIITRPMDLQTLRENVRKRLYPSREEFREHLELIVKNSATYNGPKHSLTQISQSMLDLCDEKLKEKE

SEQ ID NO: 16 - protein, 110 aa, Mesocricetus auratus; identical to SEQ ID NO: 15

SEQ ID NO: 17 - protein, 111 aa, Homo sapiens
LLDDDDQVAFSFILDNIVTQKMMAVPDSWPFHHPVNKKFVPDYYKVIVNPMDLETIRKNISKHKYQSRESFLDDVNLILANSVKYNGPESQYTKTAQEIVNVCYQTLTEYD

SEQ ID NO: 18 - protein, 111 aa, Mesocricetus auratus
LLDDDDQVAFSFILDNIVTQKMMAVPDSWPFHHPVNKKFVPDYYKVIVSPMDLETIRKNISKHKYQSRESFLDDVNLILANSVKYNGSESQYTKTAQEIVNVCYQTLTEYD

SEQ ID NO: 19 - protein, 111 aa, Homo sapiens
KPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMP

SEQ ID NO: 20 - protein, 111 aa, Homo sapiens
KPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMP

SEQ ID NO: 21 - protein, 111 aa, Drosophila melanogaster
RPGRNTNQLQYLIKTVMKVIWKHHFSWPFQQPVDAKKLNLPDYHKIIKQPMDMGTIKKRLENNYYWSAKETIQDFNTMFNNCYVYNKPGEDVVVMAQTLEKVFLQKIESMP

SEQ ID NO: 22 - protein, 109 aa, Saccharomyces cerevisiae
NPIPKHQQKHALLAIKAVKRLKDARPFLQPVDPVKLDIPFYFNYIKRPMDLSTIERKLNVGAYEVPEQITEDFNLMVNNSIKFNGPNAGISQMARNIQASFEKHMLNMP

SEQ ID NO: 23 - protein, 113 aa, Homo sapiens
KKGKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMP

SEQ ID NO: 24 - protein, 113 aa, Homo sapiens
KKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMP

SEQ ID NO: 25 - protein, 113 aa, Drosophila melanogaster
NKEKLSDALKSCNEILKELFSKKHSGYAWPFYKPVDAEMLGLHDYHDIIKKPMDLGTVKRKMDNREYKSAPEFAADVRLIFTNCYKYNPPDHDVVAMGRKLQDVFEMRYANIP

SEQ ID NO: 26 - protein, 113 aa, Saccharomyces cerevisiae
KSKRLQQAMKFCQSVLKELMAKKHASYNYPFLEPVDPVSMNLPTYFDYVKEPMDLGTIAKKLNDWQYQTMEDFEREVRLVFKNCYTFNPDGTIVNMMGHRLEEVFNSKWADRP

SEQ ID NO: 27 - protein, 108 aa, Homo sapiens
MEMQLTPFLILLRKTLEQLQEKDTGNIFSEPVPLSEVPDYLDHIKKPMDFFTMKQNLEAYRYLNFDDFEEDFNLIVSNCLKYNAKDTIFYRAAVRLREQGGAVVRQAR

SEQ ID NO: 28 - protein, 113 aa, Homo sapiens
SEDQEAIQAQKIWKKAIMLVWRAAANHRYANVFLQPVTDDIAPGYHSIVQRPMDLSTIKKNIENGLIRSTAEFQRDIMLMFQNAVMYNSSDHDVYHMAVEMQRDVLEQIQQFL

SEQ ID NO: 29 - protein, 106 aa, Gallus gallus
NLPTVDPIAVCHELYNTIRDYKDEQGRLLCELFIRAPKRRNQPDYYEVVSQPIDLMKIQQKLKMEEYDDVNVLTADFQLLFNNAKAYYKPDSPEYKAACKLWELYL

SEQ ID NO: 30 - protein, 112 aa, Gallus gallus
SSPGYLKEILEQLLEAVAVATNPSGRLISELFQKLPSKVQYPDYYAIIKEPIDLKTIAQRIQNGTYKSIHAMAKDIDLLAKNAKTYNEPGSQVFKDANAIKKIFNMKKAEIE

SEQ ID NO: 31 - protein, 112 aa, Gallus gallus
TSFMDTSNPLYQLYDTVRSCRNNQGQLISEPFFQLPSKKKYPDYYQQIKTPISLQQIRAKLKNHEYETLDQLEADLNLMFENAKRYNVPNSAIYKRVLKMQQVMQAKKKELA

SEQ ID NO: 32 - protein, 113 aa, Gallus gallus
SKKNMRKQRMKILYNAVLEARESGTQRRLCDLFMVKPSKKDYPDYYKIILEPMDLKMIEHNIRNDKYVGEEAMIDDMKLMFRNARHYNEEGSQVYNDAHMLEKILKEKRKELG

SEQ ID NO: 33 - protein, 115 aa, Gallus gallus
KKSKYMTPMQQKLNEVYEAVKNYTDKRGRRLSAIFLRLPSRSELPDYYITIKKPVDMEKIRSHMMANKYQDIDSMVEDFVMMFNNACTYNEPESLIYKDALVLHKVLLETRREIE

SEQ ID NO: 34 - protein, 112 aa, Description of unknown organism, see Jeanmougin et al., Trends in Biochem. Sci. 22:151-153 (1997)
HNAPFDKTKFDEVLEALVGLKDNEGNPFDDIFEELPSKRYFPDYYQIIQKPICYKMMRNKAKTGKYLSMGDFYDDIRLMVSNAQTYNMPGSLVYECSVLIANTANSLESKDG

SEQ ID NO: 35 - protein, 113 aa, Description of unknown organism, see Jeanmougin et al., Trends in Biochem. Sci. 22:151-153 (1997)
GTNEIDVPKVIQNILDALHEEKDEQGRFLIDIFIDLPSKRLYPDYYEIIKSPMTIKMLEKRFKKGEYTTLESFVKDLNQMFINAKTYNAPGSFVYEDAEKLSQLSSSLISSFS

SEQ ID NO: 36 - protein, 113 aa, Homo sapiens; identical to SEQ ID NO: 35

SEQ ID NO: 37 - protein, 114 aa, Homo sapiens
SPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIE

SEQ ID NO: 38 - protein, 113 aa, Gallus gallus
SPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIA

SEQ ID NO: 39 - protein, 114 aa, Gallus gallus
SPNPPNLTKKMKKIVDAVIKYKDSSSGRQLSEVFIQLPSRKELPEYYELIRKPVDFKKIKERIRNHKYRSLNDLEKDVMLLCQNAQTFNLEVSLIYEDSIVLQSVFTSVRQKIE

SEQ ID NO: 40 - protein, 105 aa, Homo sapiens
AKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNE

SEQ ID NO: 41 - protein, 105 aa, Mus musculus
AKLSPANQRKCERVLLALFCHEPCRPLHQLATDSTFSMEQPGGTLDLTLIRARLQEKLSPPYSSPQEFAQDVGRMFKQFNKLTEDKADVQSIIGLQRFFETRMND

SEQ ID NO: 42 - protein, 108 aa, Mus sp.
TKLTPIDKRKCERLLLFLYCHEMSLAFQDPVPLTVPDYYKIIKNPMDLSTIKKRLQEDYCMYTKPEDFVADFRLIFQNCAEFNEPDSEVANAGIKLESYFEELLKNLY

SEQ ID NO: 43 - protein, 18 aa, Artificial Sequence
  synthetic bromodomain peptide
  Xaa (1)..(1): Xaa can be any single amino acid
  Xaa (2)..(2): Xaa can be any single amino acid
  Xaa (4)..(6): Xaa is a maximum of three amino acids. Each of these can be any amino acid. One may be missing.
  Xaa (6)..(13): Xaa is a maximum of eight amino acids. Each of these can be any amino acid. One, two, or three may be missing.
  Xaa (7)..(7): Xaa is a single amino acid that can be Pro, Lys, or His.
  Xaa (8)..(8): Xaa is a single amino acid that can be any amino acid.
  Xaa (10)..(10): Xaa is a single amino acid that can be a Tyr, Phe, or His.
  Xaa (11)..(15): Xaa is any amino acid.
  Xaa (17)..(17): Xaa is a single amino acid that can be Met, Ile, or Val.
XXFXPXXXYXXXXXXPXD

SEQ ID NO: 44 - protein, 20 aa, Artificial Sequence
  synthetic bromodomain peptide
WPFMEPVKRTEAPGYYEVIR

SEQ ID NO: 45 - protein, 101 aa, Human immunodeficiency virus type 1 Tat protein
MEPVDPRLEPWKHPGSQPKTASNNCYCKRCCLHCQVCFTKKGLGISYGRKKRRQRRRAPQDSKTHQVSLSKQPASQPRGDPTGPKESKKKVERETETDPED

SEQ ID NO: 46 - protein, 7 aa, Artificial Sequence
  Synthetic HIV-1 Tat peptide
  Xaa (5)..(7): Xaa is a maximum of three amino acids. Each amino acid can be any amino acid. One or two may be missing.
YGRKXRQ

SEQ ID NO: 47 - protein, 10 aa, Artificial Sequence
  synthetic HIV-1 Tat peptide
SYGRKKRRQR

SEQ ID NO: 48 - protein, 10 aa, Artificial Sequence
  synthetic bromodomain peptide
  Xaa (2)..(5): Xaa is a maximum of four amino acids. Each of these can be any amino acid. One or two may be missing.
  Xaa (4)..(7): Xaa is a maximum of four amino acids. Each of these can be any amino acid. One or two may be missing.
  Xaa (6)..(9): Xaa is a maximum of four amino acids. Each of these can be any amino acid. One or two may be missing.
  Xaa (8)..(10): Xaa is a maximum of three amino acids. Each of these can be any amino acid. One or two may be missing.
  Xaa (10)..(10): Xaa is a single amino acid that is either Ile, Leu, Met, or Val.
FXVXEXYXVX

SEQ ID NO: 49 - protein, 62 aa, Artificial Sequence
  synthetic bromodomain peptide
FMEPVKRTEAPGYYEVIRFPMDLKTMSERLKNRYYVSKKLFMADLQRVFTNCKEYNAAESEY

SEQ ID NO: 50 - protein, 11 aa, Artificial Sequence
  synthetic HIV-1 Tat peptide
  Xaa (5)..(5): Xaa is an acetylated lysine (AcK).
SYGRXKRRQRC

SEQ ID NO: 51 - protein, 11 aa, Artificial Sequence
  synthetic HIV-1 Tat peptide
  Xaa (5)..(5): Xaa is an acetylated lysine (AcK).
SAGRXKRRQRC

SEQ ID NO: 52 - protein, 11 aa, Artificial Sequence
  synthetic HIV-1 Tat peptide
  Xaa (5)..(5): Xaa ia an acetylated lysine (AcK).
SYGAXKRRQRC

SEQ ID NO: 53 - protein, 11 aa, Artificial Sequence
  synthetic HIV-1 Tat peptide
  Xaa (5)..(5): Xaa is an acetylated lysine (AcK).
SYGRXARRQRC

SEQ ID NO: 54 - protein, 11 aa, Artificial Sequence
  synthetic HIV-1 Tat peptide
  Xaa (5)..(5): Xaa is an acetylated lysine (AcK).
SYGRXKARQRC

SEQ ID NO: 55 - protein, 11 aa, Artificial Sequence
  synthetic HIV-1 Tat peptide
  Xaa (5)..(5): Xaa is an acetylated lysine (AcK)
SYGRXKRAQRC

SEQ ID NO: 56 - protein, 11 aa, Artificial Sequence
  synthetic HIV-1 Tat peptide
  Xaa (5)..(5): Xaa is an acetylated lysine (AcK)
SYGRXKRRARC

SEQ ID NO: 57 - protein, 11 aa, Artificial Sequence
  synthetic HIV-1 Tat peptide
  Xaa (6)..(6): Xaa is an acetylated lysine (AcK)
SYGRKXRRQRC

SEQ ID NO: 58 - protein, 11 aa, Artificial Sequence
  synthetic HIV-1 Tat peptide
  Xaa (7)..(7): Xaa is an acetylated lysine (AcK)
TNCYCKXCCFH

SEQ ID NO: 59 - protein, 20 aa, Artificial Sequence
  synthetic histone H4 AcK16 peptide
  Xaa (16)..(16): Xaa is an acetylated lysine (AcK)
SGRGKGGKGLGKGGAXRHRK

SEQ ID NO: 60 - protein, 11 aa, Artificial Sequence
  synthetic HIV-1 Tat peptide
SYGRKKRRQRC